(19) United States
(12) Patent Application Publication
(10) Pub. No.: US 2008/0280774 A1
(43) Pub. Date: Nov. 13, 2008

(54) Methods and Systems for Diagnosis, Prognosis and Selection of Treatment of Leukemia

(75) Inventors: Michael Edward Burczynski, Collegeville, PA (US); Jennifer A. Stover, Topsfield, MA (US); Frederick William Immermann, Suffern, NY (US); Andrew J. Dorner, Lexington, MA (US); Natalie C. Twine, Goffstown, NH (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/884,169

(22) PCT Filed: Feb. 16, 2006
(86) PCT No.: PCT/US06/05855

§ 371 Date: Apr. 24, 2008

Related U.S. Application Data
(60) Provisional application No. 60/653,117, filed on Feb. 16, 2005.

(51) Int. Cl. C40B 30/04; C12Q 1/68; C40B 40/08; G01N 33/50

(52) U.S. Cl. 506/9; 435/6; 506/17; 702/20

(57) ABSTRACT

The present invention provides methods, systems and equipment for the prognosis, diagnosis and selection of treatment of AML or other types of leukemia. Genes prognostic of clinical outcome of leukemia patients can be identified according to the present invention. Leukemia disease genes can also be identified according to the present invention. These genes are differentially expressed in PBMCs of AML patients relative to disease-free humans. These genes can be used for the diagnosis or monitoring the development, progression or treatment of AML.

### CROSS-REFERENCE TO RELATED APPLICATIONS

This application claims the benefit of U.S. Ser. No. 60/653,117, filed Feb. 16, 2005.

### TECHNICAL FIELD

The present invention relates to leukemia diagnostic and prognostic genes and methods of using the same for the diagnosis, prognosis, and selection of treatment of AML or other types of leukemia.

### BACKGROUND

Acute myeloid leukemia (AML) is a heterogeneous clonal disorder typified by hyperproliferation of immature leukemic blast cells in the bone marrow. Approximately 90% of all AML cases exhibit proliferation of CD33⁺ blast cells, and CD33 is a cell surface antigen that appears to be specifically expressed in myeloblasts and myeloid progenitors but is absent from normal hematopoetic stem cells. Gemtuzumab ozogamicin (Mylotarg® or GO) is an anti-CD33 antibody conjugated to calicheamicin specifically designed to target CD33⁺ blast cells of AML patients for destruction. For reviews, see Matthews, LEUKEMIA, 12(Suppl 1):S33-S36 (1998); and Bernstein, LEUKEMIA, 14:474-475 (2000).

While gemtuzumab ozogamicin has demonstrated efficacy in patients with advanced AML, it is sometimes not completely effective as a single line agent. Both in vitro and in vivo studies have demonstrated that p-glycoprotein expression and the multi-drug resistance (MDR) phenotype are associated with reduced responsiveness to gemtuzumab ozogamicin therapy, suggesting that extrusion of gemtuzumab ozogamicin by this mechanism may be one of several important molecular pathways of gemtuzumab ozogamicin resistance (Naito, et al., LEUKEMIA, 14:1436-1443 (2000); and Linenberger, et al., BLOOD, 98:988-994 (2001)). However, the MDR phenotype fails to account for all cases found to be gemtuzumab ozogamicin resistant. While gemtuzumab ozogamicin exhibits a favorable safety profile in the majority of patients receiving Mylotarg® therapy (Sievers, et al., J CLIN. ONCOL., 19(13):3244-3254 (2001)), a small but significant number of cases of hepatic veno-occlusive disease have been reported following exposure to this therapy (Neumeister, et al., ANN. HEMATOL., 80:119-120 (2001)). Recently, GO has also been evaluated in combination with an anthracycline and cytarabine in an attempt to increase the effectiveness of GO administered as a single agent therapy (Alvarado, et al., CANCER CHEMOTHER PHARMACOL., 51:87-90 (2003)).

### SUMMARY OF THE INVENTION

It is therefore an object of the present invention to provide effective pharmacogenomic analysis to assess any relationship between gene expression and response to therapy.

It is an object of the present invention to identify leukemia prognostic genes whose expression levels are predictive of clinical outcome of leukemia patients who undergo an anti-cancer therapy.

It is a further object of the present invention to provide a method for predicting a clinical outcome of a leukemia patient as well as a method for selecting a treatment for a leukemia patient based on pharmacogenomic analysis.

It is another object of the present invention to identify leukemia diagnostic genes and to provide a method for diagnosis, or monitoring the occurrence, development, progression or treatment, of a leukemia based on the analysis of the expression levels of the diagnostic genes.

Thus, in one aspect, the present invention provides a method for predicting a clinical outcome in response to a treatment of a leukemia. The method includes the following steps: (1) measuring expression levels of one or more prognostic genes of the leukemia in a peripheral blood mononuclear cell sample derived from a patient prior to the treatment; and (2) comparing each of the expression levels to a corresponding control level, wherein the result of the comparison is predictive of a clinical outcome. “Prognostic genes” referred to in the application include, but are not limited to, any genes that are differentially expressed in peripheral blood mononuclear cells (PBMCs) or other tissues of leukemia patients with different clinical outcomes. In particular, prognostic genes include genes whose expression levels in PBMCs or other tissues of leukemia patients are correlated with clinical outcomes of the patients. Exemplary prognostic genes are shown in Table 1, Table 2, Table 3, Table 4, Table 5 and Table 6. A “clinical outcome” referred to in the application includes, but is not limited to, any response to any leukemia treatment.

The present invention is suitable for prognosis of any leukemias, including acute leukemia, chronic leukemia, lymphocytic leukemia or nonlymphocytic leukemia. In particular, the present invention is suitable for prognosis of acute myeloid leukemia (AML). Typically, the clinical outcome is measured by a response to an anti-cancer therapy. For example, the anti-cancer therapy includes administering one or more compounds selected from the group consisting of an anti-CD33 antibody, a daunorubicin, a cytarabine, a gemtuzumab ozogamicin, an anthracycline, and a pyrimidine or purine nucleotide analog. In one particular example, the present invention may be used to predict a response to a gemtuzumab ozogamicin (GO) combination therapy.

In one embodiment, the one or more prognostic genes suitable for the invention include at least a first gene selected from a first class and a second gene selected from a second class. The first class includes genes having higher expression levels in peripheral blood mononuclear cells in patients predicted to have a less desirable clinical outcome in response to the treatment. Exemplary first class genes are shown in Table 1 and Table 3. The second class includes genes having higher expression levels in peripheral blood mononuclear cells in patients predicted to have a more desirable clinical outcome in response to the treatment. Exemplary second class genes are shown in Table 2 and 4. In one embodiment, the first gene is selected from Table 3 and the second gene is selected from Table 4.

In one particular embodiment, the first gene is selected from the group consisting of zinc finger protein 217, peptide transporter 3, forkhead box O3A, T cell receptor alpha locus and putative chemokine receptor/GTP-binding protein, and the second gene is selected from the group consisting of metallothionein, fatty acid desaturase 1, an uncharacterized gene corresponding to Affymetrix ID 216336, deformed epidermal autoregulatory factor 1 and growth arrest and DNA-damage-inducible alpha. In another embodiment, the first gene is serum glucocorticoid regulated kinase and the second gene is metallothionein 1X/1L.

In some embodiments, each of the expression levels of the prognostic genes is compared to the corresponding control level which is a numerical threshold.

In some embodiments, the method of the present invention may be used to predict development of an adverse event in a leukemia patient in response to a treatment. For example, the method may be used to assess the possibility of development of veno-occlusive disease (VOD). Exemplary prognostic genes predictive of VOD are shown in Table 5 and Table 6. In one particular embodiment, the expression level of p-selectin ligand is measured to predict the risk for VOD.

In another aspect, the present invention provides a method for predicting a clinical outcome of a leukemia by taking the following steps: (1) generating a gene expression profile from a peripheral blood sample of a patient having the leukemia; and (2) comparing the gene expression profile to one or more reference expression profiles, wherein the gene expression profile and the one or more reference expression profiles contain expression patterns of one or more prognostic genes of the leukemia in peripheral blood mononuclear cells, and wherein the difference or similarity between the gene expression profile and the one or more reference expression profiles is indicative of the clinical outcome for the patient.

In one embodiment, the gene expression profile of the one or more prognostic genes may be compared to the one or more reference expression profiles by, for example, a k-nearest neighbor analysis or a weighted voting algorithm. Typically, the one or more reference expression profiles represent known or determinable clinical outcomes. In some embodiments, the gene expression profile from the patient may be compared to at least two reference expression profiles, each of which represents a different clinical outcome. For example, each reference expression profile may represent a different clinical outcome selected from the group consisting of remission to less than 5% blasts in response to the anti-cancer therapy; remission to no less than 5% blasts in response to the anti-cancer therapy; and non-remission in response to the anti-cancer therapy. In some embodiments, the one or more reference expression profiles may include a reference expression profile representing a leukemia-free human.

In some embodiments, the gene expression profile may be generated by using a nucleic acid array. Typically, the gene expression profile is generated from the peripheral blood sample of the patient prior to the anti-cancer therapy.

In one embodiment, the one or more prognostic genes include one or more genes selected from Table 3 or Table 4. In another embodiment, the one or more prognostic genes include ten or more genes selected from Table 3 or Table 4. In yet another embodiment, the one or more prognostic genes include twenty or more genes selected from Table 3 or Table 4.

In yet another aspect, the present invention provides a method for selecting a treatment for a leukemia patient. The method includes the following steps: (1) generating a gene expression profile from a peripheral blood sample derived from the leukemia patient; (2) comparing the gene expression profile to a plurality of reference expression profiles, each representing a clinical outcome in response to one of a plurality of treatments; and (3) selecting from the plurality of treatments a treatment which has a favorable clinical outcome for the leukemia patient based on the comparison in step (2), wherein the gene expression profile and the one or more reference expression profiles comprise expression patterns of one or more prognostic genes of the leukemia in peripheral blood mononuclear cells. In one embodiment, the gene expression profile may be compared to the plurality of reference expression profiles by, for example, a k-nearest neighbor analysis or a weighted voting algorithm.

In one embodiment, the one or more prognostic genes include one or more genes selected from Table 3 or Table 4. In another embodiment, the one or more prognostic genes include ten or more genes selected from Table 3 or Table 4. In yet another embodiment, the one or more prognostic genes include twenty or more genes selected from Table 3 or Table 4.

In another aspect, the present invention provides a method for diagnosis, or monitoring the occurrence, development, progression or treatment, of a leukemia. The method includes the following steps: (1) generating a gene expression profile from a peripheral blood sample of a patient having the leukemia; and (2) comparing the gene expression profile to one or more reference expression profiles, wherein the gene expression profile and the one or more reference expression profiles contain the expression patterns of one or more diagnostic genes of the leukemia in peripheral blood mononuclear cells, and wherein the difference or similarity between the gene expression profile and the one or more reference expression profiles is indicative of the presence, absence, occurrence, development, progression, or effectiveness of treatment of the leukemia in the patient. In one embodiment, the leukemia is AML. “Diagnostic genes” referred to in the application include, but are not limited to, any genes that are differentially expressed in peripheral blood mononuclear cells (PBMCs) or other tissues of leukemia patients with different disease status. In particular, diagnostic genes include genes that are differentially expressed in PBMCs or other tissues of leukemia patients relative to PBMCs of leukemia-fee patients. Exemplary diagnostic genes are shown in Table 7, Table 8 and Table 9. Diagonistic genes are also referred to as disease genes in this application.

Typically, the one or more reference expression profiles include a reference expression profile representing a disease-free human. Typically, the one or more diagnostic genes include one or more genes selected from Table 7. Preferably, the one or more diagnostic genes comprise one or more genes selected from Table 8 or Table 9. In some embodiments, the one or more diagnostic genes include ten or more genes selected from Table 7. Preferably, the one or more diagnostic genes include ten or more genes selected from Table 8 or Table 9.

In another aspect, the present invention provides an array for use in a method for predicting a clinical outcome for an AML patient. The array of the invention includes a substrate having a plurality of addresses, each of which has a distinct probe disposed thereon. In some embodiments, at least 15% of the plurality of addresses have disposed thereon probes that can specifically detect prognostic genes of AML in peripheral blood mononuclear cells. In some embodiments, at least 30% of the plurality of addresses have disposed thereon probes that can specifically detect prognostic genes of AML in peripheral blood mononuclear cells. In some embodiments, at least 50% of the plurality of addresses have disposed thereon probes that can specifically detect prognostic genes of AML in peripheral blood mononuclear cells. In some embodiments, the prognostic genes are selected from Table 1, Table 2, Table 3, Table 4, Table 5 or Table 6. The probe suitable for the present invention may be a nucleic acid probe. Alternatively, the probe suitable for the invention may be an antibody probe.

In a further aspect, the present invention provides an array for use in a method for diagnosis of AML including a substrate having a plurality of addresses, each of which has a distinct probe disposed thereon. In some embodiments, at least 15% of the plurality of addresses have disposed thereon probes that can specifically detect diagnostic genes of AML in peripheral blood mononuclear cells. In some embodiments, at least 30% of the plurality of addresses have disposed thereon probes that can specifically detect diagnostic genes of AML in peripheral blood mononuclear cells. In some embodiments, at least 50% of the plurality of addresses have disposed thereon probes that can specifically detect diagnostic genes of AML in peripheral blood mononuclear cells. In some embodiments, the diagnostic genes are selected from Table 7, Table 8 or Table 9. The probe suitable for the present invention may be a nucleic acid probe. Alternatively, the probe suitable for the present invention may be an antibody probe.

In yet another aspect, the present invention provides a computer-readable medium containing a digitally-encoded expression profile having a plurality of digitally-encoded expression signals, each of which includes a value representing the expression of a prognostic gene of AML in a peripheral blood mononuclear cell. In some embodiments, each of the plurality of digitally-encoded expression signals has a value representing a prognostic gene selected from Table 1, Table 2, Table 3, Table 4, Table 5 or Table 6. In some embodiments, each of the plurality of digitally-encoded expression signals has a value representing the expression of the prognostic gene of AML in a peripheral blood mononuclear cell of a patient with a known or determinable clinical outcome. In some embodiments, the computer-readable medium of the present invention contains a digitally-encoded expression profile including at least ten digitally-encoded expression signals.

In another aspect, the present invention provides a computer-readable medium containing a digitally-encoded expression profile having a plurality of digitally-encoded expression signals, each of which has a value representing the expression of a diagnostic gene of AML in a peripheral blood mononuclear cell. In some embodiments, each of the plurality of digitally-encoded expression signals has a value representing a diagnostic gene selected from Table 7, Table 8 or Table 9. In some embodiments, each of the plurality of digitally-encoded expression signals has a value representing the expression of the diagnostic gene of AML in a peripheral blood mononuclear cell of an AML-free human. In some embodiments, the computer-readable medium of the present invention contains a digitally-encoded expression profile including at least ten digitally-encoded expression signals.

In yet another aspect, the present invention provides a kit for prognosis of a leukemia, e.g., AML. The kit includes a) one or more probes that can specifically detect prognostic genes of AML in peripheral blood mononuclear cells; and b) one or more controls, each representing a reference expression level of a prognostic gene detectable by the one or more probes. In some embodiments, the kit of the present invention includes one or more probes that can specifically detect prognostic genes selected from Table 1, Table 2, Table 3, Table 4, Table 5 or Table 6.

In another aspect, the present invention provides a kit for diagnosis of a leukemia, e.g., AML. The kit includes a) one or more probes that can specifically detect diagnostic genes of AML in peripheral blood mononuclear cells; and b) one or more controls, each representing a reference expression level of a prognostic gene detectable by the one or more probes. In some embodiments, the kit of the present invention includes one or more probes that can specifically detect diagnostic genes selected from Table 7, Table 8 or Table 9.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1A demonstrates relative PBMC expression levels of 98 class correlated genes selected from Tables 1 and 2. Among the 98 genes, 49 genes had elevated expression levels in PBMCs of patients who responded to Mylotarg combination therapy (R) relative to patients who did not respond to the therapy (NR), and the other 49 genes had elevated expression levels in PBMCs of the non-responding patients (NR) compared to the responding patients (R).

FIG. 1B shows cross validation results for each sample using a 154-gene class predictor consisting of the genes in Tables 1 and 2, where a leave-one out cross validation was performed and the prediction strengths were calculated for each sample. Samples are ordered in the same order as in FIG. 1A.

FIG. 2 illustrates an unsupervised hierarchical clustering of PBMC gene expression profiles from normal patients, patients with AML, or patients with MDS using the 7879 transcripts detected in one or more profiles with a maximal frequency greater than or equal to 10 ppm. Data were log transformed and gene expression values were median centered, and profiles were clustered using an average linkage clustering approach with an uncentered correlation similarity metric. The two main clusters of normal and non-normal are denoted as clusters 1 and 2. The subgroup in cluster 2 possessing a preponderance of AML is indicated as “AML-like” while the subgroup in cluster 2 possessing a preponderance of MDS is indicated as “MDS-like.”

FIG. 3 illustrates a gene ontology based annotation of transcripts altered during GO combination therapy of AML patients. The 52 transcripts exhibiting 3-fold or greater repression over treatment were annotated into each of the twelve categories listed. Transcripts in the immune response category were most significantly overrepresented in the group of transcripts elevated over therapy, while uncategorized transcripts were most significantly overrepresented in the group of transcripts repressed during therapy.

FIG. 4 illustrates levels of p-selectin ligand transcript in the pretreatment PBMCs of 4 AML patients who eventually experienced veno-occlusive disease (VOD) (left panel) and in pretreatment PBMCs of 32 patients who did not experience VOD (right panel). Frequency (in ppm) based on microarray analysis is plotted on the y-axis and the level of p-selectin ligand in each individual sample in each group is plotted as a discrete symbol.

FIG. 5 illustrates levels of MDR1 transcript in pretreatment PBMCs of 8 AML patients who failed to respond (NR) and in pretreatment PBMCs of 28 patients who responded (R). Frequency (in ppm) based on microarray analysis is plotted on the y-axis and the level of MDR1 transcript in each individual of the 36 pretreatment PBMC samples is indicated by each column. The p-value is based on an unpaired Student's t-test assuming unequal variances.

FIG. 6 illustrates the transcript levels of various ABC cassette transporters in PBMC samples of AML patients prior to therapy. Frequency (in ppm) based on microarray analysis is plotted on the y-axis and the average level plus standard deviation of each transporter in the NR and R groups is indicated. No significant differences in expression between NR and R were detected for any of the sequences encoding ABC transporters evaluated on U133A.

FIG. 7 illustrates levels of CD33 cell surface antigen transcript in pretreatment PBMCs of 8 patients who failed to respond (NR) and in pretreatment PBMCs of 28 patients who responded (R). Frequency (in ppm) based on microarray analysis is plotted on the y-axis and the level of CD33 transcript in each individual of the 36 pretreatment PBMC samples is indicated by each column. The p-value is based on an unpaired Student's t-test assuming unequal variances.

FIG. 8 illustrates the accuracy of a 10-gene classifier for distinguishing pretreatment PBMCs from eventual responders and eventual nonresponders to therapy. Data from baseline PBMC profiles from AML patients were scale-frequency normalized together using a total of 11382 sequences possessing at least one present call and one value of greater than or equal to 10 ppm across baseline profiles from each of two independent clinical studies involving GO-based therapy. Analyses were conducted following a z-score normalization step in Genecluster. Panel A depicts overall accuracy in a 36 member training set for models containing increasing numbers of features (transcript sequences) built using a binary classification approach with a S2N similarity metric that used median values for the class estimate. The smallest classifier (10-gene) yielding the highest overall accuracy is indicated (arrow). Panel B depicts ten-fold cross validation accuracy of the 10-gene classifier. A weighted voting algorithm was used to assign class membership using the 10-gene classifier. Confidence scores for each prediction call are indicated by columns where a downward deflection indicates a call of “NR” and an upward deflection indicates a call of “R.” True non-responders are indicated by light columns and true responders are indicated by dark columns. In this cross-validation 4/8 non-responders were correctly identified and 24/28 responders were correctly identified.

FIG. 9 illustrates the use of the 10-gene classifier to evaluate baseline PBMCs from AML patients from an independent clinical trial. The weighted voting algorithm was used to assign class membership using the 10-gene classifier. Confidence scores for each prediction call are indicated by columns where a downward deflection indicates a call of “NR” and an upward deflection indicates a call of “R.” True non-responders are indicated by light columns and true responders are indicated by dark columns. In this independent test set, 4/7 non-responders were correctly identified and 7/7 responders were correctly identified.

FIG. 10 illustrates expression levels of two genes in AML PBMCs inversely correlated with response to GO-based therapies. Panel A represents a two-dimensional plot of Affymetrix-based expression levels (in ppm) of serum/glucocorticoid regulated kinase (Y-axes) and metallothionein 1X, 1L (X-axes) in PMBC samples from AML patients. Levels of each transcript in each patient are plotted where non-responders are indicated by squares and responders are indicated by circles. The shadow indicates the area of the X-Y plot encompassing the largest number of non-responders and the smallest number of responders, defining the boundaries for this pairwise classifier. Implementing requirements for expression levels of less than 30 ppm for serum glucocorticoid regulated kinase and expression levels of greater than 30 ppm for metallothionein 1X, 1L, would have successfully identified 6/8 non-responders and only falsely identified 2 of 28 responders as non-responders in the original dataset of 36 samples. Panel B illustrates an evaluation of the 2-gene classifier in 14 AML samples from an independent clinical trial. Implementation of the same requirements correctly identified 4/7 non-responders and all responders (7/7) were also correctly identified.

### DETAILED DESCRIPTION

The present invention provides methods, reagents and systems useful for prognosis or selection of treatment of AML or other types of leukemia. These methods, reagents and systems employ leukemia prognostic genes which are differentially expressed in peripheral blood samples of leukemia patients who have different clinical outcomes. The present invention also provides methods, reagents and systems for diagnosis, or monitoring the occurrence, development, progression or treatment, of AML or other types of leukemia. These methods, reagents and systems employ diagnostic genes which are differentially expressed in peripheral blood samples of leukemia patients with different disease status. Thus, the present invention represents a significant advance in clinical pharmacogenomics and leukemia treatment.

Various aspects of the invention are described in further detail in the following subsections. The use of subsections is not meant to limit the invention. Each subsection may apply to any aspect of the invention. In this application, the use of “or” means “and/or” unless stated otherwise.

### Leukemia and Leukemia Treatment

The types of leukemia that are amenable to the present invention include, but are not limited to, acute leukemia, chronic leukemia, lymphocytic leukemia, or nonlymphocytic leukemia (e.g., myelogenous, monocytic, or erythroid). Acute leukemia includes, for example, AML or ALL (acute lymphoblastic leukemia). Chronic leukemia includes, for example, CML (chronic myelogenous leukemia), CLL (chronic lymphocytic leukemia), or hairy cell leukemia. The present invention also contemplates genes that are prognostic of clinical outcome of patients having myelodysplastic syndromes (MDS).

Any leukemia treatment regime can be analyzed according to the present invention. Examples of these leukemia treatments include, but are not limited to, chemotherapy, drug therapy, gene therapy, immunotherapy, biological therapy, radiation therapy, bone marrow transplantation, surgery, or a combination thereof. Other conventional, non-conventional, novel or experimental therapies, including treatments under clinical trials, can also be evaluated according to the present invention.

A variety of anti-cancer agents can be used to treat leukemia. Examples of these agents include, but are not limited to, alkylators, anthracyclines, antibiotics, biphosphonates, folate antagonists, inorganic arsenates, microtubule inhibitors, nitrosoureas, nucleoside analogs, retinoids, or topoisomerase inhibitors.

Examples of alkylators include, but are not limited to, busulfan (Myleran, Busulfex), chlorambucil (Leukeran), cyclophosphamide (Cytoxan, Neosar), melphalan, L-PAM (Alkeran), dacarbazine (DTIC-Dome), and temozolamide (Temodar). Examples of anthracyclines include, but are not limited to, doxorubicin (Adriamycin, Doxil, Rubex), mitoxantrone (Novantrone), idarubicin (Idamycin), valrubicin (Valstar), and epirubicin (Ellence). Examples of antibiotics include, but are not limited to, dactinomycin, actinomycin D (Cosmegen), bleomycin (Blenoxane), and daunorubicin, daunomycin (Cerubidine, DanuoXome). Examples of biphosphonate inhibitors include, but are not limited to, zoledronate (Zometa). Examples of folate antagonists include, but are not limited to, methotrexate and tremetrexate. Examples of inorganic arsenates include, but are not limited to, arsenic trioxide (Trisenox). Examples of microtubule inhibitors, which may inhibit either microtubule assembly or disassembly, include, but are not limited to, vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. Examples of nitrosoureas include, but are not limited to, procarbazine (Matulane), lomustine, CCNU (CeeBU), carmustine (BCNU, BiCNU, Gliadel Wafer), and estramustine (Emcyt). Examples of nucleoside analogs include, but are not limited to, mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda). Examples of retinoids include, but are not limited to, tretinoin, ATRA (Vesanoid), alitretinoin (Panretin), and bexarotene (Targretin). Examples of topoisomerase inhibitors include, but are not limited to, etoposide, VP-16 (Vepesid), teniposide, VM-26 (Vumon), etoposide phosphate (Etopophos), topotecan (Hycamtin), and irinotecan (Camptostar). Therapies including the use of any of these anti-cancer agents can be evaluated according to the present invention.

Leukemia can also be treated by antibodies that specifically recognize diseased or otherwise unwanted cells. Antibodies suitable for this purpose include, but are not limited to, polyclonal, monoclonal, mono-specific, poly-specific, humanized, human, single-chain, chimeric, synthetic, recombinant, hybrid, mutated, grafted, or in vitro generated antibodies. Suitable antibodies can also be Fab, F(ab′)₂, Fv, scFv, Fd, dAb, or other antibody fragments that retain the antigen-binding function. In many cases, an antibody employed in the present invention can bind to a specific antigen on the diseased or unwanted cells (e.g., the CD33 antigen on myeloblasts or myeloid progenitor cells) with a binding affinity of at least 10⁻⁶ M⁻¹, 10⁻⁷ M⁻¹, 10⁻⁸ M⁻¹, 10⁻⁹ M⁻¹, or stronger.

Many antibodies employed in the present invention are conjugated with a cytotoxic or otherwise anticellular agent which can kill or suppress the growth or division of cells. Examples of cytotoxic or anticellular agents include, but are not limited to, the anti-neoplastic agents described above, and other chemotherapeutic agents, radioisotopes or cytotoxins. Two or more different cytotoxic moieties can be coupled to one antibody, thereby accommodating variable or even enhanced anti-cancer activities.

Linking or coupling one or more cytotoxic moieties to an antibody may be achieved by a variety of mechanisms, for example, covalent binding, affinity binding, intercalation, coordinate binding and complexation. Preferred binding methods are those involving covalent binding, such as using chemical cross-linkers, natural peptides or disulfide bonds.

Covalent binding can be achieved, for example, by direct condensation of existing side chains or by the incorporation of external bridging molecules. Many bivalent or polyvalent agents are useful in coupling protein molecules to other proteins, peptides or amine functions. Examples of coupling agents are, without limitation, carbodiimides, diisocyanates, glutaraldehyde, diazobenzenes, and hexamethylene diamines.

In one embodiment, an antibody employed in the present invention is first derivatized before being attaching with a cytotoxic moiety. “Derivatize” means chemical modification(s) of the antibody substrate with a suitable cross-linking agent. Examples of cross-linking agents for use in this manner include the disulfide-bond containing linkers SPDP (N-succinimidyl-3-(2-pyridyldithio)propionate) and SMPT (4-succinimidyl-oxycarbonyl-α-methyl-α(2-pyridyldithio)toluene). Biologically releasable bonds can also be used to construct a clinically active antibody, such that a cytotoxic moiety can be released from the antibody once it binds to or enters the target cell. Numerous types of linking constructs are known for this purpose (e.g., disulfide linkages).

Anti-neoplastic agent(s) employed in a leukemia treatment regime can be administered via any common route so long as the target tissue or cell is available via that route. This includes, but is not limited to, intravenous, catheterization, orthotopic, intradermal, subcutaneous, intramuscular, intraperitoneal intrtumoral, oral, nasal, buccal, rectal, vaginal, or topical administration. Selection of anti-neoplastic agents and dosage regimes may depend on various factors, such as the drug combination employed, the particular disease being treated, and the condition and prior history of the patient. Specific dose regimens for known and approved anti-neoplastic agents can be found in the current version of Physician's Desk Reference, Medical Economics Company, Inc., Oradell, N.J.

In addition, a leukemia treatment regime can include a combination of different types of therapies, such as chemotherapy plus antibody therapy. The present invention contemplates identification of prognostic genes for all types of leukemia treatment regime.

In one aspect, the present invention features identification of genes that are prognostic of clinical outcome of AML patients who undergo an anti-cancer treatment. An AML treatment can include a remission induction therapy, a postremission therapy, or a combination thereof. The purpose of the remission induction therapy is to attain remission by killing the leukemia cells in the blood or bone marrow. The purpose of the postremission therapy is to maintain remission by killing any remaining leukemia cells that may not be active but could begin to regrow and cause a relapse.

Standard remission induction therapies for AML patients include, but are not limited to, combination chemotherapy, stem cell transplantation, high-dose combination chemotherapy, all-trans retinoic acid (ATRA) plus chemotherapy, or intrathecal chemotherapy. Standard postremission therapies include, but are not limited to, combination chemotherapy, high-dose chemotherapy and stem cell transplantation using donor stem cells, or high-dose chemotherapy and stem cell transplantation using the patient's stem cells with or without radiation therapy. For recurrent AML patients, standard treatments include, but are not limited to, combination chemotherapy, biologic therapy with monoclonal antibodies, stem cell transplantation, low dose radiation therapy as palliative therapy to relieve symptoms and improve quality of life, or arsenic trioxide therapy. Nonstandard therapies, including treatments under clinical trials, are also contemplated by the present invention.

In many embodiments, the treatment regimes described in U.S. Patent Application Publication No. 20040152632 are employed to treat AML or MDS. Genes prognostic of patient outcome under these treatment regimes can be identified according to the present invention. In one example, the treatment regime includes administration of at least one chemotherapy drug and an anti-CD33 antibody conjugated with a cytotoxic agent. The chemotherapy drug can be selected, without limitation, from the group consisting of an anthracycline and a pyrimidine or purine nucleoside analog. The cytotoxic agent can be, for example, a calicheamicin or an esperamicin.

Anthracyclines suitable for treating AML or MDS include, but are not limited to, doxorubicin, daunorubicin, idarubicin, aclarubicin, zorubicin, mitoxantrone, epirubicin, carubicin, nogalamycin, menogaril, pitarubicin, and valrubicin. Pyrimidine or purine nucleoside analogs useful for treating AML or MDS include, but are not limited to, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, or tubercidin. Other anthracyclines and pyrimidine/purine nucleoside analogs can also be used in the present invention.

In a further example, the AML/MDS treatment regime includes administration of gemtuzumab ozogamicin (GO), daunorubicin and cytarabine to a patient in need of the treatment. Gemtuzumab ozogamicin can be administered, without limitation, in an amount of about 3 mg/m² to about 9 mg/m² per day, such as about 3, 4, 5, 6, 7, 8 or 9 mg/m² per day. Daunorubicin can be administered, for example, in an amount of about 45 mg/m² to about 60 mg/m² per day, such as about 45, 50, 55 or 60 mg/m² per day. Cytarabine can be administered, without limitation, in an amount of about 100 mg/m² to about 200 mg/m² per day, such as about 100, 125, 150, 175 or 200 mg/m² per day. In one example, the daunorubicin employed in the treatment regime is daunorubicin hydrochloride.

### Clinical Outcome

Clinical outcome of leukemia patients can be assessed by a number of criteria. Examples of clinical outcome measures include, but are not limited to, complete remission, partial remission, non-remission, survival, development of adverse events, or any combination thereof. Patients with complete remission show less than 5% blast cells in the bone marrow after the treatment. Patients with partial remission exhibit a decrease in the blast percentage to certain degree but do not achieve normal hematopoiesis with less than 5% blast cells. The blast percentage in the bone marrow of non-remission patients does not decrease in a significant way in response to the treatment.

In many cases, the peripheral blood samples used for the identification of the prognostic genes are “baseline” or “pretreatment” samples. These samples are isolated from respective leukemia patients prior to a therapeutic treatment and can be used to identify genes whose baseline peripheral blood expression profiles are correlated with clinical outcome of these leukemia patients in response to the treatment. Peripheral blood samples isolated at other treatment or disease stages can also be used to identify leukemia prognostic genes.

A variety of types of peripheral blood samples can be used in the present invention. In one embodiment, the peripheral blood samples are whole blood samples. In another embodiment, the peripheral blood samples comprise enriched PBMCs. By “enriched,” it means that the percentage of PBMCs in the sample is higher than that in whole blood. In some cases, the PBMC percentage in an enriched sample is at least 1, 2, 3, 4, 5 or more times higher than that in whole blood. In some other cases, the PBMC percentage in an enriched sample is at least 90%, 95%, 98%, 99%, 99.5%, or more. Blood samples containing enriched PBMCs can be prepared using any method known in the art, such as Ficoll gradients centrifugation or CPTs (cell purification tubes).

### Gene Expression Analysis

The relationship between peripheral blood gene expression profiles and patient outcome can be evaluated by using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as PBMCs from a patient in a selected outcome class, are hybridized to the probes on a nucleic acid array. Signals detected after the formation of hybridization complexes correlate to the polynucleotide levels in the sample. In the differential hybridization format, polynucleotides derived from two biological samples, such as one from a patient in a first outcome class and the other from a patient in a second outcome class, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

### Correlation Analysis

The gene expression data collected from nucleic acid arrays can be correlated with clinical outcome using a variety of methods. Methods suitable for this purpose include, but are not limited to, statistical methods (such as Spearman's rank correlation, Cox proportional hazard regression model, ANOVA/t test, or other rank tests or survival models) and class-based correlation metrics (such as nearest-neighbor analysis).

In one embodiment, patients with a specified leukemia (e.g., AML) are divided into at least two classes based on their responses to a therapeutic treatment. The correlation between peripheral blood gene expression (e.g., PBMC gene expression) and the patient outcome classes is then analyzed by a supervised cluster or learning algorithm. Supervised algorithms suitable for this purpose include, but are not limited to, nearest-neighbor analysis, support vector machines, the SAM method, artificial neural networks, and SPLASH. Under a supervised analysis, clinical outcome of each patient is either known or determinable. Genes that are differentially expressed in peripheral blood cells (e.g., PBMCs) of one class of patients relative to another class of patients can be identified. These genes can be used as surrogate markers for predicting clinical outcome of a leukemia patient of interest. Many of the genes thus identified are correlated with a class distinction that represents an idealized expression pattern of these genes in patients of different outcome classes.

In another embodiment, patients with a specified leukemia (e.g., AML) can be divided into at least two classes based on their peripheral blood gene expression profiles. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of patients in one class may have a first clinical outcome, and a substantial number of patients in another class may have a second clinical outcome. Genes that are differentially expressed in the peripheral blood cells of one class of patients relative to another class of patients can be identified. These genes can also be used as prognostic markers for predicting clinical outcome of a leukemia patient of interest.

In yet another embodiment, patients with a specified leukemia (e.g., AML) can be divided into three or more classes based on their clinical outcomes or peripheral blood gene expression profiles. Multi-class correlation metrics can be employed to identify genes that are differentially expressed in one class of patients relative to another class. Exemplary multi-class correlation metrics include, but are not limited to, those employed by GeneCluster 2 software provided by MIT Center for Genome Research at Whitehead Institute (Cambridge, Mass.).

In a further embodiment, nearest-neighbor analysis (also known as neighborhood analysis) is used to correlate peripheral blood gene expression profiles with clinical outcome of leukemia patients. The algorithm for neighborhood analysis is described in Golub, et al., SCIENCE, 286: 531-537 (1999); Slonim, et al., PROCS. OF THE FOURTH ANNUAL INTERNATIONAL CONFERENCE ON COMPUTATIONAL MOLECULAR BIOLOGY, Tokyo, Japan, April 8-11, p 263-272 (2000); and U.S. Pat. No. 6,647,341. Under one version of the neighborhood analysis, the expression profile of each gene can be represented by an expression vector g=(e₁, e₂, e₃, . . . , eₙ), where eᵢ corresponds to the expression level of gene “g” in the ith sample. A class distinction can be represented by an idealized expression pattern c=(c₁, c₂, c₃, . . . , cₙ), where cᵢ=1 or −1, depending on whether the ith sample is isolated from class 0 or class 1. Class 0 may include patients having a first clinical outcome, and class 1 includes patients having a second clinical outcome. Other forms of class distinction can also be employed. Typically, a class distinction represents an idealized expression pattern, where the expression level of a gene is uniformly high for samples in one class and uniformly low for samples in the other class.

The correlation between gene “g” and the class distinction can be measured by a signal-to-noise score:

P(g,c)=[μ₁(g)−μ₂(g)]/[σ₁(g)+σ₂(g)]

where μ₁(g) and μ₂(g) represent the means of the log-transformed expression levels of gene “g” in class 0 and class 1, respectively, and σ₁(g) and σ₂(g) represent the standard deviation of the log-transformed expression levels of gene “g” in class 0 and class 1, respectively. A higher absolute value of a signal-to-noise score indicates that the gene is more highly expressed in one class than in the other. In one example, the samples used to derive the signal-to-noise scores comprise enriched or purified PBMCs and, therefore, the signal-to-noise score P(g,c) represents a correlation between the class distinction and the expression level of gene “g” in PBMCs.

The correlation between gene “g” and the class distinction can also be measured by other methods, such as by the Pearson correlation coefficient or the Euclidean distance, as appreciated by those skilled in the art.

The significance of the correlation between peripheral blood gene expression profiles and the class distinction can be evaluated using a random permutation test. An unusually high density of genes within the neighborhoods of the class distinction, as compared to random patterns, suggests that many genes have expression patterns that are significantly correlated with the class distinction. The correlation between genes and the class distinction can be diagrammatically viewed through a neighborhood analysis plot, in which the y-axis represents the number of genes within various neighborhoods around the class distinction and the x-axis indicates the size of the neighborhood (i.e., P(g,c)). Curves showing different significance levels for the number of genes within corresponding neighborhoods of randomly permuted class distinctions can also be included in the plot.

In many embodiments, the prognostic genes employed in the present invention are above the median significance level in the neighborhood analysis plot. This means that the correlation measure P(g,c) for each prognostic gene is such that the number of genes within the neighborhood of the class distinction having the size of P(g,c) is greater than the number of genes within the corresponding neighborhoods of randomly permuted class distinctions at the median significance level. In many other embodiments, the prognostic genes employed in the present invention are above the 40%, 30%, 20%, 10%, 5%, 2%, or 1% significance level. As used herein, x % significance level means that x % of random neighborhoods contain as many genes as the real neighborhood around the class distinction.

Class predictors can be constructed using the prognostic genes of the present invention. These class predictors can be used to assign a leukemia patient of interest to an outcome class. In one embodiment, the prognostic genes employed in a class predictor are limited to those shown to be significantly correlated with a class distinction by the permutation test, such as those at above the 1%, 2%, 5%, 10%, 20%, 30%, 40%, or 50% significance level. In another embodiment, the PBMC expression level of each prognostic gene in a class predictor is substantially higher or substantially lower in one class of patients than in another class of patients. In still another embodiment, the prognostic genes in a class predictor have top absolute values of P(g,c). In yet another embodiment, the p-value under a Student's t-test (e.g., two-tailed distribution, two sample unequal variance) for each prognostic gene in a class predictor is no more than 0.05, 0.01, 0.005, 0.001, 0.0005, 0.0001, or less. For each prognostic gene, the p-value suggests the statistical significance of the difference observed between the average PBMC expression profiles of the gene in one class of patients versus another class of patients. Lesser p-values indicate more statistical significance for the differences observed between different classes of leukemia patients.

The SAM method can also be used to correlate peripheral blood gene expression profiles with different outcome classes. The prediction analysis of microarrays (PAM) method can then be used to identify class predictors that can best characterize a predefined outcome class and predict the class membership of new samples. See Tibshirani, et al., PROC. NATL. ACAD. SCI. U.S.A., 99:6567-6572 (2002).

In many embodiments, a class predictor of the present invention has high prediction accuracy under leave-one-out cross validation, 10-fold cross validation, or 4-fold cross validation. For instance, a class predictor of the present invention can have at least 50%, 60%, 70%, 80%, 90%, 95%, or 99% accuracy under leave-one-out cross validation, 10-fold cross validation, or 4-fold cross validation. In a typical k-fold cross validation, the data is divided into k subsets of approximately equal size. The model is trained k times, each time leaving out one of the subsets from training and using the omitted subset as the test samples to calculate the prediction error. If k equals the sample size, it becomes the leave-one-out cross validation.

Other class-based correlation metrics or statistical methods can also be used to identify prognostic genes whose expression profiles in peripheral blood samples are correlated with clinical outcome of leukemia patients. Many of these methods can be performed by using commercial or publicly accessible softwares.

Other methods capable of identifying leukemia prognostic genes include, but are not limited, RT-PCR, Northern Blot, in situ hybridization, and immunoassays such as ELISA, RIA or Western Blot. These genes are differentially expressed in peripheral blood cells (e.g., PBMCs) of one class of patients relative to another class of patients. In many cases, the average peripheral blood expression level of each of these genes in one class of patients is statistically different from that in another class of patients. For instance, the p-value under an appropriate statistical significance test (e.g., Student's t-test) for the observed difference can be no more than 0.05, 0.01, 0.005, 0.001, 0.0005, 0.0001, or less. In many other cases, each prognostic gene thus identified has at least 2-, 3-, 4-, 5-, 10-, or 20-fold difference in the average PBMC expression level between one class of patients and another class of patients.

### Identification of AML Prognostic Genes Using HG-U133A Microarrays

As an example, the present invention characterized signatures in peripheral blood of AML patients that are indicative of remission in response to a chemotherapy regimen consisting of daunorubicin and cytarabine induction therapy with concomitant administration of GO. In particular, the present invention employed a pharmacogenomic approach to identify transcriptional patterns in peripheral blood samples taken from AML patients prior to treatment that were correlated with positive response to the therapy regimen.

Of the 36 AML patients who consented for pharmacogenomic analysis, 28 achieved a positive response and 8 failed to respond to the treatment regimen following 36 days of induction therapy. Genecluster's default correlation metric (Golub, et al., SCIENCE, 286: 531-537 (1999)) was used to identify genes with expression levels highly correlated with responder and non-responder profiles in the entire set of samples. The low number of non-responders in the pharmacogenomic consented patients precluded division of the pretreatment blood samples into a training and test set. Therefore all samples were used to identify gene classifiers that displayed high accuracies for classification of responder samples versus non-responder samples.

Table 1 lists genes which had higher pretreatment PBMC expression levels in AML patients who eventually failed to respond to the GO combination chemotherapy (non-remission or partial remission), compared to AML patients who responded to the therapy (remission to less than 5% blasts). Genes showing greatest fold elevation in non-responding patients at baseline PBMCs are listed in Table 3. Table 2 describes transcripts that had higher pretreatment expression levels in PBMCs of AML patients who eventually respond to the GO combination chemotherapy, compared to AML patients who did not respond to the therapy. Genes showing greatest fold elevation in responding patients at baseline PBMCs are listed in Table 4. “Fold Change (NR/R)” denotes the ratio of the mean expression level of a gene in PBMCs of non-responding AML patients over that in responding AML patients. “Fold Change (R/NR)” represents the ratio of the mean expression level of a gene in PBMCs of responding AML patients over that in non-responding AML patients. In each table, the transcripts are presented in order of the signal to noise metric score calculated by the supervised algorithm described in Examples. Each gene depicted in Tables 1-4 and the corresponding unigene(s) were identified according to Affymetrix annotations.

Classifiers consisting of genes selected from Tables 1 and 2 were built and evaluated for class prediction accuracy. Each classifier included the top n gene(s) in Table 1 and the top n gene(s) in Table 2, where n represents an integer no less than 1. For example, a first classifier being evaluated included Gene Nos. 1 and 78, a second classifier included Gene Nos. 1-2 and 78-79, a third classifier included Gene Nos. 1-3 and 78-80, a fourth classifier included Gene Nos. 1-4 and 78-81, and so on. Each classifier thus constructed produced significant prediction accuracy. For instance, a classifier consisting of all of the 154 genes in Tables 1 and 2 yielded 81% overall prediction accuracy by 4-fold cross validation on the peripheral blood profiles used in the present study.

Correlation analysis between the pretreatment transcriptional patterns and the clinical outcomes, including occurrence of adverse events, are further discussed in Examples. Additional classifiers are also disclosed in Examples.

TABLE 1
Genes Having Higher Baseline Peripheral Blood Expression
Levels in Non-Responding Patients

| | | SEQ | | Fold | | |
|---|---|---|---|---|---|---|
| Gene | | ID | Unigene | Change | Gene | |
| No. | Qualifier | NO: | No. | (NR/R) | Symbol | Gene Name |
| 1 | 208581_x_at | 1 | Hs.278462 | 2.04 | MT1L, | metallothionein 1L, metallothionein |
| | | | | | MT1X | 1X |
| 2 | 208963_x_at | 2 | Hs.132898 | 1.34 | FADS1 | fatty acid desaturase 1 |
| 3 | 216336_x_at | 3 | | 1.73 | | unknown |
| 4 | 209407_s_at | 4 | Hs.6574 | 1.88 | DEAF1 | deformed epidermal autoregulatory |
| | | | | | | factor 1 (Drosophila) |
| 5 | 203725_at | 5 | Hs.80409 | 1.84 | GADD45A | growth arrest and DNA-damage- |
| | | | | | | inducible, alpha |
| 6 | 205366_s_at | 6 | Hs.98428 | 1.69 | HOXB6 | homeo box B6 |
| 7 | 209480_at | 7 | Hs.73931 | 1.61 | HLA-DQB1 | major histocompatibility complex, |
| | | | | | | class II, DQ beta 1 |
| 8 | 204430_s_at | 8 | Hs.33084 | 1.61 | SLC2A5 | solute carrier family 2 (facilitated |
| | | | | | | glucose/fructose transporter), |
| | | | | | | member 5 |
| 9 | 204468_s_at | 9 | Hs.78824 | 3.62 | TIE | tyrosine kinase with immunoglobulin |
| | | | | | | and epidermal growth factor |
| | | | | | | homology domains |
| 10 | 212747_at | 10 | Hs.20060 | 1.10 | KIAA0229 | KIAA0229 protein |
| 11 | 205227_at | 11 | Hs.173880 | 1.88 | IL1RAP | interleukin 1 receptor accessory |
| | | | | | | protein |
| 12 | 201539_s_at | 12 | Hs.239069 | 1.09 | FHL1 | four and a half LIM domains 1 |
| 13 | 203373_at | 13 | Hs.110776 | 2.94 | STATI2 | STAT induced STAT inhibitor-2 |
| 14 | 210093_s_at | 14 | Hs.57904 | 1.52 | MAGOH | mago-nashi homolog, proliferation- |
| | | | | | | associated (Drosophila) |
| 15 | 209392_at | 15 | Hs.174185 | 2.64 | ENPP2 | ectonucleotide |
| | | | | | | pyrophosphatase/phosphodiesterase |
| | | | | | | 2 (autotaxin) |
| 16 | 203372_s_at | 16 | Hs.110776 | 2.44 | STATI2 | STAT induced STAT inhibitor-2 |
| 17 | 212813_at | 17 | Hs.334703 | 1.48 | FLJ14529 | hypothetical protein FLJ14529 |
| 18 | 204326_x_at | 18 | Hs.199263 | 1.78 | MT1L, | metallothionein 1L, metallothionein |
| | | | | | MT1X, | 1X, serine threonine kinase 39 |
| | | | | | STK39 | (STE20/SPS1 homolog, yeast) |
| 19 | 203177_x_at | 19 | Hs.75133 | 1.39 | TFAM | transcription factor A, mitochondrial |
| 20 | 212173_at | 20 | Hs.171811 | 1.61 | AK2 | adenylate kinase 2 |
| 21 | 204438_at | 21 | Hs.75182 | 2.26 | MRC1 | mannose receptor, C type 1 |
| 22 | 212185_x_at | 22 | Hs.118786 | 1.89 | MT2A | metallothionein 2A |
| 23 | 214281_s_at | 23 | Hs.48297 | 1.56 | ZNF363 | zinc finger protein 363 |
| 24 | 217975_at | 24 | Hs.15984 | 1.65 | LOC51186 | pp21 homolog |
| 25 | 220974_x_at | 25 | Hs.283844 | 2.10 | BA108L7.2 | similar to rat tricarboxylate carrier- |
| | | | | | | like protein |
| 26 | 218807_at | 26 | Hs.267659 | 1.52 | VAV3 | vav 3 oncogene |
| 27 | 201263_at | 27 | Hs.84131 | 1.43 | TARS | threonyl-tRNA synthetase |
| 28 | 217165_x_at | 28 | n/a | 2.02 | | unknown |
| 29 | 201013_s_at | 29 | Hs.117950 | 1.54 | PAICS | phosphoribosylaminoimidazole |
| | | | | | | carboxylase, |
| | | | | | | phosphoribosylaminoimidazole |
| | | | | | | succinocarboxamide synthetase |
| 30 | 208835_s_at | 30 | Hs.3688 | 1.46 | LUC7A | cisplatin resistance-associated |
| | | | | | | overexpressed protein |
| 31 | 218049_s_at | 31 | Hs.333823 | 1.48 | MRPL13 | mitochondrial ribosomal protein L13 |
| 32 | 217824_at | 32 | Hs.184325 | 1.25 | NCUBE1 | non-canonical ubquitin conjugating |
| | | | | | | enzyme 1 |
| 33 | 220059_at | 33 | Hs.121128 | 1.56 | BRDG1 | BCR downstream signaling 1 |
| 34 | 202942_at | 34 | Hs.74047 | 1.78 | ETFB | electron-transfer-flavoprotein, beta |
| | | | | | | polypeptide |
| 35 | 200986_at | 35 | Hs.151242 | 1.38 | SERPING1 | serine (or cysteine) proteinase |
| | | | | | | inhibitor, clade G (C1 inhibitor), |
| | | | | | | member 1, (angioedema, hereditary) |
| 36 | 221652_s_at | 36 | Hs.22595 | 1.33 | FLJ10637 | hypothetical protein FLJ10637 |
| 37 | 211456_x_at | 37 | Hs.367850 | 1.75 | | unknown |
| 38 | 201487_at | 38 | Hs.10029 | 1.74 | CTSC | cathepsin C |
| 39 | 220668_s_at | 39 | Hs.251673 | 2.00 | DNMT3B | DNA (cytosine-5-)-methyltransferase |
| | | | | | | 3 beta |
| 40 | 215088_s_at | 40 | Hs.355964 | 1.43 | SDHC | succinate dehydrogenase complex, |
| | | | | | | subunit C, integral membrane |
| | | | | | | protein, 15 kD |
| 41 | 205394_at | 41 | Hs.20295 | 1.07 | CHEK1 | CHK1 checkpoint homolog (S. pombe) |
| 42 | 218364_at | 42 | Hs.57672 | 1.38 | LRRFIP2 | leucine rich repeat (in FLII) |
| | | | | | | interacting protein 2 |
| 43 | 222010_at | 43 | Hs.4112 | 1.27 | TCP1 | t-complex 1 |
| 44 | 218286_s_at | 44 | Hs.14084 | 1.47 | RNF7 | ring finger protein 7 |
| 45 | 208955_at | 45 | Hs.367676 | 1.21 | DUT | dUTP pyrophosphatase |
| 46 | 210715_s_at | 46 | Hs.31439 | 2.04 | SPINT2 | serine protease inhibitor, Kunitz |
| | | | | | | type, 2 |
| 47 | 218055_s_at | 47 | Hs.16470 | 1.21 | FLJ10904 | hypothetical protein FLJ10904 |
| 48 | 202946_s_at | 48 | Hs.7935 | 2.65 | BTBD3 | BTB (POZ) domain containing 3 |
| 49 | 201397_at | 49 | Hs.3343 | 1.14 | PHGDH | phosphoglycerate dehydrogenase |
| 50 | 204050_s_at | 50 | Hs.104143 | 1.54 | CLTA | clathrin, light polypeptide (Lca) |
| 51 | 201425_at | 51 | Hs.195432 | 2.29 | ALDH2 | aldehyde dehydrogenase 2 family |
| | | | | | | (mitochondrial) |
| 52 | 204484_at | 52 | Hs.132463 | 1.58 | PIK3C2B | phosphoinositide-3-kinase, class 2, |
| | | | | | | beta polypeptide |
| 53 | 212072_s_at | 53 | n/a | 1.40 | | unknown |
| 54 | 215905_s_at | 54 | Hs.10290 | 1.34 | HPRP8BP | U5 snRNP-specific 40 kDa protein |
| | | | | | | (hPrp8-binding) |
| 55 | 201827_at | 55 | Hs.250581 | 1.47 | SMARCD2 | SWI/SNF related, matrix associated, |
| | | | | | | actin dependent regulator of |
| | | | | | | chromatin, subfamily d, member 2 |
| 56 | 211031_s_at | 56 | Hs.104717 | 1.21 | CYLN2 | cytoplasmic linker 2 |
| 57 | 217963_s_at | 57 | Hs.169248 | 2.49 | HCS, | cytochrome c, nerve growth factor |
| | | | | | NGFRAP1 | receptor (TNFRSF16) associated |
| | | | | | | protein 1 |
| 58 | 208029_s_at | 58 | Hs.296398 | 6.87 | LC27 | putative integral membrane |
| | | | | | | transporter |
| 59 | 202184_s_at | 59 | Hs.12457 | 1.37 | NUP133 | nucleoporin 133 kD |
| 60 | 214228_x_at | 60 | Hs.129780 | 2.36 | TNFRSF4 | tumor necrosis factor receptor |
| | | | | | | superfamily, member 4 |
| 61 | 214113_s_at | 61 | Hs.10283 | 1.42 | RBM8A | RNA binding motif protein 8A |
| 62 | 217957_at | 62 | Hs.279818 | 1.26 | AF093680 | similar to mouse Glt3 or D. malanogaster |
| | | | | | | transcription factor IIB |
| 63 | 218622_at | 63 | Hs.5152 | 1.30 | MGC5585 | hypothetical protein MGC5585 |
| 64 | 208937_s_at | 64 | Hs.75424 | 1.20 | ID1 | inhibitor of DNA binding 1, dominant |
| | | | | | | negative helix-loop-helix protein |
| 65 | 213258_at | 65 | Hs.288582 | 1.94 | | unknown |
| 66 | 206480_at | 66 | Hs.456 | 2.05 | LTC4S | leukotriene C4 synthase |
| 67 | 203405_at | 67 | Hs.5198 | 1.47 | DSCR2 | Down syndrome critical region gene 2 |
| 68 | 202430_s_at | 68 | Hs.198282 | 1.50 | PLSCR1 | phospholipid scramblase 1 |
| 69 | 218289_s_at | 69 | Hs.170737 | 1.23 | FLJ23251 | hypothetical protein FLJ23251 |
| 70 | 209757_s_at | 70 | Hs.25960 | 1.36 | MYCN | v-myc myelocytomatosis viral related |
| | | | | | | oncogene, neuroblastoma derived |
| | | | | | | (avian) |
| 71 | 210298_x_at | 71 | Hs.239069 | 1.14 | FHL1 | four and a half LIM domains 1 |
| 72 | 217814_at | 72 | Hs.8207 | 1.50 | GK001 | GK001 protein |
| 73 | 201690_s_at | 73 | Hs.2384 | 1.63 | TPD52 | tumor protein D52 |
| 74 | 201923_at | 74 | Hs.83383 | 1.18 | PRDX4 | peroxiredoxin 4 |
| 75 | 210665_at | 75 | Hs.170279 | 1.81 | TFPI | tissue factor pathway inhibitor |
| | | | | | | (lipoprotein-associated coagulation |
| | | | | | | inhibitor) |
| 76 | 212859_x_at | 76 | Hs.74170 | 1.47 | | unknown |
| 77 | 221504_s_at | 77 | Hs.19575 | 1.60 | ATP6V1H | ATPase, H+ transporting, lysosomal |
| | | | | | | 50/57 kD V1 subunit H |

TABLE 2
Genes Having Higher Baseline Peripheral Blood Expression
Levels in Responding Patients

| | | | | Fold | | |
|---|---|---|---|---|---|---|
| Gene | | SEQ | | Change | | |
| No. | Qualifier | ID NO: | Unigene No. | (R/NR) | Gene Symbol | Gene Name |
| 78 | 203739_at | 78 | Hs.155040 | 1.50 | ZNF217 | zinc finger protein 217 |
| 79 | 219593_at | 79 | Hs.237856 | 3.57 | PHT2 | peptide transporter 3 |
| 80 | 204132_s_at | 80 | Hs.14845 | 1.93 | FOXO3A | forkhead box O3A |
| 81 | 210972_x_at | 81 | Hs.74647 | 3.89 | TRA@ | T cell receptor alpha locus |
| 82 | 205220_at | 82 | Hs.137555 | 3.11 | HM74 | putative chemokine receptor; |
| | | | | | | GTP-binding protein |
| 83 | 201235_s_at | 83 | Hs.75462 | 2.35 | BTG2 | BTG family, member 2 |
| 84 | 209535_s_at | 84 | Hs.301946 | 1.69 | LBC | lymphoid blast crisis |
| | | | | | | oncogene |
| 85 | 209671_x_at | 85 | Hs.74647 | 3.95 | TRA@ | T cell receptor alpha locus |
| 86 | 203945_at | 86 | Hs.172851 | 1.62 | ARG2 | arginase, type II |
| 87 | 219434_at | 87 | Hs.283022 | 2.61 | TREM1 | triggering receptor expressed |
| | | | | | | on myeloid cells 1 |
| 88 | 221558_s_at | 88 | Hs.44865 | 2.63 | LEF1 | lymphoid enhancer-binding |
| | | | | | | factor 1 |
| 89 | 214056_at | 89 | Hs.86386 | 1.91 | MCL1 | myeloid cell leukemia |
| | | | | | | sequence 1 (BCL2-related) |
| 90 | 203907_s_at | 90 | Hs.4764 | 2.63 | KIAA0763 | KIAA0763 gene product |
| 91 | 217022_s_at | 91 | Hs.293441 | 2.00 | | unknown |
| 92 | 203413_at | 92 | Hs.79389 | 2.04 | NELL2 | NEL-like 2 (chicken) |
| 93 | 212074_at | 93 | Hs.7531 | 1.62 | KIAA0810 | KIAA0810 protein |
| 94 | 220987_s_at | 94 | Hs.172012 | 1.62 | DKFZP434J037 | hypothetical protein |
| | | | | | | DKFZp434J037 |
| 95 | 212658_at | 95 | Hs.79299 | 1.66 | LHFPL2 | lipoma HMGIC fusion |
| | | | | | | partner-like 2 |
| 96 | 214467_at | 96 | Hs.131924 | 2.14 | GPR65 | G protein-coupled receptor |
| | | | | | | 65 |
| 97 | AFFX-DapX- | 97 | n/a | 1.34 | | unknown |
| | 3_at | | | | | |
| 98 | 212812_at | 98 | Hs.288232 | 2.39 | | unknown |
| 99 | 212579_at | 99 | Hs.8118 | 1.83 | KIAA0650 | KIAA0650 protein |
| 100 | 206133_at | 100 | Hs.139262 | 1.86 | HSXIAPAF1 | XIAP associated factor-1 |
| 101 | 213797_at | 101 | Hs.17518 | 1.80 | cig5 | vipirin |
| 102 | 213958_at | 102 | Hs.81226 | 1.55 | CD6 | CD6 antigen |
| 103 | 204638_at | 103 | Hs.1211 | 1.66 | ACP5 | acid phosphatase 5, tartrate |
| | | | | | | resistant |
| 104 | 202481_at | 104 | Hs.17144 | 1.69 | SDR1 | short-chain |
| | | | | | | dehydrogenase/reductase 1 |
| 105 | 204961_s_at | 105 | Hs.1583 | 1.95 | NCF1 | neutrophil cytosolic factor 1 |
| | | | | | | (47 kD, chronic |
| | | | | | | granulomatous disease, |
| | | | | | | autosomal 1) |
| 106 | 209448_at | 106 | Hs.90753 | 1.36 | HTATIP2 | HIV-1 Tat interactive protein |
| | | | | | | 2, 30 kD |
| 107 | 203290_at | 107 | Hs.198253 | 2.81 | HLA-DQA1 | major histocompatibility |
| | | | | | | complex, class II, DQ alpha 1 |
| 108 | 215275_at | 108 | n/a | 2.10 | | unknown |
| 109 | 221060_s_at | 109 | Hs.159239 | 1.60 | TLR4 | toll-like receptor 4 |
| 110 | 212573_at | 110 | Hs.167115 | 1.44 | KIAA0830 | KIAA0830 protein |
| 111 | 213193_x_at | 111 | Hs.303157 | 1.89 | TRB@ | T cell receptor beta locus |
| 112 | 205568_at | 112 | Hs.104624 | 3.54 | AQP9 | aquaporin 9 |
| 113 | 209281_s_at | 113 | Hs.78546 | 1.65 | ATP2B1 | ATPase, Ca++ transporting, |
| | | | | | | plasma membrane 1 |
| 114 | 204912_at | 114 | Hs.327 | 2.17 | IL10RA | interleukin 10 receptor, alpha |
| 115 | 219099_at | 115 | Hs.24792 | 1.39 | C12orf5 | chromosome 12 open |
| | | | | | | reading frame 5 |
| 116 | 211796_s_at | 116 | Hs.303157 | 2.06 | TRB@ | T cell receptor beta locus |
| 117 | 221724_s_at | 117 | Hs.115515 | 1.84 | CLECSF6 | C-type (calcium dependent, |
| | | | | | | carbohydrate-recognition |
| | | | | | | domain) lectin, superfamily |
| | | | | | | member 6 |
| 118 | 219607_s_at | 118 | Hs.325960 | 1.56 | MS4A4A | membrane-spanning 4- |
| | | | | | | domains, subfamily A, |
| | | | | | | member 4 |
| 119 | 218802_at | 119 | Hs.234149 | 1.91 | FLJ20647 | hypothetical protein |
| | | | | | | FLJ20647 |
| 120 | 221671_x_at | 120 | Hs.156110 | 2.19 | IGKC | immunoglobulin kappa |
| | | | | | | constant |
| 121 | 215121_x_at | 121 | Hs.8997 | 2.56 | HSPA1A, | heat shock 70 kD protein 1A, |
| | | | | | IGL@ | immunoglobulin lambda locus |
| 122 | 202147_s_at | 122 | Hs.7879 | 1.96 | IFRD1 | linterferon-related |
| | | | | | | developmental regulator 1 |
| 123 | 201739_at | 123 | Hs.296323 | 3.73 | SGK | serum/glucocorticoid |
| | | | | | | regulated kinase |
| 124 | 208014_x_at | 124 | Hs.129735 | 1.65 | AD7C-NTP | neuronal thread protein |
| 125 | 211339_s_at | 125 | Hs.211576 | 2.14 | ITK | IL2-inducible T-cell kinase |
| 126 | 211649_x_at | 126 | n/a | 1.84 | | unknown |
| 127 | 202643_s_at | 127 | Hs.211600 | 1.32 | TNFAIP3 | tumor necrosis factor, alpha- |
| | | | | | | induced protein 3 |
| 128 | 218829_s_at | 128 | n/a | 1.95 | | unknown |
| 129 | 204072_s_at | 129 | Hs.181304 | 1.33 | 13CDNA73 | hypothetical protein CG003 |
| 130 | 211824_x_at | 130 | Hs.104305 | 1.38 | DEFCAP | death effector filament- |
| | | | | | | forming Ced-4-like apoptosis |
| | | | | | | protein |
| 131 | 209824_s_at | 131 | Hs.74515 | 2.15 | ARNTL | aryl hydrocarbon receptor |
| | | | | | | nuclear translocator-like |
| 132 | 213539_at | 132 | Hs.95327 | 1.81 | CD3D | CD3D antigen, delta |
| | | | | | | polypeptide (TiT3 complex) |
| 133 | 217143_s_at | 133 | Hs.2014 | 2.01 | TRD@ | T cell receptor delta locus |
| 134 | 204479_at | 134 | Hs.95821 | 1.39 | OSTF1 | osteoclast stimulating factor 1 |
| 135 | 200628_s_at | 135 | Hs.374466 | 1.49 | WARS | tryptophanyl-tRNA |
| | | | | | | synthetase |
| 136 | 201694_s_at | 136 | Hs.326035 | 2.77 | EGR1 | early growth response 1 |
| 137 | 205821_at | 137 | Hs.74085 | 1.51 | D12S2489E | DNA segment on |
| | | | | | | chromosome 12 (unique) |
| | | | | | | 2489 expressed sequence |
| 138 | 209138_x_at | 138 | Hs.181125 | 1.85 | IGLJ3 | immunoglobulin lambda |
| | | | | | | joining 3 |
| 139 | 215242_at | 139 | Hs.97375 | 1.40 | | unknown |
| 140 | 211656_x_at | 140 | Hs.73931 | 1.87 | HLA-DQB1 | major histocompatibility |
| | | | | | | complex, class II, DQ beta 1 |
| 141 | 222221_x_at | 141 | Hs.155119 | 1.45 | EHD1 | EH-domain containing 1 |
| 142 | 208488_s_at | 142 | Hs.193716 | 1.70 | CR1 | complement component |
| | | | | | | (3b/4b) receptor 1, including |
| | | | | | | Knops blood group system |
| 143 | 202437_s_at | 143 | Hs.154654 | 1.66 | CYP1B1 | cytochrome P450, subfamily I |
| | | | | | | (dioxin-inducible), |
| | | | | | | polypeptide 1 (glaucoma 3, |
| | | | | | | primary infantile) |
| 144 | 212286_at | 144 | Hs.27973 | 1.45 | KIAA0874 | KIAA0874 protein |
| 145 | 204959_at | 145 | Hs.153837 | 1.24 | MNDA | myeloid cell nuclear |
| | | | | | | differentiation antigen |
| 146 | 221651_x_at | 146 | Hs.156110 | 2.15 | IGKC | immunoglobulin kappa |
| | | | | | | constant |
| 147 | 201236_s_at | 147 | Hs.75462 | 1.81 | BTG2 | BTG family, member 2 |
| 148 | 211005_at | 148 | Hs.83496 | 1.52 | LAT | linker for activation of T cells |
| 149 | 208078_s_at | 149 | Hs.232068 | 2.27 | TCF8 | transcription factor 8 |
| | | | | | | (represses interleukin 2 |
| | | | | | | expression) |
| 150 | 210018_x_at | 150 | Hs.180566 | 1.61 | MALT1 | mucosa associated lymphoid |
| | | | | | | tissue lymphoma |
| | | | | | | translocation gene 1 |
| 151 | 209273_s_at | 151 | Hs.177776 | 1.56 | MGC4276 | hypothetical protein |
| | | | | | | MGC4276 similar to CG8198 |
| 152 | 213624_at | 152 | Hs.42945 | 1.84 | ASM3A | acid sphingomyelinase-like |
| | | | | | | phosphodiesterase |
| 153 | 208075_s_at | 153 | Hs.251526 | 1.77 | SCYA7 | small inducible cytokine A7 |
| | | | | | | (monocyte chemotactic |
| | | | | | | protein 3) |
| 154 | 212154_at | 154 | Hs.1501 | 1.90 | SDC2 | syndecan 2 (heparan sulfate |
| | | | | | | proteoglycan 1, cell surface- |
| | | | | | | associated, fibroglycan) |

TABLE 3
Top 50 transcripts significantly elevated (p < 0.05)
at baseline in non-responder patient PBMCs

| Affymetrix | SEQ | | | | Fold Diff | p-value |
|---|---|---|---|---|---|---|
| ID | ID NO: | Name | Cyto Band | Unigene ID | (NR/R) | (unequal) |
| 209392_at | 15 | ectonucleotide | 8q24.1 | Hs.174185 | 2.64 | 4.91E−02 |
| | | pyrophosphatase/phosphodiesterase | | | | |
| | | 2 (autotaxin) | | | | |
| 220974_x_at | 25 | similar to rat tricarboxylate | 10q24.31 | Hs.283844 | 2.10 | 1.71E−02 |
| | | carrier-like protein | | | | |
| 206480_at | 66 | leukotriene C4 synthase | 5q35 | Hs.456 | 2.05 | 4.90E−02 |
| 208581_x_at | 1 | metallothionein 1L, | 16q13 | Hs.278462 | 2.04 | 3.13E−02 |
| | | metallothionein 1X | | | | |
| 217165_x_at | 28 | unknown | n/a | n/a | 2.02 | 3.54E−02 |
| 220668_s_at | 39 | DNA (cytosine-5-)- | 20q11.2 | Hs.251673 | 2.00 | 4.00E−02 |
| | | methyltransferase 3 beta | | | | |
| 212185_x_at | 22 | metallothionein 2A | 16q13 | Hs.118786 | 1.89 | 2.55E−02 |
| 209407_s_at | 4 | deformed epidermal | 11p15.5 | Hs.6574 | 1.88 | 2.01E−02 |
| | | autoregulatory factor 1 | | | | |
| | | (Drosophila) | | | | |
| 37384_at | 819 | KIAA0015 gene product | 22q11.22 | Hs.278441 | 1.87 | 4.11E−02 |
| 203725_at | 5 | growth arrest and DNA- | 1p31.2-p31.1 | Hs.80409 | 1.84 | 4.70E−02 |
| | | damage-inducible, alpha | | | | |
| 202942_at | 34 | electron-transfer-flavoprotein, | 19q13.3 | Hs.74047 | 1.78 | 4.69E−02 |
| | | beta polypeptide | | | | |
| 216336_x_at | 3 | unknown | n/a | n/a | 1.73 | 4.92E−02 |
| 212235_at | 592 | KIAA0620 protein | 3q22.1 | Hs.301685 | 1.69 | 4.00E−02 |
| 203089_s_at | 284 | protease, serine, 25 | 2p12 | Hs.115721 | 1.67 | 2.23E−02 |
| 221504_s_at | 77 | ATPase, H+ transporting, | 8p22-q22.3 | Hs.19575 | 1.60 | 4.82E−02 |
| | | lysosomal 50/57 kD V1 subunit H | | | | |
| 220942_x_at | 790 | hypothetical protein, estradiol- | 3q21.1 | Hs.5243 | 1.57 | 2.85E−02 |
| | | induced | | | | |
| 214281_s_at | 23 | zinc finger protein 363 | 4q21.1 | Hs.48297 | 1.56 | 2.43E−02 |
| 203091_at | 285 | far upstream element (FUSE) | 1p31.1 | Hs.118962 | 1.56 | 3.28E−02 |
| | | binding protein 1 | | | | |
| 204050_s_at | 50 | clathrin, light polypeptide (Lca) | 9p13 | Hs.104143 | 1.54 | 4.99E−02 |
| 210093_s_at | 14 | mago-nashi homolog, | 1p34-p33 | Hs.57904 | 1.52 | 2.43E−04 |
| | | proliferation-associated | | | | |
| | | (Drosophila) | | | | |
| 217226_s_at | 689 | paired mesoderm homeo box | 10q24.31, | Hs.155606 | 1.52 | 8.44E−03 |
| | | 1, similar to rat tricarboxylate | 1q24 | | | |
| | | carrier-like protein | | | | |
| 218807_at | 26 | vav 3 oncogene | 1p13.2 | Hs.267659 | 1.52 | 2.11E−02 |
| 200824_at | 172 | glutathione S-transferase pi | 11q13 | Hs.226795 | 1.51 | 2.96E−02 |
| 221923_s_at | 805 | nucleophosmin (nucleolar | 5q35 | Hs.9614 | 1.51 | 3.95E−03 |
| | | phosphoprotein B23, numatrin) | | | | |
| 202854_at | 269 | hypoxanthine | Xq26.1 | Hs.82314 | 1.51 | 1.32E−02 |
| | | phosphoribosyltransferase 1 | | | | |
| | | (Lesch-Nyhan syndrome) | | | | |
| 201241_at | 197 | DEAD/H (Asp-Glu-Ala- | 2p24 | Hs.78580 | 1.51 | 3.98E−02 |
| | | Asp/His) box polypeptide 1 | | | | |
| 203720_s_at | 305 | excision repair cross- | 19q13.2-q13.3 | Hs.59544 | 1.49 | 2.55E−02 |
| | | complementing rodent repair | | | | |
| | | deficiency, complementation | | | | |
| | | group 1 (includes overlapping | | | | |
| | | antisense sequence) | | | | |
| 211941_s_at | 578 | prostatic binding protein | 12q24.22 | Hs.80423 | 1.48 | 5.88E−03 |
| 218049_s_at | 31 | mitochondrial ribosomal | 8q22.1-q22.3 | Hs.333823 | 1.48 | 4.24E−02 |
| | | protein L13 | | | | |
| 218795_at | 737 | LPAP for lysophosphatidic | 1q21 | Hs.15871 | 1.48 | 4.03E−02 |
| | | acid phosphatase | | | | |
| 212749_s_at | 606 | zinc finger protein 363 | 4q21.1 | Hs.48297 | 1.47 | 2.06E−02 |
| 200960_x_at | 179 | clathrin, light polypeptide (Lca) | 9p13 | Hs.104143 | 1.46 | 4.43E−02 |
| 201577_at | 221 | non-metastatic cells 1, protein | 17q21.3 | Hs.118638 | 1.46 | 3.31E−02 |
| | | (NM23A) expressed in | | | | |
| 205711_x_at | 412 | ATP synthase, H+ | 10q22-q23, | Hs.155433 | 1.44 | 2.59E−02 |
| | | transporting, mitochondrial F1 | 8p22-p21.3 | | | |
| | | complex, gamma polypeptide | | | | |
| | | 1, CCR4-NOT transcription | | | | |
| | | complex, subunit 7 | | | | |
| 213366_x_at | 625 | ATP synthase, H+ | 10q22-q23, | Hs.155433 | 1.44 | 4.59E−02 |
| | | transporting, mitochondrial F1 | 8p22-p21.3 | | | |
| | | complex, gamma polypeptide | | | | |
| | | 1, CCR4-NOT transcription | | | | |
| | | complex, subunit 7 | | | | |
| 217942_at | 702 | mitochondrial ribosomal | 12p11 | Hs.10724 | 1.44 | 3.24E−02 |
| | | protein S35 | | | | |
| 208713_at | 468 | E1B-55 kDa-associated protein 5 | 19q13.31 | Hs.155218 | 1.44 | 1.66E−02 |
| 201765_s_at | 225 | hexosaminidase A (alpha | 15q23-q24 | Hs.119403 | 1.43 | 4.74E−02 |
| | | polypeptide) | | | | |
| 216295_s_at | 679 | clathrin, light polypeptide (Lca) | 9p13 | Hs.348345 | 1.43 | 4.32E−02 |
| 202929_s_at | 275 | D-dopachrome tautomerase | 22q11.23 | Hs.180015 | 1.43 | 4.87E−02 |
| 217871_s_at | 700 | macrophage migration | 22q11.23 | Hs.73798 | 1.43 | 3.36E−02 |
| | | inhibitory factor (glycosylation- | | | | |
| | | inhibiting factor) | | | | |
| 218078_s_at | 711 | zinc finger, DHHC domain | 3p21.32 | Hs.14896 | 1.42 | 1.63E−02 |
| | | containing 3 | | | | |
| 208870_x_at | 474 | ATP synthase, H+ | 10q22-q23, | Hs.155433 | 1.42 | 1.95E−02 |
| | | transporting, mitochondrial F1 | 8p22-p21.3 | | | |
| | | complex, gamma polypeptide | | | | |
| | | 1, CCR4-NOT transcription | | | | |
| | | complex, subunit 7 | | | | |
| 200822_x_at | 171 | triosephosphate isomerase 1 | 12p13 | Hs.83848 | 1.42 | 4.53E−02 |
| 203103_s_at | 286 | nuclear matrix protein | 11q12.2 | Hs.173980 | 1.41 | 3.70E−02 |
| | | NMP200 related to splicing | | | | |
| | | factor PRP19 | | | | |
| 213507_s_at | 628 | karyopherin (importin) beta 1 | 17q21 | Hs.180446 | 1.41 | 1.07E−02 |
| 201231_s_at | 195 | enolase 1, (alpha) | 1p36.3-p36.2 | Hs.254105 | 1.40 | 2.89E−02 |
| 204905_s_at | 376 | eukaryotic translation | 6p24.3-p25.1 | Hs.298581 | 1.39 | 3.32E−02 |
| | | elongation factor 1 epsilon 1 | | | | |
| 203177_x_at | 19 | transcription factor A, | 10q21 | Hs.75133 | 1.39 | 2.82E−02 |
| | | mitochondrial | | | | |
| 218154_at | 714 | hypothetical protein FLJ12150 | 8q24.3 | Hs.118983 | 1.39 | 4.30E−02 |

TABLE 4
Top 50 transcripts significantly elevated (p < 0.05) at
baseline in responder patient PBMCs

| Affymetrix | SEQ ID | | | | Fold Diff | p-value |
|---|---|---|---|---|---|---|
| ID | NO: | Name | Cyto Band | Unigene ID | (R/NR) | (unequal) |
| 218559_s_at | 727 | v-maf musculoaponeurotic | 20q11.2-q13.1 | Hs.169487 | 7.33 | 1.30E−02 |
| | | fibrosarcoma oncogene | | | | |
| | | homolog B (avian) | | | | |
| 209728_at | 509 | major histocompatibility | 6p21.3 | Hs.318720 | 6.49 | 5.81E−03 |
| | | complex, class II, DR beta 4 | | | | |
| 204614_at | 356 | serine (or cysteine) proteinase | 18q21.3 | Hs.75716 | 4.11 | 4.20E−02 |
| | | inhibitor, clade B (ovalbumin), | | | | |
| | | member 2 | | | | |
| 209671_x_at | 85 | T cell receptor alpha locus | 14q11.2 | Hs.74647 | 3.95 | 8.98E−03 |
| 210972_x_at | 81 | T cell receptor alpha locus | 14q11.2 | Hs.74647 | 3.89 | 6.39E−03 |
| 201739_at | 123 | serum/glucocorticoid | 6q23 | Hs.296323 | 3.73 | 5.87E−04 |
| | | regulated kinase | | | | |
| 219593_at | 79 | peptide transporter 3 | 11q13.1 | Hs.237856 | 3.57 | 7.04E−04 |
| 205568_at | 112 | aquaporin 9 | 15q22.1-22.2 | Hs.104624 | 3.54 | 8.87E−04 |
| 204885_s_at | 372 | mesothelin | 16p13.12 | Hs.155981 | 3.54 | 2.13E−02 |
| 211571_s_at | 564 | chondroitin sulfate | 5q14.3 | Hs.81800 | 3.45 | 4.23E−02 |
| | | proteoglycan 2 (versican) | | | | |
| 210655_s_at | 545 | forkhead box O3A | 6q21 | Hs.14845 | 3.36 | 5.20E−03 |
| 213338_at | 622 | Ras-induced senescence 1 | 3p21.3 | Hs.35861 | 3.29 | 1.67E−02 |
| 213524_s_at | 630 | putative lymphocyte G0/G1 | 1q32.2-q41 | Hs.95910 | 3.28 | 1.78E−03 |
| | | switch gene | | | | |
| 221602_s_at | 798 | regulator of Fas-induced | 1q31.3 | Hs.58831 | 3.19 | 8.83E−03 |
| | | apoptosis | | | | |
| 205220_at | 82 | putative chemokine receptor; | 12q24.31 | Hs.137555 | 3.11 | 7.86E−04 |
| | | GTP-binding protein | | | | |
| 208450_at | 461 | lectin, galactoside-binding, | 22q13.1 | Hs.113987 | 2.99 | 3.18E−02 |
| | | soluble, 2 (galectin 2) | | | | |
| 205898_at | 416 | chemokine (C—X3—C) | 3p21.3 | Hs.78913 | 2.98 | 2.29E−02 |
| | | receptor 1 | | | | |
| 212099_at | 584 | ras homolog gene family, | 2pter-p12 | Hs.204354 | 2.96 | 3.05E−03 |
| | | member B | | | | |
| 218856_at | 742 | hypothetical protein | 6p12.3, 6p21.1-12.2 | Hs.65403 | 2.90 | 8.84E−03 |
| | | LOC51323, tumor necrosis | | | | |
| | | factor receptor superfamily, | | | | |
| | | member 21 | | | | |
| 220088_at | 775 | complement component 5 | 19q13.3-q13.4 | Hs.2161 | 2.86 | 6.44E−03 |
| | | receptor 1 (C5a ligand) | | | | |
| 221698_s_at | 799 | C-type (calcium dependent, | 12p13.2-p12.3 | Hs.161786 | 2.83 | 1.85E−03 |
| | | carbohydrate-recognition | | | | |
| | | domain) lectin, superfamily | | | | |
| | | member 12 | | | | |
| 201743_at | 224 | CD14 antigen | 5q31.1 | Hs.75627 | 2.83 | 2.71E−02 |
| 212657_s_at | 604 | interleukin 1 receptor | 2q14.2 | Hs.81134 | 2.83 | 4.41E−03 |
| | | antagonist | | | | |
| 203290_at | 107 | major histocompatibility | 6p21.3 | Hs.198253 | 2.81 | 2.06E−02 |
| | | complex, class II, DQ alpha 1 | | | | |
| 204588_s_at | 354 | solute carrier family 7 (cationic | 14q11.2 | Hs.194693 | 2.81 | 3.88E−03 |
| | | amino acid transporter, y+ | | | | |
| | | system), member 7 | | | | |
| 211506_s_at | 561 | interleukin 8 | 4q13-q21 | Hs.624 | 2.80 | 1.47E−03 |
| 201694_s_at | 136 | early growth response 1 | 5q31.1 | Hs.326035 | 2.77 | 1.04E−03 |
| 204890_s_at | 373 | lymphocyte-specific protein | 1p34.3 | Hs.1765 | 2.64 | 2.12E−02 |
| | | tyrosine kinase | | | | |
| 221558_s_at | 88 | lymphoid enhancer-binding | 4q23-q25 | Hs.44865 | 2.63 | 1.82E−02 |
| | | factor 1 | | | | |
| 203907_s_at | 90 | KIAA0763 gene product | 3p25.1 | Hs.4764 | 2.63 | 1.45E−03 |
| 203066_at | 282 | B cell RAG associated protein | 10q26 | Hs.6079 | 2.61 | 1.90E−03 |
| 219434_at | 87 | triggering receptor expressed | 6p21.1 | Hs.283022 | 2.61 | 2.06E−02 |
| | | on myeloid cells 1 | | | | |
| 216191_s_at | 677 | T cell receptor delta locus | 14q11.2 | Hs.2014 | 2.59 | 1.80E−02 |
| 205114_s_at | 382 | small inducible cytokine A3 | 17q11-q21 | Hs.73817 | 2.57 | 3.76E−02 |
| 215223_s_at | 668 | superoxide dismutase 2, | 6q25.3 | Hs.372783 | 2.57 | 1.30E−03 |
| | | mitochondrial | | | | |
| 216491_x_at | 682 | unknown | n/a | n/a | 2.55 | 4.12E−02 |
| 217739_s_at | 695 | pre-B-cell colony-enhancing | 7q11.23 | Hs.239138 | 2.53 | 1.04E−03 |
| | | factor | | | | |
| 201631_s_at | 223 | immediate early response 3 | 6p21.3 | Hs.76095 | 2.47 | 2.21E−02 |
| 202086_at | 238 | myxovirus (influenza virus) | 21q22.3 | Hs.76391 | 2.47 | 1.04E−03 |
| | | resistance 1, interferon- | | | | |
| | | inducible protein p78 (mouse) | | | | |
| 204141_at | 331 | tubulin, beta polypeptide | 6p21.3 | Hs.336780 | 2.46 | 3.35E−02 |
| 209670_at | 507 | T cell receptor alpha locus | 14q11.2 | Hs.74647 | 2.46 | 3.71E−02 |
| 219528_s_at | 762 | B-cell CLL/lymphoma 11B | 14q32.31-q32.32 | Hs.57987 | 2.45 | 3.11E−02 |
| | | (zinc finger protein) | | | | |
| 206150_at | 426 | tumor necrosis factor receptor | 12p13 | Hs.180841 | 2.44 | 1.94E−02 |
| | | superfamily, member 7 | | | | |
| 201506_at | 213 | transforming growth factor, | 5q31 | Hs.118787 | 2.42 | 4.20E−02 |
| | | beta-induced, 68 kD | | | | |
| 203939_at | 314 | 5′-nucleotidase, ecto (CD73) | 6q14-q21 | Hs.153952 | 2.42 | 1.91E−02 |
| 205419_at | 396 | Epstein-Barr virus induced | 13q32.3 | Hs.784 | 2.39 | 1.56E−03 |
| | | gene 2 (lymphocyte-specific G | | | | |
| | | protein-coupled receptor) | | | | |
| 212812_at | 98 | unknown | n/a | Hs.288232 | 2.39 | 1.11E−04 |
| 217378_x_at | 692 | unknown | n/a | n/a | 2.38 | 2.11E−02 |
| 211135_x_at | 555 | leukocyte immunoglobulin-like | 19q13.4 | Hs.105928 | 2.37 | 1.57E−02 |
| | | receptor, subfamily B (with TM | | | | |
| | | and ITIM domains), member 3 | | | | |
| 204006_s_at | 318 | Fc fragment of IgG, low affinity | 1q23 | Hs.372679 | 2.36 | 4.30E−02 |
| | | IIIa, receptor for (CD16), Fc | | | | |
| | | fragment of IgG, low affinity | | | | |
| | | IIIb, receptor for (CD16) | | | | |

Genes Associated with the Onset of Veno-Occlusive Disease

Veno-occlusive disease (VOD) is one of the most serious complications following hematopoietic stem cell transplantation and is associated with a very high mortality in its severe form. Comparison of pretreatment PBMC profiles from the leukemia patients who experienced VOD with the PBMC profiles from the patients who did not experience VOD identifies significant transcripts that appear to be correlated with this serious adverse event prior to therapy.

To identify transcripts with significant differences in expression at baseline between the patients who experienced VOD and the non-VOD patients, average fold differences between VOD and non-VOD patient profiles were calculated by dividing the mean level of expression in the baseline VOD profiles by the mean level of expression in the baseline non-VOD profiles. A Student's t-test (two-sample, unequal variance) was used to assess the significance of the difference in expression between the groups.

Genes whose expression levels are significantly elevated (p<0.05) at baseline in VOD patients are shown in Table 5. Genes whose expression levels are significantly repressed (p<0.05) at baseline in VOD patients are shown in Table 6. Of interest, P-selectin ligand was one of the transcripts most significantly elevated at baseline in patients who experienced VOD. Without wishing to be bound by theory, the elevation in this transcript may be a biomarker indicative of endothelial damage which has been suggested to play a role in transplant-associated diseases such as graft-versus-host disease, sepsis, and VOD.

TABLE 5
Top 50 Transcripts significantly elevated (p < 0.05)
at baseline in VOD patient PBMCs

| | SEQ ID | | | | Fold Diff | p-value |
|---|---|---|---|---|---|---|
| Affymetrix ID | NO:: | Name | Cyto Band | Unigene ID | (VOD/non-VOD) | (unequal) |
| 204020_at | 321 | purine-rich element binding protein A | 5q31 | Hs.29117 | 2.096551724 | 0.025737029 |
| 202742_s_at | 264 | protein kinase, cAMP-dependent, | 1p36.1 | Hs.87773 | 2.031746032 | 0.023084697 |
| | | catalytic, beta | | | | |
| 209879_at | 516 | selectin P ligand | 12q24 | Hs.79283 | 2.02247191 | 0.024750558 |
| AFFX-r2- | 826 | n/a | n/a | n/a | 1.967450271 | 0.00094123 |
| Hs28SrRNA-3_at | | | | | | |
| 217986_s_at | 704 | bromodomain adjacent to zinc finger | 14q12-q13 | Hs.8858 | 1.948186528 | 0.040961702 |
| | | domain, 1A | | | | |
| 202322_s_at | 247 | geranylgeranyl diphosphate | 1q43 | Hs.55498 | 1.806451613 | 0.008621905 |
| | | synthase 1 | | | | |
| AFFX- | 825 | n/a | n/a | n/a | 1.789173789 | 0.007668769 |
| M27830_5_at | | | | | | |
| 219974_x_at | 772 | uncharacterized hypothalamus | 6q23.1 | Hs.239218 | 1.741496599 | 0.026918594 |
| | | protein HCDASE | | | | |
| 201964_at | 231 | KIAA0625 protein | 9q34.3 | Hs.154919 | 1.739130435 | 0.025540988 |
| 202741_at | 263 | n/a | 1p36.1 | Hs.417060 | 1.737931034 | 0.003565502 |
| 203947_at | 315 | cleavage stimulation factor, 3′ pre- | 11p12 | Hs.180034 | 1.723076923 | 0.011499059 |
| | | RNA, subunit 3, 77 kDa | | | | |
| 218642_s_at | 729 | hypothetical protein MGC2217 | 8q11.22 | Hs.323164 | 1.686486486 | 0.010323657 |
| 200860_s_at | 173 | KIAA1007 protein | 16q21 | Hs.279949 | 1.682403433 | 0.018297378 |
| 201027_s_at | 185 | translation initiation factor IF2 | 2p11.1-q11.1 | Hs.158688 | 1.680672269 | 0.032120458 |
| 213361_at | 624 | tudor repeat associator with | 9q22.33 | Hs.283761 | 1.656804734 | 0.027072176 |
| | | PCTAIRE 2 | | | | |
| 220956_s_at | 791 | egl nine homolog 2 (C. elegans) | 19q13.2 | Hs.324277 | 1.653631285 | 0.007996997 |
| 218646_at | 730 | hypothetical protein FLJ20534 | 4q32.3 | Hs.44344 | 1.619047619 | 0.019526095 |
| 200604_s_at | 156 | protein kinase, cAMP-dependent, | 17q23-q24 | Hs.183037 | 1.608938547 | 0.040659084 |
| | | regulatory, type I, alpha (tissue | | | | |
| | | specific extinguisher 1) | | | | |
| 201989_s_at | 233 | cAMP responsive element binding | 12p13 | Hs.13313 | 1.608247423 | 0.042105857 |
| | | protein-like 2 | | | | |
| 217993_s_at | 706 | methionine adenosyltransferase II, | 5q34-q35.1 | Hs.54642 | 1.597964377 | 0.002167131 |
| | | beta | | | | |
| 204613_at | 355 | phospholipase C, gamma 2 | 16q24.1 | Hs.75648 | 1.592039801 | 0.012601371 |
| | | (phosphatidylinositol-specific) | | | | |
| 201142_at | 191 | eukaryotic translation initiation factor | 14q23.3 | Hs.151777 | 1.567010309 | 1.80074E−06 |
| | | 2, subunit 1 alpha, 35 kDa | | | | |
| 219649_at | 765 | dolichyl-P-Glc: Man9GlcNAc2-PP- | 1p31.3 | Hs.80042 | 1.565217391 | 0.021274365 |
| | | dolichylglucosyltransferase | | | | |
| 209907_s_at | 519 | intersectin 2 | 2pter-p25.1 | Hs.166184 | 1.5625 | 0.02410118 |
| 210502_s_at | 540 | peptidylprolyl isomerase E | 1p32 | Hs.379815 | 1.555555556 | 0.000233425 |
| | | (cyclophilin E) | | | | |
| 209903_s_at | 517 | ataxia telangiectasia and Rad3 | 3q22-q24 | Hs.77613 | 1.551515152 | 0.016402019 |
| | | related | | | | |
| 212402_at | 598 | KIAA0853 protein | 13q14.11 | Hs.136102 | 1.543147208 | 1.96044E−06 |
| 202003_s_at | 234 | acetyl-Coenzyme A acyltransferase | 18q21.1 | Hs.356176 | 1.538461538 | 0.031540874 |
| | | 2 (mitochondrial 3-oxoacyl- | | | | |
| | | Coenzyme A thiolase) | | | | |
| 220933_s_at | 789 | hypothetical protein FLJ13409 | 9q21 | Hs.30732 | 1.536723164 | 0.030072848 |
| 208911_s_at | 479 | pyruvate dehydrogenase (lipoamide) | 3p21.1-p14.2 | Hs.979 | 1.531914894 | 0.020768712 |
| | | beta | | | | |
| 212697_at | 605 | n/a | n/a | Hs.432850 | 1.519832985 | 0.022783857 |
| 219940_s_at | 770 | hypothetical protein FLJ11305 | 13q34 | Hs.7049 | 1.514403292 | 0.001555339 |
| 212754_s_at | 607 | KIAA1040 protein | 12q13.13 | Hs.9846 | 1.505882353 | 0.037849628 |
| 207614_s_at | 453 | cullin 1 | 7q34-q35 | Hs.14541 | 1.496402878 | 0.049509373 |
| 209096_at | 483 | ubiquitin-conjugating enzyme E2 | 8q11.1 | Hs.79300 | 1.493975904 | 0.047033925 |
| | | variant 2 | | | | |
| 200802_at | 167 | seryl-tRNA synthetase | 1p13.3-p13.1 | Hs.144063 | 1.488372093 | 0.005291866 |
| 220408_x_at | 779 | transcription factor (p38 interacting | 13q13.1-q13.2 | Hs.376447 | 1.484848485 | 0.035433399 |
| | | protein) | | | | |
| 204780_s_at | 364 | tumor necrosis factor receptor | 10q24.1 | Hs.426662 | 1.476923077 | 0.000371305 |
| | | superfamily, member 6 | | | | |
| 203879_at | 310 | phosphoinositide-3-kinase, catalytic, | 1p36.2 | Hs.162808 | 1.471406491 | 0.035824787 |
| | | delta polypeptide | | | | |
| 201384_s_at | 204 | membrane component, | 17q21.1 | Hs.277721 | 1.46875 | 0.009771907 |
| | | chromosome 17, surface marker 2 | | | | |
| | | (ovarian carcinoma antigen CA125) | | | | |
| 212588_at | 603 | protein tyrosine phosphatase, | 1q31-q32 | Hs.170121 | 1.461700632 | 0.048016891 |
| | | receptor type, C | | | | |
| 219033_at | 751 | hypothetical protein FLJ21308 | 5q11.1 | Hs.406232 | 1.459016393 | 0.02208168 |
| 203073_at | 283 | component of oligomeric golgi | 1q42.13 | Hs.82399 | 1.457489879 | 0.008447959 |
| | | complex 2 | | | | |
| 206332_s_at | 430 | interferon, gamma-inducible protein | 1q22 | Hs.155530 | 1.455696203 | 0.027832428 |
| | | 16 | | | | |
| 202868_s_at | 272 | POP4 (processing of precursor, | 19q13.11 | Hs.82238 | 1.449275362 | 0.021497345 |
| | | S. cerevisiae) homolog | | | | |
| 218249_at | 718 | zinc finger, DHHC domain | 10q26.11 | Hs.22353 | 1.427509294 | 0.001378715 |
| | | containing 6 | | | | |
| 212530_at | 602 | NIMA (never in mitosis gene a)- | 1q31.3 | Hs.24119 | 1.418719212 | 0.035013309 |
| | | related kinase 7 | | | | |
| 218463_s_at | 725 | MUS81 endonuclease | 11q13 | Hs.288798 | 1.403508772 | 0.034273747 |
| 213115_at | 613 | n/a | n/a | n/a | 1.398907104 | 0.038806001 |
| 218103_at | 712 | FtsJ homolog 3 (E. coli) | 17q23 | Hs.257486 | 1.393258427 | 5.58595E−05 |

TABLE 6
Top 50 transcripts significantly repressed (p < 0.05)
at baseline in VOD patient PBMCs

| | | | | | Fold Diff | p-value |
|---|---|---|---|---|---|---|
| Affymetrix ID | SEQ ID NO: | Name | Cyto Band | Unigene ID | (VOD/non-VOD) | (unequal) |
| 217023_x_at | 688 | tryptase beta 1, tryptase beta 2 | 16p13.3 | Hs.294158, Hs.405479 | 0.131687243 | 0.000341 |
| 210084_x_at | 525 | tryptase beta 2, tryptase, alpha | 16p13.3 | Hs.294158 | 0.133828996 | 0.000347153 |
| 208029_s_at | 58 | lysosomal associated protein | 8q22.1 | Hs.296398 | 0.133891213 | 0.020766934 |
| | | transmembrane 4 beta | | | | |
| 213844_at | 638 | homeo box A5 | 7p15-p14 | Hs.37034 | 0.148514851 | 0.003338613 |
| 215382_x_at | 670 | tryptase, alpha | 16p13.3 | Hs.334455 | 0.155477032 | 0.000156058 |
| 205683_x_at | 411 | tryptase beta 1, tryptase beta 2, tryptase, | 16p13.3 | Hs.405479 | 0.158102767 | 0.00154079 |
| | | alpha | | | | |
| 216474_x_at | 681 | tryptase beta 1, tryptase beta 2, tryptase, | 16p13.3 | Hs.334455 | 0.15954416 | 0.000338402 |
| | | alpha | | | | |
| 208789_at | 470 | polymerase I and transcript release factor | 17q21.2 | Hs.29759 | 0.172972973 | 0.004109481 |
| 202016_at | 235 | mesoderm specific transcript homolog | 7q32 | Hs.79284 | 0.176239182 | 0.001253864 |
| | | (mouse) | | | | |
| 207134_x_at | 447 | tryptase beta 1, tryptase beta 2, tryptase, | 16p13.3 | Hs.294158 | 0.180722892 | 0.002582561 |
| | | alpha | | | | |
| 214039_s_at | 643 | lysosomal associated protein | 8q22.1 | Hs.296398 | 0.221343874 | 0.015962264 |
| | | transmembrane 4 beta | | | | |
| 201015_s_at | 184 | junction plakoglobin | 17q21 | Hs.2340 | 0.227642276 | 2.96697E−06 |
| 202112_at | 240 | von Willebrand factor | 12p13.3 | Hs.110802 | 0.231884058 | 0.000771533 |
| 36711_at | 817 | v-maf musculoaponeurotic fibrosarcoma | 22q13.1 | Hs.51305 | 0.243093923 | 0.000110895 |
| | | oncogene homolog F (avian) | | | | |
| 207741_x_at | 456 | tryptase, alpha | 16p13.3 | Hs.334455 | 0.244741874 | 0.000539503 |
| 209395_at | 495 | chitinase 3-like 1 (cartilage glycoprotein- | 1q31.1 | Hs.75184 | 0.266666667 | 0.006968551 |
| | | 39) | | | | |
| 205131_x_at | 383 | stem cell growth factor; lymphocyte | 19q13.3 | Hs.425339 | 0.266666667 | 0.01030592 |
| | | secreted C-type lectin | | | | |
| 201005_at | 183 | CD9 antigen (p24) | 12p13.3 | Hs.1244 | 0.270613108 | 0.001191345 |
| 215111_s_at | 666 | transforming growth factor beta-stimulated | 13q14 | Hs.114360 | 0.279957582 | 0.00118603 |
| | | protein TSC-22 | | | | |
| 205624_at | 409 | carboxypeptidase A3 (mast cell) | 3q21-q25 | Hs.646 | 0.282225237 | 0.00249997 |
| 206067_s_at | 423 | Wilms tumor 1 | 11p13 | Hs.1145 | 0.282352941 | 0.001463202 |
| 201596_x_at | 222 | glutamate receptor, ionotropic, N-methyl D- | 12q13 | Hs.406013 | 0.292358804 | 0.002605841 |
| | | asparate-associated protein 1 (glutamate | | | | |
| | | binding), keratin 18 | | | | |
| 213479_at | 627 | neuronal pentraxin II | 7q21.3-q22.1 | Hs.3281 | 0.298507463 | 0.046185388 |
| 201324_at | 201 | epithelial membrane protein 1 | 12p12.3 | Hs.79368 | 0.299065421 | 0.001554754 |
| 210783_x_at | 549 | stem cell growth factor; lymphocyte | 19q13.3 | Hs.425339 | 0.301886792 | 0.009424594 |
| | | secreted C-type lectin | | | | |
| 216202_s_at | 678 | serine palmitoyltransferase, long chain | 14q24.3-q31 | Hs.59403 | 0.306220096 | 0.000219065 |
| | | base subunit 2 | | | | |
| 218880_at | 744 | FOS-like antigen 2 | 2p23-p22 | Hs.301612 | 0.310679612 | 0.000328157 |
| 206461_x_at | 435 | metallothionein 1H | 16q13 | Hs.2667 | 0.310679612 | 0.001303906 |
| 204885_s_at | 372 | mesothelin | 16p13.12 | Hs.155981 | 0.310679612 | 0.021690405 |
| 220377_at | 778 | chromosome 14 open reading frame 110 | 14q32.33 | Hs.128155 | 0.315789474 | 0.003681392 |
| 204011_at | 319 | sprouty homolog 2 (Drosophila) | 13q22.2 | Hs.18676 | 0.32 | 0.00124785 |
| 211948_x_at | 579 | KIAA1096 protein | 1q23.3 | Hs.69559 | 0.32 | 0.008446106 |
| 208886_at | 476 | H1 histone family, member 0 | 22q13.1 | Hs.226117 | 0.321715818 | 0.00641406 |
| 215047_at | 665 | BIA2 | 1q44 | Hs.51692 | 0.322147651 | 0.022774503 |
| 209905_at | 518 | homeo box A9 | 7p15-p14 | Hs.127428 | 0.322496749 | 0.022921003 |
| 218332_at | 721 | brain expressed, X-linked 1 | Xq21-q23 | Hs.334370 | 0.325 | 0.026696331 |
| 203411_s_at | 293 | lamin A/C | 1q21.2-q21.3 | Hs.377973 | 0.329411765 | 0.000122251 |
| 209774_x_at | 511 | chemokine (C—X—C motif) ligand 1 | 4q21 | Hs.75765 | 0.33256351 | 0.002389608 |
| | | (melanoma growth stimulating activity, | | | | |
| | | alpha), chemokine (C—X—C motif) | | | | |
| | | ligand 2 | | | | |
| 209757_s_at | 70 | v-myc myelocytomatosis viral related | 2p24.1 | Hs.25960 | 0.333333333 | 0.0002004 |
| | | oncogene, neuroblastoma derived (avian) | | | | |
| 201830_s_at | 227 | neuroepithelial cell transforming gene 1 | 10p15 | Hs.25155 | 0.335078534 | 0.000181408 |
| 219837_s_at | 769 | cytokine-like protein C17 | 4p16-p15 | Hs.13872 | 0.347826087 | 0.009008447 |
| 205051_s_at | 380 | v-kit Hardy-Zuckerman 4 feline sarcoma | 4q11-q12 | Hs.81665 | 0.348993289 | 0.006943974 |
| | | viral oncogene homolog | | | | |
| 211709_s_at | 566 | stem cell growth factor; lymphocyte | 19q13.3 | Hs.425339 | 0.354948805 | 0.033343631 |
| | | secreted C-type lectin | | | | |
| 210665_at | 75 | tissue factor pathway inhibitor (lipoprotein- | 2q31-q32.1 | Hs.170279 | 0.355555556 | 0.001918239 |
| | | associated coagulation inhibitor) | | | | |
| 209301_at | 491 | carbonic anhydrase II | 8q22 | Hs.155097 | 0.355555556 | 0.003901677 |
| 204468_s_at | 9 | tyrosine kinase with immunoglobulin and | 1p34-p33 | Hs.78824 | 0.36036036 | 0.034680165 |
| | | epidermal growth factor homology domains | | | | |
| 208767_s_at | 469 | lysosomal associated protein | 8q22.1 | Hs.296398 | 0.361111111 | 0.022507793 |
| | | transmembrane 4 beta | | | | |
| 209183_s_at | 485 | decidual protein induced by progesterone | 10q11.23 | Hs.93675 | 0.363636364 | 0.0038473 |
| 213260_at | 619 | | | Hs.284186 | 0.366666667 | 0.030189907 |
| 209488_s_at | 497 | RNA-binding protein gene with multiple | 8p12-p11 | Hs.80248 | 0.367816092 | 0.013648398 |
| | | splicing | | | | |

### Identification of Leukemia Diagnostic Genes

The above described methods can also be used to identify leukemia diagnostic genes (also referred to as disease genes). Each of these genes is differentially expressed in PBMCs of leukemia patients relative to PBMCs of leukemia-free or disease-free humans. In many cases, the average PBMC expression level of a leukemia disease gene in leukemia patients is statistically different from that in leukemia-free or disease-free humans. For example, the p-value of a Student's t-test for the observed difference can be no more than 0.05, 0.01, 0.005, 0.001, 0.0005, 0.0001, or less. In many other cases, the difference between the average PBMC expression levels of a leukemia disease gene in leukemia patients and that in leukemia-free humans is at least 2, 3, 4, 5, 10, 20, or more folds. The leukemia disease genes of the present invention can be used to detect the presence or absence, or monitor the development, progression or treatment of leukemia in a human of interest.

Leukemia disease genes can also be identified by correlating PBMC expression profiles with a class distinction under a class-based correlation metric (e.g., the nearest-neighbor analysis or the significance method of microarrays (SAM) method). The class distinction represents an idealized gene expression pattern in PBMCs of leukemia patients and disease-free humans. In many examples, the correlation between the PBMC expression profile of a leukemia disease gene and the class distinction is above the 1%, 5%, 10%, 25%, or 50% significance level under a permutation test. Gene classifiers can be constructed using the leukemia disease genes of the present invention. These classifiers can effectively predict class membership (e.g., leukemia versus leukemia-free) of a human of interest.

### Identification of AML Diagnosis Genes Using HG-U133A Microarrays

As an example, AML-associated expression patterns in peripheral blood were identified by using the U133A gene chip platform. Mean levels of baseline gene expression in PBMCs from a group of disease-free volunteers (n=20) were compared with mean levels of corresponding baseline gene expression in PBMCs from AML patients (n=36). Transcripts showing elevated or decreased levels in PBMCs of AML patients relative to healthy controls were identified. Examples of these transcripts are depicted in Table 7. Each transcript in Table 7 has at least 2-fold difference in the mean level of expression between AML PBMCs and disease-free PBMCs (“AML/Disease-Free”). The p-value of the Student's t-test (unequal variances) for the observed difference (“P-Value”) is also shown in Table 7. “COV” refers to coefficient of variance.

TABLE 7
Example of AML Disease Genes Differentially Expressed in PBMCs of AML Patients Relative to Disease-Free Volunteers

| | | AML/ | | | COV | | | |
|---|---|---|---|---|---|---|---|---|
| | SEQ ID | Disease- | | COV | (Disease | Gene | | Unigene |
| Qualifier | NO: | Free | P-Value | (AML) | Free) | Symbol | Gene Name | No. |
| 203948_s_at | 316 | 46.69 | 4.63E−06 | 108.53% | 33.68% | MPO | myeloperoxidase | Hs.1817 |
| 203949_at | 317 | 35.14 | 1.19E−06 | 99.53% | 29.31% | MPO | myeloperoxidase | Hs.1817 |
| 206310_at | 429 | 22.75 | 3.86E−06 | | | SPINK2 | serine protease inhibitor, Kazal | Hs.98243 |
| | | | | | | | type, 2 (acrosin-trypsin | |
| | | | | | | | inhibitor) | |
| 209905_at | 518 | 21.08 | 5.44E−05 | | | HOXA9 | homeo box A9 | Hs.127428 |
| 214575_s_at | 658 | 20.02 | 3.88E−04 | 145.25% | 28.21% | AZU1 | azurocidin 1 (cationic | Hs.72885 |
| | | | | | | | antimicrobial protein 37) | |
| 206871_at | 444 | 18.41 | 1.23E−04 | 131.40% | 48.57% | ELA2 | elastase 2, neutrophil | Hs.99863 |
| 214651_s_at | 660 | 16.25 | 5.98E−05 | 123.43% | 21.22% | HOXA9 | homeo box A9 | Hs.127428 |
| 205653_at | 410 | 14.76 | 1.24E−03 | 159.20% | 28.58% | CTSG | cathepsin G | Hs.100764 |
| 210084_x_at | 525 | 14.18 | 1.20E−04 | | | | tryptase beta 1, tryptase, alpha | Hs.347933 |
| 205683_x_at | 411 | 13.92 | 4.32E−04 | | | | tryptase beta 1, tryptase beta | Hs.347933 |
| | | | | | | | 2, tryptase, alpha | |
| 204798_at | 368 | 12.95 | 7.41E−10 | 66.25% | 24.66% | MYB | v-myb myeloblastosis viral | Hs.1334 |
| | | | | | | | oncogene homolog (avian) | |
| 206851_at | 443 | 12.83 | 7.34E−03 | 194.31% | 50.67% | RNASE3 | ribonuclease, RNase A family, | Hs.73839 |
| | | | | | | | 3 (eosinophil cationic protein) | |
| 217023_x_at | 688 | 12.02 | 1.41E−04 | | | | tryptase beta 1, tryptase beta 2 | Hs.294158, |
| | | | | | | | | Hs.347933 |
| 216474_x_at | 681 | 11.06 | 8.25E−05 | | | | tryptase beta 1, tryptase beta 2 | Hs.347933 |
| 202016_at | 235 | 11.02 | 3.63E−04 | 138.17% | 24.92% | MEST | mesoderm specific transcript | Hs.79284 |
| | | | | | | | homolog (mouse) | |
| 207134_x_at | 447 | 10.94 | 6.98E−04 | 146.58% | 35.48% | TPS1, | tryptase beta 1, tryptase beta | Hs.294158 |
| | | | | | | TPSB1, | 2, tryptase, alpha | |
| | | | | | | TPSB2 | | |
| 215382_x_at | 670 | 10.85 | 5.25E−05 | | | | tryptase beta 1, tryptase, alpha | Hs.347933 |
| 205950_s_at | 420 | 10.85 | 5.23E−04 | | | CA1 | carbonic anhydrase I | Hs.23118 |
| 205051_s_at | 380 | 10.24 | 2.37E−05 | 111.13% | 30.96% | KIT | v-kit Hardy-Zuckerman 4 feline | Hs.81665 |
| | | | | | | | sarcoma viral oncogene | |
| | | | | | | | homolog | |
| 211709_s_at | 566 | 10.06 | 1.23E−06 | 92.43% | 24.57% | SCGF | stem cell growth factor; | Hs.425339, |
| | | | | | | | lymphocyte secreted C-type | Hs.105927 |
| | | | | | | | lectin | |
| 205131_x_at | 383 | 9.55 | 1.02E−04 | | | | stem cell growth factor; | Hs.105927 |
| | | | | | | | lymphocyte secreted C-type | |
| | | | | | | | lectin | |
| 219054_at | 753 | 8.32 | 2.05E−06 | | | FLJ14054 | hypothetical protein FLJ14054 | Hs.13528 |
| 204304_s_at | 340 | 7.69 | 4.74E−07 | 84.71% | 30.22% | PROML1 | prominin-like 1 (mouse) | Hs.112360 |
| 206674_at | 440 | 7.41 | 2.90E−07 | | | FLT3 | fms-related tyrosine kinase 3 | Hs.385 |
| 207741_x_at | 456 | 7.33 | 5.05E−05 | | | | tryptase, alpha | Hs.334455 |
| 202589_at | 257 | 7.08 | 1.63E−05 | 103.09% | 49.47% | TYMS | thymidylate synthetase | Hs.29475, |
| | | | | | | | | Hs.82962 |
| 210783_x_at | 549 | 6.99 | 5.96E−05 | 112.68% | 19.95% | SCGF | stem cell growth factor; | Hs.425339, |
| | | | | | | | lymphocyte secreted C-type | Hs.105927 |
| | | | | | | | lectin | |
| 211922_s_at | 576 | 6.71 | 1.13E−07 | 76.92% | 32.08% | CAT | catalase | Hs.395771, |
| | | | | | | | | Hs.76359 |
| 203373_at | 13 | 6.70 | 1.95E−02 | 208.35% | 23.04% | STATI2 | STAT induced STAT inhibitor-2 | Hs.405946 |
| 201427_s_at | 208 | 6.64 | 7.13E−04 | 137.31% | 0.00% | SEPP1 | selenoprotein P, plasma, 1 | Hs.275775, |
| | | | | | | | | Hs.3314 |
| 206111_at | 424 | 6.60 | 2.95E−05 | 106.04% | 41.83% | RNASE2 | ribonuclease, RNase A family, | Hs.728 |
| | | | | | | | 2 (liver, eosinophil-derived | |
| | | | | | | | neurotoxin) | |
| 213844_at | 638 | 6.60 | 2.86E−03 | 158.62% | 46.12% | HOXA5 | homeo box A5 | Hs.37034 |
| 202503_s_at | 255 | 6.39 | 2.92E−06 | | | KIAA0101 | KIAA0101 gene product | Hs.81892 |
| 205899_at | 417 | 6.26 | 1.91E−03 | 150.19% | 16.83% | CCNA1 | cyclin A1 | Hs.79378 |
| 220377_at | 778 | 6.14 | 1.93E−04 | 120.57% | 14.58% | HSPC053 | HSPC053 protein | Hs.128155 |
| 201310_s_at | 200 | 5.92 | 2.13E−09 | | | | P311 protein | Hs.142827 |
| 219672_at | 767 | 5.86 | 9.81E−04 | 137.79% | 96.37% | ERAF | erythroid associated factor | Hs.274309 |
| 208029_s_at | 58 | 5.69 | 2.37E−02 | 208.96% | 30.33% | LC27 | putative integral membrane | Hs.296398 |
| | | | | | | | transporter | |
| 205624_at | 409 | 5.66 | 9.30E−05 | 111.81% | 43.05% | CPA3 | carboxypeptidase A3 (mast | Hs.646 |
| | | | | | | | cell) | |
| 205609_at | 407 | 5.59 | 1.49E−06 | 85.15% | 34.40% | ANGPT1 | angiopoietin 1 | Hs.2463 |
| 206834_at | 442 | 5.49 | 5.46E−05 | 106.29% | 97.40% | HBD | hemoglobin, delta | Hs.36977 |
| 205557_at | 402 | 5.28 | 1.42E−02 | 188.13% | 75.52% | BPI | bactericidal/permeability- | Hs.89535 |
| | | | | | | | increasing protein | |
| 201162_at | 192 | 5.25 | 3.09E−07 | 76.99% | 53.67% | IGFBP7 | insulin-like growth factor | Hs.119206 |
| | | | | | | | binding protein 7 | |
| 201432_at | 209 | 5.18 | 1.43E−09 | | | | catalase | Hs.76359 |
| 204430_s_at | 8 | 5.17 | 6.73E−04 | 129.63% | 30.33% | SLC2A5 | solute carrier family 2 | Hs.33084 |
| | | | | | | | (facilitated glucose/fructose | |
| | | | | | | | transporter), member 5 | |
| 220416_at | 780 | 5.16 | 1.24E−06 | 82.78% | 18.42% | KIAA1939 | KIAA1939 protein | Hs.182738 |
| 204030_s_at | 322 | 5.06 | 2.43E−03 | 147.20% | 34.79% | SCHIP1 | schwannomin interacting | Hs.61490 |
| | | | | | | | protein 1 | |
| 211743_s_at | 568 | 4.95 | 7.28E−04 | 129.14% | 32.90% | PRG2 | proteoglycan 2, bone marrow | Hs.99962 |
| | | | | | | | (natural killer cell activator, | |
| | | | | | | | eosinophil granule major basic | |
| | | | | | | | protein) | |
| 201416_at | 206 | 4.94 | 1.01E−04 | 109.06% | 35.67% | MEIS3, | Meis1, myeloid ecotropic viral | Hs.83484 |
| | | | | | | SOX4 | integration site 1 homolog 3 | |
| | | | | | | | (mouse), SRY (sex | |
| | | | | | | | determining region Y)-box 4 | |
| 213150_at | 617 | 4.90 | 3.44E−04 | 120.37% | 26.79% | HOXA10 | homeo box A10 | Hs.110637 |
| 209543_s_at | 502 | 4.88 | 6.90E−07 | 78.99% | 30.30% | CD34, | CD34 antigen, FLJ00005 | Hs.374990 |
| | | | | | | FLJ00005 | protein | |
| 213258_at | 65 | 4.82 | 2.40E−07 | | | | | Hs.288582 |
| 216667_at | 684 | 4.79 | 3.15E−03 | 149.58% | 27.72% | | | |
| 210664_s_at | 546 | 4.73 | 8.77E−06 | 90.93% | 34.92% | TFPI | tissue factor pathway inhibitor | Hs.170279 |
| | | | | | | | (lipoprotein-associated | |
| | | | | | | | coagulation inhibitor) | |
| 206067_s_at | 423 | 4.72 | 2.81E−04 | | | WT1 | Wilms tumor 1 | Hs.1145 |
| 209757_s_at | 70 | 4.69 | 8.72E−06 | 90.78% | 0.00% | MYCN | v-myc myelocytomatosis viral | Hs.25960 |
| | | | | | | | related oncogene, | |
| | | | | | | | neuroblastoma derived (avian) | |
| 213515_x_at | 629 | 4.68 | 2.22E−05 | 95.77% | 91.95% | GARS, | glycyl-tRNA synthetase, | Hs.356717, |
| | | | | | | HBG1, | hemoglobin, gamma A, | Hs.283108 |
| | | | | | | HBG2 | hemoglobin, gamma G | |
| 219837_s_at | 769 | 4.60 | 2.68E−04 | 115.74% | 34.92% | C17 | cytokine-like protein C17 | Hs.13872 |
| 218899_s_at | 746 | 4.57 | 9.36E−04 | 129.54% | 35.71% | BAALC | brain and acute leukemia, | Hs.169395 |
| | | | | | | | cytoplasmic | |
| 210665_at | 75 | 4.55 | 5.86E−05 | 102.39% | 28.60% | TFPI | tissue factor pathway inhibitor | Hs.170279 |
| | | | | | | | (lipoprotein-associated | |
| | | | | | | | coagulation inhibitor) | |
| 206478_at | 436 | 4.52 | 1.57E−04 | 110.17% | 39.54% | KIAA0125 | KIAA0125 gene product | Hs.38365 |
| 201825_s_at | 226 | 4.51 | 2.04E−07 | 72.49% | 26.57% | LOC51097 | CGI-49 protein | Hs.238126 |
| 202441_at | 252 | 4.46 | 3.52E−09 | 59.64% | 32.71% | KEO4 | similar to Caenorhabditis | Hs.285818 |
| | | | | | | | elegans protein C42C1.9 | |
| 209771_x_at | 510 | 4.43 | 3.13E−02 | 206.78% | 65.40% | CD24 | CD24 antigen (small cell lung | Hs.375108 |
| | | | | | | | carcinoma cluster 4 antigen) | |
| 209160_at | 484 | 4.38 | 3.56E−04 | 116.99% | 34.40% | AKR1C3 | aldo-keto reductase family 1, | Hs.78183 |
| | | | | | | | member C3 (3-alpha | |
| | | | | | | | hydroxysteroid | |
| | | | | | | | dehydrogenase, type II) | |
| 216379_x_at | 680 | 4.38 | 2.65E−02 | 199.51% | 62.52% | CD24, | CD24 antigen (small cell lung | Hs.381004 |
| | | | | | | G22P1, | carcinoma cluster 4 antigen), | |
| | | | | | | KIAA1919 | KIAA1919 protein, thyroid | |
| | | | | | | | autoantigen 70 kD (Ku antigen) | |
| 206207_at | 427 | 4.35 | 3.42E−02 | 209.28% | 70.13% | CLC | Charot-Leyden crystal protein | Hs.889 |
| 204561_x_at | 353 | 4.33 | 1.62E−02 | 182.63% | 0.00% | APOC2 | apolipoprotein C-II | Hs.75615 |
| 203372_s_at | 16 | 4.33 | 4.22E−02 | 218.85% | 18.42% | STATI2 | STAT induced STAT inhibitor-2 | Hs.405946 |
| 207269_at | 448 | 4.30 | 9.46E−03 | 167.00% | 84.09% | DEFA4 | defensin, alpha 4, corticostatin | Hs.2582 |
| 218788_s_at | 735 | 4.30 | 3.35E−06 | 83.45% | 19.69% | FLJ21080 | hypothetical protein FLJ21080 | Hs.8109 |
| 211821_x_at | 572 | 4.25 | 1.03E−03 | 128.12% | 31.72% | GYPA | glycophorin A (includes MN | Hs.108694 |
| | | | | | | | blood group) | |
| 204419_x_at | 347 | 4.25 | 5.06E−05 | 98.31% | 100.03% | GARS, | glycyl-tRNA synthetase, | Hs.386655 |
| | | | | | | HBG1, | hemoglobin, gamma A, | |
| | | | | | | HBG2 | hemoglobin, gamma G | |
| 213147_at | 616 | 4.19 | 2.64E−05 | 94.35% | 37.81% | HOXA10 | homeo box A10 | Hs.110637 |
| 221004_s_at | 792 | 4.11 | 7.39E−06 | 86.29% | 36.24% | ITM3 | integral membrane protein 3 | Hs.111577 |
| 204848_x_at | 371 | 4.09 | 5.66E−05 | 97.77% | 101.47% | HBG1, | hemoglobin, gamma A, | Hs.283108 |
| | | | | | | HBG2 | hemoglobin, gamma G | |
| 211560_s_at | 563 | 4.08 | 9.01E−03 | 159.47% | 191.88% | ALAS2 | aminolevulinate, delta-, | Hs.381218 |
| | | | | | | | synthase 2 | |
| | | | | | | | (sideroblastic/hypochromic | |
| | | | | | | | anemia) | |
| 206135_at | 425 | 4.00 | 4.98E−02 | 221.44% | 0.00% | ZNF387 | zinc finger protein 387 | Hs.151449 |
| 205366_s_at | 6 | 3.87 | 2.03E−04 | 107.19% | 30.33% | HOXB6 | homeo box B6 | Hs.98428 |
| 213110_s_at | 612 | 3.87 | 2.06E−05 | 90.35% | 32.83% | COL4A5 | collagen, type IV, alpha 5 | Hs.169825 |
| | | | | | | | (Alport syndrome) | |
| 219654_at | 766 | 3.85 | 1.23E−06 | 75.89% | 35.75% | PTPLA | protein tyrosine phosphatase- | Hs.114062 |
| | | | | | | | like (proline instead of catalytic | |
| | | | | | | | arginine), member a | |
| 201596_x_at | 222 | 3.84 | 1.13E−03 | 125.06% | 18.96% | KRT18 | keratin 18 | Hs.406013 |
| 220232_at | 776 | 3.82 | 2.74E−07 | 69.76% | 30.96% | FLJ21032 | hypothetical protein FLJ21032 | Hs.379191 |
| 207341_at | 450 | 3.77 | 2.42E−03 | 134.65% | 33.45% | PRTN3 | proteinase 3 (serine | Hs.928 |
| | | | | | | | proteinase, neutrophil, | |
| | | | | | | | Wegener granulomatosis | |
| | | | | | | | autoantigen) | |
| 210746_s_at | 547 | 3.73 | 7.35E−03 | 151.59% | 136.15% | EPB42 | erythrocyte membrane protein | Hs.733 |
| | | | | | | | band 4.2 | |
| 201892_s_at | 229 | 3.71 | 7.86E−08 | 64.85% | 33.27% | IMPDH2 | IMP (inosine monophosphate) | Hs.75432 |
| | | | | | | | dehydrogenase 2 | |
| 214433_s_at | 652 | 3.70 | 8.36E−03 | 153.06% | 158.09% | SELENBP1 | selenium binding protein 1 | Hs.334841 |
| 218718_at | 734 | 3.70 | 1.78E−06 | 76.48% | 21.46% | PDGFC | platelet derived growth factor C | Hs.43080 |
| 213479_at | 627 | 3.64 | 2.60E−02 | 187.19% | 14.58% | NPTX2 | neuronal pentraxin II | Hs.3281 |
| 201459_at | 210 | 3.61 | 4.46E−07 | 70.09% | 40.13% | RUVBL2 | RuvB-like 2 (E. coli) | Hs.6455 |
| 218313_s_at | 720 | 3.60 | 6.70E−07 | 71.60% | 22.51% | GALNT7 | UDP-N-acetyl-alpha-D- | Hs.246315 |
| | | | | | | | galactosamine:polypeptide N- | |
| | | | | | | | acetylgalactosaminyltransferase | |
| | | | | | | | 7 (GalNAc-T7) | |
| 207459_x_at | 451 | 3.59 | 3.58E−05 | 91.28% | 28.85% | GYPA, | glycophorin A (includes MN | Hs.372513 |
| | | | | | | GYPB | blood group), glycophorin B | |
| | | | | | | | (includes Ss blood group) | |
| 214407_x_at | 651 | 3.58 | 2.91E−04 | 107.39% | 22.02% | GYPA, | glycophorin A (includes MN | Hs.372513 |
| | | | | | | GYPB | blood group), glycophorin B | |
| | | | | | | | (includes Ss blood group) | |
| 202502_at | 254 | 3.58 | 1.42E−07 | 65.88% | 20.33% | ACADM | acyl-Coenzyme A | Hs.79158 |
| | | | | | | | dehydrogenase, C-4 to C-12 | |
| | | | | | | | straight chain | |
| 201418_s_at | 207 | 3.55 | 7.35E−07 | 71.24% | 61.97% | MEIS3, | Meis1, myeloid ecotropic viral | Hs.83484 |
| | | | | | | SOX4 | integration site 1 homolog 3 | |
| | | | | | | | (mouse), SRY (sex | |
| | | | | | | | determining region Y)-box 4 | |
| 209790_s_at | 512 | 3.49 | 4.47E−05 | 91.75% | 25.40% | CASP6 | caspase 6, apoptosis-related | Hs.3280 |
| | | | | | | | cysteine protease | |
| 204069_at | 325 | 3.48 | 3.01E−04 | 106.42% | 25.85% | MEIS1 | Meis1, myeloid ecotropic viral | Hs.170177 |
| | | | | | | | integration site 1 homolog | |
| | | | | | | | (mouse) | |
| 203502_at | 295 | 3.46 | 5.36E−04 | 110.86% | 77.38% | BPGM | 2,3-bisphosphoglycerate | Hs.198365 |
| | | | | | | | mutase | |
| 206726_at | 441 | 3.45 | 9.57E−03 | 155.35% | 30.96% | PGDS | prostaglandin D2 synthase, | Hs.128433 |
| | | | | | | | hematopoietic | |
| 209813_x_at | 513 | 3.42 | 9.06E−04 | 116.74% | 46.61% | TRG@ | T cell receptor gamma locus | Hs.112259 |
| 218332_at | 721 | 3.40 | 1.19E−02 | 159.40% | 27.69% | BEX1 | brain expressed, X-linked 1 | Hs.334370 |
| 219218_at | 757 | 3.37 | 2.70E−05 | 87.16% | 34.79% | FLJ23058 | hypothetical protein FLJ23058 | Hs.98968 |
| 211144_x_at | 556 | 3.37 | 1.07E−03 | 117.91% | 41.76% | TRG@ | T cell receptor gamma locus | Hs.112259 |
| 202444_s_at | 253 | 3.31 | 2.44E−10 | 47.88% | 12.86% | KEO4 | similar to Caenorhabditis | Hs.285818 |
| | | | | | | | elegans protein C42C1.9 | |
| 201193_at | 194 | 3.29 | 4.31E−05 | 89.35% | 22.26% | IDH1 | isocitrate dehydrogenase 1 | Hs.11223 |
| | | | | | | | (NADP+), soluble | |
| 212175_s_at | 587 | 3.28 | 2.59E−08 | 58.54% | 25.74% | AK2 | adenylate kinase 2 | Hs.334802 |
| 205513_at | 400 | 3.28 | 1.70E−03 | 122.27% | 42.32% | TCN1 | transcobalamin I (vitamin B12 | Hs.2012 |
| | | | | | | | binding protein, R binder | |
| | | | | | | | family) | |
| 205592_at | 403 | 3.25 | 3.97E−03 | 131.52% | 121.76% | SLC4A1 | solute carrier family 4, anion | Hs.432645 |
| | | | | | | | exchanger, member 1 | |
| | | | | | | | (erythrocyte membrane protein | |
| | | | | | | | band 3, Diego blood group) | |
| 205769_at | 413 | 3.24 | 1.32E−05 | 81.73% | 33.71% | FACVL1 | fatty-acid-Coenzyme A ligase, | Hs.11729 |
| | | | | | | | very long-chain 1 | |
| 212141_at | 586 | 3.19 | 7.85E−05 | 92.20% | 0.00% | MCM4 | MCM4 minichromosome | Hs.154443 |
| | | | | | | | maintenance deficient 4 (S. cerevisiae) | |
| 213541_s_at | 631 | 3.17 | 2.40E−09 | 51.84% | 32.90% | ERG | v-ets erythroblastosis virus | Hs.45514 |
| | | | | | | | E26 oncogene like (avian) | |
| 204468_s_at | 9 | 3.17 | 1.48E−02 | 160.05% | 0.00% | TIE | tyrosine kinase with | Hs.78824 |
| | | | | | | | immunoglobulin and epidermal | |
| | | | | | | | growth factor homology | |
| | | | | | | | domains | |
| 222036_s_at | 807 | 3.16 | 1.44E−04 | 96.14% | 7.37% | MCM4 | MCM4 minichromosome | Hs.319215 |
| | | | | | | | maintenance deficient 4 (S. cerevisiae) | |
| 220668_s_at | 39 | 3.15 | 2.45E−07 | 64.13% | 20.33% | DNMT3B | DNA (cytosine-5-)- | Hs.251673 |
| | | | | | | | methyltransferase 3 beta | |
| 218847_at | 741 | 3.15 | 2.96E−12 | 40.44% | 50.24% | IMP-2 | IGF-II mRNA-binding protein 2 | Hs.30299 |
| 217294_s_at | 691 | 3.14 | 2.68E−08 | 57.40% | 44.65% | ENO1 | enolase 1, (alpha) | Hs.381397 |
| 213779_at | 636 | 3.12 | 5.52E−07 | 66.61% | 27.57% | LOC129080 | putative emu1 | Hs.289106 |
| 218825_at | 738 | 3.12 | 7.45E−07 | 67.61% | 35.39% | LOC51162 | NEU1 protein | Hs.91481 |
| 218858_at | 743 | 3.09 | 1.82E−05 | 81.78% | 17.08% | FLJ12428 | hypothetical protein FLJ12428 | Hs.87729 |
| 216153_x_at | 676 | 3.08 | 8.64E−06 | 77.60% | 35.89% | RECK | reversion-inducing-cysteine- | Hs.29640 |
| | | | | | | | rich protein with kazal motifs | |
| 204467_s_at | 351 | 3.08 | 3.20E−02 | 176.33% | 158.31% | SNCA | synuclein, alpha (non A4 | Hs.76930 |
| | | | | | | | component of amyloid | |
| | | | | | | | precursor) | |
| 204409_s_at | 345 | 3.08 | 8.03E−04 | 109.25% | 66.65% | EIF1AY | eukaryotic translation initiation | Hs.155103 |
| | | | | | | | factor 1A, Y chromosome | |
| 205202_at | 384 | 3.05 | 2.34E−05 | 82.67% | 22.02% | PCMT1 | protein-L-isoaspartate (D- | Hs.79137 |
| | | | | | | | aspartate) O- | |
| | | | | | | | methyltransferase | |
| 205382_s_at | 394 | 3.05 | 2.83E−05 | 83.59% | 34.99% | DF | D component of complement | Hs.155597 |
| | | | | | | | (adipsin) | |
| 209576_at | 503 | 3.04 | 7.79E−04 | 109.41% | 14.58% | GNAI1 | guanine nucleotide binding | Hs.203862 |
| | | | | | | | protein (G protein), alpha | |
| | | | | | | | inhibiting activity polypeptide 1 | |
| 211546_x_at | 562 | 3.03 | 6.29E−03 | 136.16% | 91.15% | SNCA | synuclein, alpha (non A4 | Hs.76930 |
| | | | | | | | component of amyloid | |
| | | | | | | | precursor) | |
| 212115_at | 585 | 3.02 | 4.78E−04 | 103.69% | 45.78% | FLJ13092 | hypothetical protein FLJ13092 | Hs.172035 |
| 211820_x_at | 571 | 3.01 | 6.29E−04 | 106.39% | 33.71% | GYPA | glycophorin A (includes MN | Hs.108694 |
| | | | | | | | blood group) | |
| 210254_at | 530 | 2.98 | 6.65E−03 | 137.19% | 59.25% | MS4A3 | membrane-spanning 4- | Hs.99960 |
| | | | | | | | domains, subfamily A, member | |
| | | | | | | | 3 (hematopoietic cell-specific) | |
| 210829_s_at | 550 | 2.97 | 2.80E−05 | 82.60% | 20.75% | SSBP2 | single-stranded DNA binding | Hs.424652 |
| | | | | | | | protein 2 | |
| 200923_at | 177 | 2.97 | 1.47E−04 | 93.21% | 32.12% | LGALS3BP | lectin, galactoside-binding, | Hs.79339 |
| | | | | | | | soluble, 3 binding protein | |
| 204900_x_at | 375 | 2.96 | 1.38E−04 | 92.64% | 31.39% | SAP30 | sin3-associated polypeptide, | Hs.20985 |
| | | | | | | | 30 kD | |
| 202845_s_at | 268 | 2.95 | 1.36E−07 | 59.80% | 60.88% | RALBP1 | ralA binding protein 1 | Hs.75447 |
| 203787_at | 307 | 2.94 | 3.89E−05 | 83.97% | 20.55% | SSBP2 | single-stranded DNA binding | Hs.169833 |
| | | | | | | | protein 2 | |
| 206622_at | 437 | 2.93 | 4.83E−02 | 193.09% | 26.43% | TRH | thyrotropin-releasing hormone | Hs.182231 |
| 201413_at | 205 | 2.93 | 5.86E−08 | 57.63% | 26.79% | HSD17B4 | hydroxysteroid (17-beta) | Hs.75441 |
| | | | | | | | dehydrogenase 4 | |
| 201054_at | 189 | 2.91 | 2.70E−07 | 62.01% | 29.74% | HNRPA0 | heterogeneous nuclear | Hs.77492 |
| | | | | | | | ribonucleoprotein A0 | |
| 204647_at | 360 | 2.90 | 2.54E−04 | 96.25% | 29.14% | HOMER-3 | Homer, neuronal immediate | Hs.424053 |
| | | | | | | | early gene, 3 | |
| 219789_at | 768 | 2.89 | 4.95E−06 | 72.67% | 26.79% | NPR3 | natriuretic peptide receptor | Hs.123655 |
| | | | | | | | C/guanylate cyclase C | |
| | | | | | | | (atrionatriuretic peptide | |
| | | | | | | | receptor C) | |
| 204011_at | 319 | 2.88 | 7.38E−04 | 105.71% | 21.81% | SPRY2 | sprouty homolog 2 | Hs.18676 |
| | | | | | | | (Drosophila) | |
| 204391_x_at | 343 | 2.87 | 4.74E−11 | 42.14% | 25.33% | TIF1 | transcriptional intermediary | Hs.183858 |
| | | | | | | | factor 1 | |
| 205844_at | 415 | 2.85 | 9.58E−03 | 141.91% | 32.83% | VNN1 | vanin 1 | Hs.12114 |
| 209183_s_at | 485 | 2.85 | 1.07E−03 | 108.94% | 19.95% | DEPP | decidual protein induced by | Hs.93675 |
| | | | | | | | progesterone | |
| 214657_s_at | 661 | 2.82 | 1.23E−06 | 66.05% | 31.54% | MEN1 | multiple endocrine neoplasia I | Hs.434021 |
| 200615_s_at | 157 | 2.81 | 6.19E−08 | 56.39% | 39.24% | AP2B1 | adaptor-related protein | Hs.74626 |
| | | | | | | | complex 2, beta 1 subunit | |
| 204466_s_at | 350 | 2.80 | 1.14E−02 | 141.03% | 106.77% | SNCA | synuclein, alpha (non A4 | Hs.76930 |
| | | | | | | | component of amyloid | |
| | | | | | | | precursor) | |
| 215537_x_at | 672 | 2.80 | 1.10E−06 | 65.18% | 41.33% | DDAH2 | dimethylarginine | Hs.247362 |
| | | | | | | | dimethylaminohydrolase 2 | |
| 206480_at | 66 | 2.79 | 4.45E−05 | 82.52% | 19.95% | LTC4S | leukotriene C4 synthase | Hs.456 |
| 222067_x_at | 809 | 2.77 | 5.86E−06 | 71.70% | 31.83% | H2BFB | H2B histone family, member B | Hs.180779 |
| 204173_at | 333 | 2.77 | 4.04E−12 | 37.74% | 23.97% | MLC1SA | myosin light chain 1 slow a | Hs.90318 |
| 204885_s_at | 372 | 2.77 | 2.56E−02 | 164.20% | 19.95% | MSLN | mesothelin | Hs.155981 |
| 212268_at | 593 | 2.75 | 5.30E−08 | 55.45% | 22.18% | SERPINB1 | serine (or cysteine) proteinase | Hs.183583 |
| | | | | | | | inhibitor, clade B (ovalbumin), | |
| | | | | | | | member 1 | |
| 215182_x_at | 667 | 2.75 | 2.81E−08 | 53.77% | 25.51% | | | Hs.274511 |
| 201037_at | 188 | 2.75 | 1.97E−06 | 66.97% | 23.73% | PFKP | phosphofructokinase, platelet | Hs.99910 |
| 205900_at | 418 | 2.75 | 2.10E−02 | 151.32% | 152.69% | KRT1 | keratin 1 (epidermolytic | Hs.80828 |
| | | | | | | | hyperkeratosis) | |
| 214236_at | 648 | 2.74 | 4.55E−04 | 98.32% | 26.79% | | | Hs.343877 |
| 210644_s_at | 544 | 2.74 | 4.64E−08 | 54.96% | 29.13% | LAIR1 | leukocyte-associated Ig-like | Hs.115808 |
| | | | | | | | receptor 1 | |
| 201563_at | 217 | 2.73 | 1.24E−06 | 64.94% | 22.33% | SORD | sorbitol dehydrogenase | Hs.878 |
| 210395_x_at | 535 | 2.72 | 1.04E−02 | 139.39% | 52.16% | MYL4 | myosin, light polypeptide 4, | Hs.356717 |
| | | | | | | | alkali; atrial, embryonic | |
| 213301_x_at | 621 | 2.72 | 5.42E−10 | 45.00% | 23.44% | TIF1 | transcriptional intermediary | Hs.183858 |
| | | | | | | | factor 1 | |
| 218039_at | 709 | 2.71 | 1.12E−06 | 64.37% | 23.77% | ANKT | nucleolar protein ANKT | Hs.279905 |
| 218069_at | 710 | 2.70 | 1.77E−05 | 75.65% | 39.91% | MGC5627 | hypothetical protein MGC5627 | Hs.237971 |
| 203588_s_at | 300 | 2.69 | 2.26E−06 | 66.62% | 29.27% | TFDP2 | transcription factor Dp-2 (E2F | Hs.379018 |
| | | | | | | | dimerization partner 2) | |
| 218883_s_at | 745 | 2.68 | 1.49E−05 | 74.69% | 22.08% | FLJ23468 | hypothetical protein FLJ23468 | Hs.38178 |
| 209360_s_at | 493 | 2.67 | 3.42E−07 | 59.70% | 35.04% | RUNX1 | runt-related transcription factor | Hs.129914 |
| | | | | | | | 1 (acute myeloid leukemia 1; | |
| | | | | | | | aml1 oncogene) | |
| 201503_at | 212 | 2.66 | 4.32E−05 | 80.08% | 23.20% | G3BP | Ras-GTPase-activating protein | Hs.220689 |
| | | | | | | | SH3-domain-binding protein | |
| 200696_s_at | 160 | 2.65 | 2.10E−08 | 51.86% | 26.02% | GSN | gelsolin (amyloidosis, Finnish | Hs.290070 |
| | | | | | | | type) | |
| 216054_x_at | 675 | 2.63 | 6.99E−03 | 128.94% | 51.23% | MYL4 | myosin, light polypeptide 4, | Hs.433562 |
| | | | | | | | alkali; atrial, embryonic | |
| 218342_s_at | 722 | 2.62 | 1.78E−08 | 51.17% | 29.01% | FLJ23309 | hypothetical protein FLJ23309 | Hs.87128 |
| 209825_s_at | 514 | 2.62 | 1.18E−07 | 55.95% | 20.26% | UMPK | uridine monophosphate kinase | Hs.95734 |
| 217975_at | 24 | 2.60 | 3.93E−05 | 78.27% | 30.22% | LOC51186 | pp21 homolog | Hs.15984 |
| 217791_s_at | 697 | 2.60 | 3.00E−08 | 52.16% | 27.47% | PYCS | pyrroline-5-carboxylate | Hs.114366 |
| | | | | | | | synthetase (glutamate | |
| | | | | | | | gamma-semialdehyde | |
| | | | | | | | synthetase) | |
| 203662_s_at | 302 | 2.60 | 3.81E−03 | 115.58% | 96.82% | TMOD | tropomodulin | Hs.374849 |
| 208967_s_at | 481 | 2.59 | 1.23E−09 | 45.20% | 19.58% | AK2 | adenylate kinase 2 | Hs.294008 |
| 202371_at | 249 | 2.59 | 4.15E−06 | 67.51% | 23.93% | FLJ21174 | hypothetical protein FLJ21174 | Hs.194329 |
| 212055_at | 583 | 2.59 | 1.69E−06 | 63.82% | 35.39% | DKFZP586M1523 | DKFZP586M1523 protein | Hs.22981 |
| 200703_at | 161 | 2.58 | 6.22E−05 | 80.36% | 34.35% | PIN | dynein, cytoplasmic, light | Hs.5120 |
| | | | | | | | polypeptide | |
| 202262_x_at | 245 | 2.57 | 1.20E−07 | 55.38% | 30.08% | DDAH2 | dimethylarginine | Hs.247362 |
| | | | | | | | dimethylaminohydrolase 2 | |
| 209200_at | 487 | 2.56 | 5.08E−04 | 95.07% | 35.56% | MEF2C | MADS box transcription | Hs.78995 |
| | | | | | | | enhancer factor 2, polypeptide | |
| | | | | | | | C (myocyte enhancer factor | |
| | | | | | | | 2C) | |
| 213572_s_at | 632 | 2.56 | 6.00E−07 | 60.04% | 24.71% | SERPINB1 | serine (or cysteine) proteinase | Hs.183583 |
| | | | | | | | inhibitor, clade B (ovalbumin), | |
| | | | | | | | member 1 | |
| 210762_s_at | 548 | 2.56 | 1.07E−04 | 83.59% | 21.67% | DLC1 | deleted in liver cancer 1 | Hs.8700 |
| 200658_s_at | 159 | 2.56 | 1.37E−06 | 62.62% | 33.60% | PHB | prohibitin | Hs.75323 |
| 201325_s_at | 202 | 2.56 | 1.02E−03 | 101.41% | 34.91% | EMP1 | epithelial membrane protein 1 | Hs.79368 |
| 210999_s_at | 554 | 2.56 | 4.21E−06 | 67.09% | 10.66% | GRB10 | growth factor receptor-bound | Hs.81875 |
| | | | | | | | protein 10 | |
| 205518_s_at | 401 | 2.55 | 7.90E−09 | 48.51% | 21.91% | CMAH | cytidine monophosphate-N- | |
| | | | | | | | acetylneuraminic acid | |
| | | | | | | | hydroxylase (CMP-N- | |
| | | | | | | | acetylneuraminate | |
| | | | | | | | monooxygenase) | |
| 217809_at | 698 | 2.55 | 6.77E−09 | 48.13% | 20.59% | HSPC028 | HSPC028 protein | Hs.5216 |
| 210088_x_at | 526 | 2.54 | 1.55E−02 | 142.11% | 53.21% | MYL4 | myosin, light polypeptide 4, | Hs.433562 |
| | | | | | | | alkali; atrial, embryonic | |
| 220725_x_at | 785 | 2.54 | 1.18E−07 | 54.83% | 20.23% | FLJ23558 | hypothetical protein FLJ23558 | Hs.288552 |
| 208857_s_at | 472 | 2.54 | 7.84E−06 | 69.20% | 24.21% | PCMT1 | protein-L-isoaspartate (D- | Hs.79137 |
| | | | | | | | aspartate) O- | |
| | | | | | | | methyltransferase | |
| 210401_at | 536 | 2.53 | 1.55E−09 | 45.09% | 36.41% | P2RX1 | purinergic receptor P2X, | Hs.41735 |
| | | | | | | | ligand-gated ion channel, 1 | |
| 201555_at | 215 | 2.53 | 9.94E−06 | 70.17% | 23.11% | MCM3 | MCM3 minichromosome | Hs.179565 |
| | | | | | | | maintenance deficient 3 (S. cerevisiae) | |
| 202708_s_at | 260 | 2.53 | 1.43E−04 | 84.55% | 34.53% | H2BFQ | H2B histone family, member Q | Hs.2178 |
| 208651_x_at | 464 | 2.53 | 2.33E−02 | 151.82% | 55.28% | CD24 | CD24 antigen (small cell lung | Hs.375108 |
| | | | | | | | carcinoma cluster 4 antigen) | |
| 201951_at | 230 | 2.52 | 5.47E−05 | 78.34% | 35.71% | ALCAM | activated leucocyte cell | Hs.10247 |
| | | | | | | | adhesion molecule | |
| 201564_s_at | 218 | 2.52 | 9.43E−05 | 81.60% | 35.59% | SNL | singed-like (fascin homolog, | Hs.118400 |
| | | | | | | | sea urchin) (Drosophila) | |
| 220807_at | 787 | 2.51 | 1.86E−02 | 142.62% | 100.98% | HBQ1 | hemoglobin, theta 1 | Hs.247921 |
| 201005_at | 183 | 2.51 | 1.68E−03 | 104.10% | 68.43% | CD9 | CD9 antigen (p24) | Hs.1244 |
| 205801_s_at | 414 | 2.50 | 5.77E−03 | 121.93% | 35.56% | GRP3 | guanine nucleotide exchange | Hs.24024 |
| | | | | | | | factor for Rap1 | |
| 221521_s_at | 797 | 2.50 | 6.08E−03 | 123.19% | 14.58% | LOC51659 | HSPC037 protein | Hs.433180 |
| 208690_s_at | 467 | 2.50 | 5.11E−07 | 58.47% | 25.48% | PDLIM1 | PDZ and LIM domain 1 (elfin) | Hs.75807 |
| 201015_s_at | 184 | 2.48 | 1.26E−04 | 81.37% | 61.73% | JUP | junction plakoglobin | Hs.2340 |
| 203661_s_at | 301 | 2.47 | 4.13E−03 | 114.18% | 73.79% | TMOD | tropomodulin | Hs.374849 |
| 266_s_at | 814 | 2.46 | 3.21E−02 | 159.03% | 38.81% | CD24 | CD24 antigen (small cell lung | Hs.375108 |
| | | | | | | | carcinoma cluster 4 antigen) | |
| 209409_at | 496 | 2.46 | 2.57E−06 | 63.47% | 10.66% | GRB10 | growth factor receptor-bound | Hs.81875 |
| | | | | | | | protein 10 | |
| 203560_at | 299 | 2.46 | 1.44E−04 | 83.27% | 16.83% | GGH | gamma-glutamyl hydrolase | Hs.78619 |
| | | | | | | | (conjugase, | |
| | | | | | | | folylpolygammaglutamyl | |
| | | | | | | | hydrolase) | |
| 213170_at | 618 | 2.45 | 5.82E−10 | 42.28% | 21.81% | CL683 | weakly similar to glutathione | Hs.43728 |
| | | | | | | | peroxidase 2 | |
| 205227_at | 11 | 2.45 | 6.61E−05 | 77.91% | 32.30% | IL1RAP | interleukin 1 receptor | Hs.173880 |
| | | | | | | | accessory protein | |
| 218927_s_at | 747 | 2.44 | 1.69E−05 | 70.44% | 42.51% | C4S-2 | chondroitin 4-O- | Hs.25204 |
| | | | | | | | sulfotransferase 2 | |
| 209318_x_at | 492 | 2.44 | 7.63E−06 | 67.41% | 20.62% | PLAGL1 | pleiomorphic adenoma gene- | Hs.75825 |
| | | | | | | | like 1 | |
| 214106_s_at | 645 | 2.43 | 4.48E−03 | 116.13% | 23.65% | GMDS | GDP-mannose 4,6- | Hs.105435 |
| | | | | | | | dehydratase | |
| 213346_at | 623 | 2.43 | 8.55E−06 | 67.73% | 20.13% | LOC93081 | hypothetical protein BC015148 | Hs.13413 |
| 205418_at | 395 | 2.43 | 2.60E−04 | 86.33% | 37.54% | FES | feline sarcoma oncogene | Hs.7636 |
| 220051_at | 773 | 2.43 | 2.32E−02 | 148.56% | 15.25% | PRSS21 | protease, serine, 21 (testisin) | Hs.72026 |
| 202107_s_at | 239 | 2.43 | 8.20E−05 | 78.99% | 21.20% | MCM2 | MCM2 minichromosome | Hs.57101 |
| | | | | | | | maintenance deficient 2, | |
| | | | | | | | mitotin (S. cerevisiae) | |
| 202862_at | 271 | 2.42 | 3.03E−07 | 55.80% | 20.78% | FAH | fumarylacetoacetate hydrolase | Hs.73875 |
| | | | | | | | (fumarylacetoacetase) | |
| 204086_at | 327 | 2.42 | 4.35E−02 | 167.93% | 24.76% | PRAME | preferentially expressed | Hs.30743 |
| | | | | | | | antigen in melanoma | |
| 212526_at | 601 | 2.42 | 2.71E−06 | 62.96% | 7.37% | KIAA0610 | KIAA0610 protein | Hs.118087 |
| 210358_x_at | 533 | 2.42 | 1.91E−06 | 61.37% | 32.70% | GATA2, | GATA binding protein 2, | Hs.760 |
| | | | | | | MGC2306 | hypothetical protein MGC2306 | |
| 220615_s_at | 782 | 2.41 | 7.40E−04 | 94.63% | 30.22% | FLJ10462 | hypothetical protein FLJ10462 | Hs.100895 |
| 205612_at | 408 | 2.40 | 3.50E−02 | 159.14% | 23.65% | MMRN | multimerin | Hs.268107 |
| 200648_s_at | 158 | 2.39 | 5.01E−04 | 89.77% | 52.01% | GLUL | glutamate-ammonia ligase | Hs.170171 |
| | | | | | | | (glutamine synthase) | |
| 201277_s_at | 198 | 2.39 | 4.92E−06 | 64.59% | 19.32% | HNRPAB | heterogeneous nuclear | Hs.81361 |
| | | | | | | | ribonucleoprotein A/B | |
| 210044_s_at | 522 | 2.39 | 2.22E−09 | 43.75% | 45.66% | LYL1 | lymphoblastic leukemia | Hs.46446 |
| | | | | | | | derived sequence 1 | |
| 214501_s_at | 656 | 2.38 | 2.15E−08 | 48.45% | 21.49% | H2AFY | H2A histone family, member Y | Hs.75258 |
| 201240_s_at | 196 | 2.37 | 6.69E−07 | 56.91% | 36.63% | KIAA0102 | KIAA0102 gene product | Hs.77665 |
| 208626_s_at | 463 | 2.36 | 2.87E−08 | 48.71% | 24.12% | VATI | vesicle amine transport protein 1 | Hs.157236 |
| 205349_at | 393 | 2.35 | 2.52E−05 | 70.03% | 46.83% | GNA15 | guanine nucleotide binding | Hs.73797 |
| | | | | | | | protein (G protein), alpha 15 | |
| | | | | | | | (Gq class) | |
| 216833_x_at | 686 | 2.35 | 4.00E−04 | 87.94% | 12.86% | GYPB, | glycophorin B (includes Ss | Hs.372513 |
| | | | | | | GYPE | blood group), glycophorin E | |
| 218026_at | 707 | 2.34 | 5.33E−06 | 63.97% | 21.95% | HSPC009 | HSPC009 protein | Hs.16059 |
| 211464_x_at | 560 | 2.34 | 2.51E−06 | 60.85% | 35.12% | CASP6 | caspase 6, apoptosis-related | Hs.3280 |
| | | | | | | | cysteine protease | |
| 208677_s_at | 466 | 2.34 | 1.72E−08 | 47.26% | 31.21% | BSG | basigin (OK blood group) | Hs.74631 |
| 203744_at | 306 | 2.34 | 2.96E−13 | 31.01% | 19.36% | HMG4 | high-mobility group | Hs.19114 |
| | | | | | | | (nonhistone chromosomal) | |
| | | | | | | | protein 4 | |
| 212358_at | 596 | 2.34 | 2.49E−02 | 146.05% | 33.71% | CLIPR-59 | CLIP-170-related protein | Hs.7357 |
| 201036_s_at | 187 | 2.33 | 1.53E−05 | 68.07% | 19.36% | HADHSC | L-3-hydroxyacyl-Coenzyme A | Hs.8110 |
| | | | | | | | dehydrogenase, short chain | |
| 205600_x_at | 404 | 2.33 | 1.45E−07 | 51.99% | 32.81% | HOXB5 | homeo box B5 | Hs.22554 |
| 219007_at | 750 | 2.31 | 1.48E−05 | 67.23% | 30.35% | FLJ13287 | hypothetical protein FLJ13287 | Hs.53263 |
| 201069_at | 190 | 2.31 | 3.71E−03 | 109.02% | 24.70% | MMP2 | matrix metalloproteinase 2 | Hs.111301 |
| | | | | | | | (gelatinase A, 72 kD | |
| | | | | | | | gelatinase, 72 kD type IV | |
| | | | | | | | collagenase) | |
| 201231_s_at | 195 | 2.30 | 5.73E−10 | 40.37% | 18.11% | ENO1 | enolase 1, (alpha) | Hs.254105 |
| 218409_s_at | 724 | 2.29 | 1.56E−03 | 98.22% | 22.49% | DNAJL1 | hypothetical protein similar to | Hs.13015 |
| | | | | | | | mouse Dnajl1 | |
| 221471_at | 795 | 2.29 | 1.27E−08 | 45.85% | 23.06% | TDE1 | tumor differentially expressed 1 | Hs.272168 |
| 216705_s_at | 685 | 2.28 | 8.43E−07 | 56.23% | 28.91% | ADA | adenosine deaminase | Hs.1217 |
| 205601_s_at | 405 | 2.28 | 3.00E−05 | 70.06% | 24.09% | HOXB5 | homeo box B5 | Hs.22554 |
| 209208_at | 489 | 2.28 | 3.02E−07 | 53.16% | 28.79% | MPDU1 | mannose-P-dolichol utilization | Hs.6710 |
| | | | | | | | defect 1 | |
| 218188_s_at | 716 | 2.27 | 2.80E−08 | 47.33% | 21.04% | TIMM13 | translocase of inner | Hs.23410 |
| | | | | | | | mitochondrial membrane 13 | |
| | | | | | | | homolog (yeast) | |
| 200983_x_at | 182 | 2.27 | 8.67E−06 | 64.32% | 25.73% | CD59 | CD59 antigen p18-20 (antigen | Hs.278573 |
| | | | | | | | identified by monoclonal | |
| | | | | | | | antibodies 16.3A5, EJ16, | |
| | | | | | | | EJ30, EL32 and G344) | |
| 208964_s_at | 480 | 2.27 | 3.72E−10 | 39.28% | 19.16% | FADS1 | fatty acid desaturase 1 | Hs.132898 |
| 217274_x_at | 690 | 2.27 | 2.17E−03 | 99.73% | 56.76% | MYL4 | myosin, light polypeptide 4, | Hs.433562 |
| | | | | | | | alkali; atrial, embryonic | |
| 210365_at | 534 | 2.27 | 1.71E−05 | 66.55% | 41.85% | RUNX1 | runt-related transcription factor | Hs.129914 |
| | | | | | | | 1 (acute myeloid leukemia 1; | |
| | | | | | | | aml1 oncogene) | |
| 214455_at | 653 | 2.27 | 2.04E−03 | 100.36% | 21.81% | H2BFA, | H2B histone family, member | Hs.356901 |
| | | | | | | H2BFL | A, H2B histone family, | |
| | | | | | | | member L | |
| 220741_s_at | 786 | 2.27 | 1.33E−06 | 57.27% | 31.33% | SID6-306 | inorganic pyrophosphatase | Hs.375016 |
| 218585_s_at | 728 | 2.25 | 6.54E−04 | 88.37% | 35.75% | RAMP | RA-regulated nuclear matrix- | Hs.126774 |
| | | | | | | | associated protein | |
| 205608_s_at | 406 | 2.25 | 3.35E−08 | 47.27% | 23.20% | ANGPT1 | angiopoietin 1 | Hs.2463 |
| 205453_at | 397 | 2.24 | 9.34E−05 | 74.65% | 34.31% | HOXB2 | homeo box B2 | Hs.2733 |
| 201890_at | 228 | 2.24 | 5.28E−03 | 111.27% | 22.47% | RRM2 | ribonucleotide reductase M2 | Hs.75319 |
| | | | | | | | polypeptide | |
| 204386_s_at | 342 | 2.23 | 2.36E−07 | 51.76% | 22.35% | MRP63 | mitochondrial ribosomal | Hs.182695 |
| | | | | | | | protein 63 | |
| 210052_s_at | 523 | 2.23 | 9.78E−07 | 55.82% | 20.14% | C20orf1 | chromosome 20 open reading | Hs.9329 |
| | | | | | | | frame 1 | |
| 208898_at | 477 | 2.23 | 1.62E−07 | 50.69% | 23.80% | ATP6V1D | ATPase, H+ transporting, | Hs.272630 |
| | | | | | | | lysosomal 34 kD, V1 subunit D | |
| 200821_at | 170 | 2.22 | 5.72E−08 | 47.87% | 26.92% | LAMP2 | lysosomal-associated | Hs.8262 |
| | | | | | | | membrane protein 2 | |
| 207719_x_at | 455 | 2.21 | 2.09E−13 | 29.62% | 22.01% | KIAA0470 | KIAA0470 gene product | Hs.25132 |
| 204438_at | 21 | 2.21 | 2.04E−03 | 98.49% | 17.08% | MRC1 | mannose receptor, C type 1 | Hs.75182 |
| 209199_s_at | 486 | 2.21 | 5.25E−05 | 70.69% | 35.75% | MEF2C | MADS box transcription | Hs.78995 |
| | | | | | | | enhancer factor 2, polypeptide | |
| | | | | | | | C (myocyte enhancer factor | |
| | | | | | | | 2C) | |
| 214500_at | 655 | 2.21 | 5.45E−04 | 85.81% | 30.19% | H2AFY | H2A histone family, member Y | Hs.75258 |
| 201028_s_at | 186 | 2.21 | 3.32E−06 | 59.25% | 21.39% | MIC2 | antigen identified by | Hs.433387 |
| | | | | | | | monoclonal antibodies 12E7, | |
| | | | | | | | F21 and O13 | |
| 209395_at | 495 | 2.21 | 3.51E−02 | 148.36% | 52.07% | CHI3L1 | chitinase 3-like 1 (cartilage | Hs.75184 |
| | | | | | | | glycoprotein-39) | |
| 216554_s_at | 683 | 2.20 | 5.42E−13 | 30.22% | 18.05% | ENO1 | enolase 1, (alpha) | Hs.381397 |
| 222294_s_at | 812 | 2.20 | 2.12E−04 | 78.67% | 31.23% | | | Hs.432533 |
| 203688_at | 303 | 2.20 | 3.64E−06 | 59.34% | 25.67% | PKD2 | polycystic kidney disease 2 | Hs.82001 |
| | | | | | | | (autosomal dominant) | |
| 200728_at | 163 | 2.20 | 2.37E−12 | 32.00% | 25.79% | ACTR2 | ARP2 actin-related protein 2 | Hs.396278 |
| | | | | | | | homolog (yeast) | |
| 201562_s_at | 216 | 2.20 | 1.75E−14 | 27.69% | 29.44% | SORD | sorbitol dehydrogenase | Hs.878 |
| 211714_x_at | 567 | 2.19 | 5.66E−07 | 53.34% | 16.95% | FKBP1A | FK506 binding protein 1A | Hs.179661 |
| | | | | | | | (12 kD) | |
| 206057_x_at | 422 | 2.19 | 7.42E−12 | 33.11% | 25.12% | SPN | sialophorin (gpL115, | Hs.80738 |
| | | | | | | | leukosialin, CD43) | |
| 207761_s_at | 457 | 2.19 | 8.33E−06 | 62.25% | 19.69% | DKFZP586A0522 | DKFZP586A0522 protein | Hs.288771 |
| 200769_s_at | 165 | 2.18 | 1.09E−07 | 48.80% | 26.93% | MAT2A | methionine | Hs.77502 |
| | | | | | | | adenosyltransferase II, alpha | |
| 206665_s_at | 439 | 2.18 | 4.65E−03 | 106.39% | 44.14% | BCL2L1 | BCL2-like 1 | Hs.305890 |
| 208858_s_at | 473 | 2.17 | 2.26E−07 | 50.14% | 37.12% | KIAA0747 | KIAA0747 protein | Hs.8309 |
| 205239_at | 386 | 2.17 | 3.39E−02 | 144.04% | 72.62% | AREG | amphiregulin (schwannoma- | Hs.270833 |
| | | | | | | | derived growth factor) | |
| 205919_at | 419 | 2.17 | 4.72E−03 | 105.44% | 54.93% | HBE1 | hemoglobin, epsilon 1 | Hs.117848 |
| 203253_s_at | 288 | 2.17 | 1.36E−08 | 44.04% | 22.47% | KIAA0433 | KIAA0433 protein | Hs.26179 |
| 210549_s_at | 542 | 2.17 | 8.57E−04 | 88.61% | 0.00% | SCYA23 | small inducible cytokine | Hs.169191 |
| | | | | | | | subfamily A (Cys-Cys), | |
| | | | | | | | member 23 | |
| 201329_s_at | 203 | 2.16 | 5.35E−04 | 82.28% | 57.70% | ETS2 | v-ets erythroblastosis virus | Hs.85146 |
| | | | | | | | E26 oncogene homolog 2 | |
| | | | | | | | (avian) | |
| 204429_s_at | 348 | 2.16 | 1.40E−05 | 63.30% | 28.97% | SLC2A5 | solute carrier family 2 | Hs.33084 |
| | | | | | | | (facilitated glucose/fructose | |
| | | | | | | | transporter), member 5 | |
| 218136_s_at | 713 | 2.15 | 3.01E−02 | 137.41% | 93.36% | LOC51312 | mitochondrial solute carrier | Hs.283716 |
| 200806_s_at | 168 | 2.15 | 1.71E−06 | 55.72% | 20.60% | HSPD1 | heat shock 60 kD protein 1 | Hs.79037 |
| | | | | | | | (chaperonin) | |
| 212296_at | 594 | 2.15 | 9.97E−09 | 43.04% | 17.60% | POH1 | 26S proteasome-associated | Hs.178761 |
| | | | | | | | pad1 homolog | |
| 218160_at | 715 | 2.14 | 4.05E−06 | 58.42% | 24.57% | NDUFA8 | NADH dehydrogenase | Hs.31547 |
| | | | | | | | (ubiquinone) 1 alpha | |
| | | | | | | | subcomplex, 8 (19 kD, PGIV) | |
| 204039_at | 323 | 2.14 | 7.35E−04 | 85.48% | 36.46% | CEBPA | CCAAT/enhancer binding | Hs.76171 |
| | | | | | | | protein (C/EBP), alpha | |
| 200727_s_at | 162 | 2.14 | 4.97E−11 | 34.77% | 36.28% | ACTR2 | ARP2 actin-related protein 2 | Hs.393201 |
| | | | | | | | homolog (yeast) | |
| 48808_at | 823 | 2.13 | 4.23E−02 | 151.12% | 14.58% | DHFR | dihydrofolate reductase | Hs.83765 |
| 222037_at | 808 | 2.13 | 3.35E−04 | 79.27% | 35.71% | MCM4 | MCM4 minichromosome | Hs.319215 |
| | | | | | | | maintenance deficient 4 (S. cerevisiae) | |
| 202345_s_at | 248 | 2.13 | 8.72E−04 | 86.92% | 27.92% | FABP5 | fatty acid binding protein 5 | Hs.153179 |
| | | | | | | | (psoriasis-associated) | |
| 210036_s_at | 521 | 2.12 | 1.28E−03 | 90.00% | 31.48% | KCNH2 | potassium voltage-gated | Hs.188021 |
| | | | | | | | channel, subfamily H (eag- | |
| | | | | | | | related), member 2 | |
| 200812_at | 169 | 2.12 | 1.07E−05 | 61.36% | 26.73% | CCT7 | chaperonin containing TCP1, | Hs.108809 |
| | | | | | | | subunit 7 (eta) | |
| 202974_at | 277 | 2.12 | 2.27E−04 | 75.68% | 43.58% | MPP1 | membrane protein, | Hs.1861 |
| | | | | | | | palmitoylated 1 (55 kD) | |
| 201577_at | 221 | 2.11 | 1.31E−07 | 47.86% | 22.32% | NME1 | non-metastatic cells 1, protein | Hs.118638 |
| | | | | | | | (NM23A) expressed in | |
| 202201_at | 241 | 2.11 | 1.87E−03 | 92.07% | 49.52% | BLVRB | biliverdin reductase B (flavin | Hs.76289 |
| | | | | | | | reductase (NADPH)) | |
| 210849_s_at | 552 | 2.11 | 1.31E−10 | 35.54% | 31.11% | VPS41 | vacuolar protein sorting 41 | Hs.180941 |
| | | | | | | | (yeast) | |
| 209365_s_at | 494 | 2.10 | 3.90E−06 | 56.91% | 34.40% | ECM1 | extracellular matrix protein 1 | Hs.81071 |
| 217988_at | 705 | 2.10 | 8.48E−06 | 60.04% | 23.33% | HEI10 | enhancer of invasion 10 | Hs.107003 |
| 203904_x_at | 313 | 2.10 | 4.53E−08 | 45.10% | 27.01% | KAI1 | kangai 1 (suppression of | Hs.323949 |
| | | | | | | | tumorigenicity 6, prostate; | |
| | | | | | | | CD82 antigen (R2 leukocyte | |
| | | | | | | | antigen, antigen detected by | |
| | | | | | | | monoclonal and antibody IA4)) | |
| 200986_at | 35 | 2.09 | 1.08E−04 | 71.48% | 22.84% | SERPING1 | serine (or cysteine) proteinase | Hs.151242 |
| | | | | | | | inhibitor, clade G (C1 | |
| | | | | | | | inhibitor), member 1, | |
| | | | | | | | (angioedema, hereditary) | |
| 201491_at | 211 | 2.09 | 7.56E−06 | 59.51% | 18.40% | C14orf3 | chromosome 14 open reading | Hs.204041 |
| | | | | | | | frame 3 | |
| 200942_s_at | 178 | 2.09 | 1.47E−08 | 42.77% | 22.51% | HSBP1 | heat shock factor binding | Hs.250899 |
| | | | | | | | protein 1 | |
| 200973_s_at | 181 | 2.09 | 8.67E−08 | 46.27% | 30.93% | TSPAN-3 | tetraspan 3 | Hs.100090 |
| 207943_x_at | 459 | 2.09 | 2.78E−09 | 39.76% | 25.61% | PLAGL1 | pleiomorphic adenoma gene- | Hs.75825 |
| | | | | | | | like 1 | |
| 208899_x_at | 478 | 2.09 | 3.61E−09 | 40.15% | 27.32% | ATP6V1D | ATPase, H+ transporting, | Hs.272630 |
| | | | | | | | lysosomal 34 kD, V1 subunit D | |
| 204187_at | 334 | 2.09 | 3.03E−02 | 133.16% | 94.60% | GMPR | guanosine monophosphate | Hs.1435 |
| | | | | | | | reductase | |
| 220240_s_at | 777 | 2.08 | 2.48E−07 | 48.85% | 18.46% | FLJ20623 | hypothetical protein FLJ20623 | Hs.27337 |
| 218966_at | 749 | 2.08 | 3.83E−05 | 65.76% | 27.14% | MYO5C | myosin 5C | Hs.111782 |
| 214321_at | 649 | 2.07 | 4.28E−02 | 146.79% | 35.71% | NOV | nephroblastoma | Hs.235935 |
| | | | | | | | overexpressed gene | |
| 211769_x_at | 570 | 2.07 | 2.26E−09 | 39.09% | 24.73% | TDE1 | tumor differentially expressed 1 | Hs.272168 |
| 202990_at | 279 | 2.07 | 1.72E−04 | 73.21% | 26.24% | PYGL | phosphorylase, glycogen; liver | Hs.771 |
| | | | | | | | (Hers disease, glycogen | |
| | | | | | | | storage disease type VI) | |
| 202429_s_at | 251 | 2.06 | 5.39E−06 | 57.32% | 26.50% | PPP3CA | protein phosphatase 3 | Hs.272458 |
| | | | | | | | (formerly 2B), catalytic subunit, | |
| | | | | | | | alpha isoform (calcineurin A | |
| | | | | | | | alpha) | |
| 209215_at | 490 | 2.06 | 2.44E−05 | 62.66% | 37.86% | TETRAN | tetracycline transporter-like | Hs.157145 |
| | | | | | | | protein | |
| 217949_s_at | 703 | 2.06 | 9.23E−06 | 59.41% | 20.57% | IMAGE3455200 | hypothetical protein | Hs.324844 |
| | | | | | | | IMAGE3455200 | |
| 205330_at | 392 | 2.06 | 9.95E−03 | 112.06% | 45.65% | MN1 | meningioma (disrupted in | Hs.268515 |
| | | | | | | | balanced translocation) 1 | |
| 218027_at | 708 | 2.06 | 7.08E−08 | 45.38% | 19.16% | MRPL15 | mitochondrial ribosomal | Hs.18349 |
| | | | | | | | protein L15 | |
| 219479_at | 761 | 2.06 | 6.63E−04 | 82.11% | 23.65% | MGC5302 | endoplasmic reticulum | Hs.44970 |
| | | | | | | | resident protein 58; | |
| | | | | | | | hypothetical protein MGC5302 | |
| 215416_s_at | 671 | 2.06 | 1.08E−10 | 34.37% | 18.21% | STOML2 | stomatin (EPB72)-like 2 | Hs.3439 |
| 221479_s_at | 796 | 2.06 | 9.03E−03 | 110.65% | 34.64% | BNIP3L | BCL2/adenovirus E1B 19 kD | Hs.132955 |
| | | | | | | | interacting protein 3-like | |
| 215285_s_at | 669 | 2.05 | 1.83E−03 | 90.98% | 18.13% | PHTF1 | putative homeodomain | Hs.123637 |
| | | | | | | | transcription factor 1 | |
| 219559_at | 763 | 2.05 | 9.10E−10 | 37.29% | 24.99% | C20orf59 | chromosome 20 open reading | Hs.353013 |
| | | | | | | | frame 59 | |
| 211342_x_at | 557 | 2.05 | 4.07E−08 | 42.42% | 51.95% | TNRC11 | trinucleotide repeat containing | Hs.211607 |
| | | | | | | | 11 (THR-associated protein, | |
| | | | | | | | 230 kD subunit) | |
| 210298_x_at | 71 | 2.05 | 4.94E−03 | 101.70% | 26.72% | FHL1 | four and a half LIM domains 1 | Hs.239069 |
| 217724_at | 694 | 2.04 | 6.51E−07 | 50.51% | 16.73% | PAI-RBP1 | PAI-1 mRNA-binding protein | Hs.165998 |
| 208817_at | 471 | 2.04 | 1.23E−08 | 41.49% | 24.81% | COMT | catechol-O-methyltransferase | Hs.240013 |
| 204040_at | 324 | 2.04 | 1.37E−05 | 60.01% | 30.27% | KIAA0161 | KIAA0161 gene product | Hs.78894 |
| 213854_at | 639 | 2.04 | 4.56E−07 | 49.43% | 20.27% | SYNGR1 | synaptogyrin 1 | Hs.6139 |
| 200729_s_at | 164 | 2.04 | 1.28E−11 | 31.75% | 24.98% | ACTR2 | ARP2 actin-related protein 2 | Hs.393201 |
| | | | | | | | homolog (yeast) | |
| 201970_s_at | 232 | 2.04 | 3.64E−04 | 76.63% | 31.58% | NASP | nuclear autoantigenic sperm | Hs.380400 |
| | | | | | | | protein (histone-binding) | |
| 203021_at | 280 | 2.03 | 3.92E−04 | 76.95% | 33.19% | SLPI | secretory leukocyte protease | Hs.251754 |
| | | | | | | | inhibitor (antileukoproteinase) | |
| 200900_s_at | 175 | 2.03 | 8.48E−06 | 58.01% | 25.64% | M6PR | mannose-6-phosphate | Hs.134084 |
| | | | | | | | receptor (cation dependent) | |
| 203800_s_at | 308 | 2.03 | 7.24E−07 | 50.35% | 21.68% | MRPS14 | mitochondrial ribosomal | Hs.247324 |
| | | | | | | | protein S14 | |
| 212320_at | 595 | 2.02 | 2.59E−07 | 47.68% | 15.36% | | | Hs.179661 |
| 217892_s_at | 701 | 2.02 | 1.64E−10 | 34.53% | 25.93% | ARL4, | ADP-ribosylation factor-like 4, | Hs.10706 |
| | | | | | | EPLIN | epithelial protein lost in | |
| | | | | | | | neoplasm beta | |
| 218270_at | 719 | 2.02 | 2.16E−05 | 61.02% | 34.29% | MRPL24 | mitochondrial ribosomal | Hs.9265 |
| | | | | | | | protein L24 | |
| 201302_at | 199 | 2.02 | 1.45E−05 | 59.43% | 31.19% | ANXA4 | annexin A4 | Hs.77840 |
| 214113_s_at | 61 | 2.02 | 4.98E−06 | 56.07% | 12.21% | RBM8A | RNA binding motif protein 8A | Hs.10283 |
| 206438_x_at | 434 | 2.01 | 2.03E−11 | 31.90% | 26.02% | FLJ12975 | hypothetical protein FLJ12975 | Hs.167165 |
| 205505_at | 399 | 2.01 | 1.77E−05 | 60.46% | 21.22% | GCNT1 | glucosaminyl (N-acetyl) | Hs.159642 |
| | | | | | | | transferase 1, core 2 (beta- | |
| | | | | | | | 1,6-N- | |
| | | | | | | | acetylglucosaminyltransferase) | |
| 209515_s_at | 499 | 2.01 | 6.79E−05 | 66.13% | 27.14% | RAB27A | RAB27A, member RAS | Hs.50477 |
| | | | | | | | oncogene family | |
| 221831_at | 802 | 2.01 | 1.72E−04 | 69.36% | 52.04% | | | Hs.348515 |
| 221942_s_at | 806 | 2.01 | 1.14E−07 | 44.95% | 33.24% | GUCY1A3 | guanylate cyclase 1, soluble, | Hs.75295 |
| | | | | | | | alpha 3 | |
| 213797_at | 101 | 2.01 | 4.76E−04 | 77.51% | 26.86% | cig5 | vipirin | Hs.17518 |
| 209517_s_at | 500 | 2.00 | 4.18E−09 | 38.85% | 19.12% | ASH2L | ash2 (absent, small, or | Hs.6856 |
| | | | | | | | homeotic)-like (Drosophila) | |
| 213617_s_at | 634 | 2.00 | 2.38E−09 | 37.89% | 23.87% | DKFZP586M1523 | DKFZP586M1523 protein | Hs.22981 |
| 214390_s_at | 650 | 2.00 | 1.54E−02 | 116.91% | 34.44% | BCAT1 | branched chain | Hs.317432 |
| | | | | | | | aminotransferase 1, cytosolic | |
| 219423_x_at | 760 | 0.50 | 8.47E−11 | 61.84% | 27.11% | TNFRSF12 | tumor necrosis factor receptor | Hs.180338 |
| | | | | | | | superfamily, member 12 | |
| | | | | | | | (translocating chain- | |
| | | | | | | | association membrane protein) | |
| 35626_at | 816 | 0.50 | 1.86E−06 | 91.46% | 39.11% | SGSH | N-sulfoglucosamine | Hs.31074 |
| | | | | | | | sulfohydrolase (sulfamidase) | |
| 211984_at | 581 | 0.50 | 2.35E−15 | 48.17% | 17.35% | | | Hs.374441 |
| 200965_s_at | 180 | 0.50 | 6.00E−07 | 96.72% | 24.80% | ABLIM | actin binding LIM protein | Hs.158203 |
| 201531_at | 214 | 0.50 | 7.92E−11 | 59.64% | 30.26% | ZFP36 | zinc finger protein 36, C3H | Hs.343586 |
| | | | | | | | type, homolog (mouse) | |
| 205022_s_at | 379 | 0.49 | 3.82E−12 | 26.84% | 36.11% | CHES1 | checkpoint suppressor 1 | Hs.211773 |
| 207697_x_at | 454 | 0.49 | 3.04E−09 | 78.11% | 19.85% | LILRB1, | leukocyte immunoglobulin-like | Hs.22405 |
| | | | | | | LILRB2 | receptor, subfamily B (with TM | |
| | | | | | | | and ITIM domains), member 1, | |
| | | | | | | | leukocyte immunoglobulin-like | |
| | | | | | | | receptor, subfamily B (with TM | |
| | | | | | | | and ITIM domains), member 2 | |
| 205019_s_at | 378 | 0.49 | 1.92E−10 | 62.69% | 30.88% | VIPR1 | vasoactive intestinal peptide | Hs.348500 |
| | | | | | | | receptor 1 | |
| 210845_s_at | 551 | 0.49 | 1.37E−07 | 66.07% | 46.38% | PLAUR | plasminogen activator, | Hs.179657 |
| | | | | | | | urokinase receptor | |
| 213831_at | 637 | 0.49 | 1.63E−03 | 90.56% | 91.29% | HLA-DQA1 | major histocompatibility | Hs.198253 |
| | | | | | | | complex, class II, DQ alpha 1 | |
| 203341_at | 292 | 0.49 | 6.80E−17 | 34.29% | 25.70% | CBF2 | CCAAT-box-binding | Hs.184760 |
| | | | | | | | transcription factor | |
| 209657_s_at | 506 | 0.49 | 6.13E−14 | 51.61% | 24.06% | HSF2 | heat shock transcription factor 2 | Hs.158195 |
| 220684_at | 784 | 0.49 | 7.01E−09 | 71.86% | 34.98% | TBX21 | T-box 21 | Hs.272409 |
| 211924_s_at | 577 | 0.49 | 4.60E−05 | 82.81% | 65.29% | PLAUR | plasminogen activator, | Hs.179657 |
| | | | | | | | urokinase receptor | |
| 32032_at | 815 | 0.49 | 5.45E−18 | 33.09% | 24.48% | DGSI | DiGeorge syndrome critical | Hs.154879 |
| | | | | | | | region gene DGSI; likely | |
| | | | | | | | ortholog of mouse expressed | |
| | | | | | | | sequence 2 embryonic lethal | |
| 212914_at | 610 | 0.49 | 6.70E−09 | 76.90% | 30.67% | PKP4 | plakophilin 4 | Hs.356416 |
| 204847_at | 370 | 0.49 | 2.64E−20 | 37.08% | 18.34% | ZNF- | zinc finger protein | Hs.301956 |
| | | | | | | U69274 | | |
| 218559_s_at | 727 | 0.49 | 3.58E−03 | 191.41% | 42.94% | MAFB | v-maf musculoaponeurotic | Hs.169487 |
| | | | | | | | fibrosarcoma oncogene | |
| | | | | | | | homolog B (avian) | |
| 213587_s_at | 633 | 0.49 | 5.00E−10 | 60.46% | 35.98% | | | Hs.351612 |
| 203547_at | 297 | 0.48 | 8.38E−13 | 57.70% | 24.56% | CD4 | CD4 antigen (p55) | Hs.17483 |
| 214696_at | 662 | 0.48 | 1.43E−08 | 82.10% | 29.38% | MGC14376 | hypothetical protein | Hs.417157 |
| | | | | | | | MGC14376 | |
| 220088_at | 775 | 0.48 | 1.73E−04 | 116.92% | 60.98% | C5R1 | complement component 5 | Hs.2161 |
| | | | | | | | receptor 1 (C5a ligand) | |
| 202724_s_at | 262 | 0.48 | 5.23E−11 | 63.15% | 29.60% | FOXO1A | forkhead box O1A | Hs.170133 |
| | | | | | | | (rhabdomyosarcoma) | |
| 200788_s_at | 166 | 0.48 | 1.43E−12 | 61.50% | 19.94% | PEA15 | phosphoprotein enriched in | Hs.194673 |
| | | | | | | | astrocytes 15 | |
| 213376_at | 626 | 0.48 | 1.04E−14 | 49.81% | 24.43% | | | Hs.372699 |
| 204621_s_at | 357 | 0.48 | 1.11E−08 | 79.04% | 32.70% | NR4A2 | nuclear receptor subfamily 4, | Hs.82120 |
| | | | | | | | group A, member 2 | |
| 214945_at | 664 | 0.48 | 3.42E−07 | 63.69% | 51.89% | KIAA0752 | KIAA0752 protein | Hs.126779 |
| 221757_at | 801 | 0.48 | 5.42E−11 | 69.15% | 23.27% | MGC17330 | hypothetical protein | Hs.26670 |
| | | | | | | | MGC17330 | |
| 211985_s_at | 582 | 0.48 | 3.30E−12 | 62.39% | 23.79% | | | Hs.374441 |
| 200871_s_at | 174 | 0.48 | 1.63E−09 | 81.31% | 16.45% | PSAP | prosaposin (variant Gaucher | Hs.406455 |
| | | | | | | | disease and variant | |
| | | | | | | | metachromatic | |
| | | | | | | | leukodystrophy) | |
| 202842_s_at | 267 | 0.48 | 2.16E−14 | 52.79% | 23.79% | DNAJB9 | DnaJ (Hsp40) homolog, | Hs.6790 |
| | | | | | | | subfamily B, member 9 | |
| 219155_at | 756 | 0.48 | 8.61E−16 | 47.62% | 23.40% | RDGBB | retinal degeneration B beta | Hs.333212 |
| 203234_at | 287 | 0.48 | 2.03E−07 | 89.59% | 37.67% | UP | uridine phosphorylase | Hs.77573 |
| 219040_at | 752 | 0.48 | 6.47E−10 | 42.85% | 43.00% | FLJ22021 | hypothetical protein FLJ22021 | Hs.7258 |
| 214714_at | 663 | 0.48 | 2.31E−17 | 47.52% | 14.02% | FLJ12298 | hypothetical protein FLJ12298 | Hs.284168 |
| 219279_at | 758 | 0.47 | 4.42E−11 | 68.97% | 25.55% | FLJ20220 | hypothetical protein FLJ20220 | Hs.21126 |
| 40420_at | 822 | 0.47 | 4.30E−19 | 39.97% | 20.91% | STK10 | serine/threonine kinase 10 | Hs.16134 |
| 214467_at | 96 | 0.47 | 8.57E−09 | 86.65% | 24.10% | GPR65 | G protein-coupled receptor 65 | Hs.131924 |
| 202518_at | 256 | 0.47 | 4.27E−19 | 42.88% | 17.86% | BCL7B | B-cell CLL/lymphoma 7B | Hs.16269 |
| 204224_s_at | 338 | 0.47 | 4.35E−15 | 53.97% | 19.72% | GCH1 | GTP cyclohydrolase 1 (dopa- | Hs.86724 |
| | | | | | | | responsive dystonia) | |
| 203045_at | 281 | 0.47 | 3.33E−07 | 92.08% | 40.13% | NINJ1 | ninjurin 1 | Hs.11342 |
| 39582_at | 821 | 0.47 | 1.97E−11 | 70.10% | 20.79% | | | Hs.26295 |
| 210225_x_at | 529 | 0.47 | 3.53E−07 | 98.45% | 34.82% | LILRB3 | leukocyte immunoglobulin-like | Hs.105928 |
| | | | | | | | receptor, subfamily B (with TM | |
| | | | | | | | and ITIM domains), member 3 | |
| 204891_s_at | 374 | 0.47 | 5.17E−05 | 128.95% | 45.60% | LCK | lymphocyte-specific protein | Hs.1765 |
| | | | | | | | tyrosine kinase | |
| 218711_s_at | 733 | 0.47 | 1.60E−12 | 34.72% | 36.28% | SDPR | serum deprivation response | Hs.26530 |
| | | | | | | | (phosphatidylserine binding | |
| | | | | | | | protein) | |
| 205254_x_at | 388 | 0.47 | 4.07E−07 | 104.29% | 28.42% | TCF7 | transcription factor 7 (T-cell | Hs.169294 |
| | | | | | | | specific, HMG-box) | |
| 204396_s_at | 344 | 0.47 | 4.98E−11 | 72.12% | 23.82% | GPRK5 | G protein-coupled receptor | Hs.211569 |
| | | | | | | | kinase 5 | |
| 204369_at | 341 | 0.47 | 1.47E−14 | 47.33% | 28.81% | PIK3CA | phosphoinositide-3-kinase, | Hs.85701 |
| | | | | | | | catalytic, alpha polypeptide | |
| 212998_x_at | 611 | 0.47 | 3.46E−09 | 72.57% | 38.15% | HLA-DQB1 | major histocompatibility | Hs.73931 |
| | | | | | | | complex, class II, DQ beta 1 | |
| 204588_s_at | 354 | 0.47 | 1.36E−06 | 111.56% | 31.06% | SLC7A7 | solute carrier family 7 (cationic | Hs.194693 |
| | | | | | | | amino acid transporter, y+ | |
| | | | | | | | system), member 7 | |
| 208881_x_at | 475 | 0.47 | 2.85E−21 | 33.87% | 21.20% | IDI1 | isopentenyl-diphosphate delta | Hs.76038 |
| | | | | | | | isomerase | |
| 202861_at | 270 | 0.47 | 1.34E−08 | 76.10% | 40.36% | PER1 | period homolog 1 (Drosophila) | Hs.68398 |
| 218828_at | 739 | 0.46 | 5.31E−06 | 70.98% | 62.75% | PLSCR3 | phospholipid scramblase 3 | Hs.103382 |
| 202388_at | 250 | 0.46 | 2.71E−11 | 71.26% | 25.16% | RGS2 | regulator of G-protein | Hs.78944 |
| | | | | | | | signalling 2, 24 kD | |
| 219118_at | 755 | 0.46 | 4.33E−09 | 60.48% | 44.50% | FKBP11 | FK506 binding protein 11 (19 kDa) | Hs.24048 |
| 213906_at | 640 | 0.46 | 2.86E−06 | 109.54% | 42.47% | MYBL1 | v-myb myeloblastosis viral | Hs.300592 |
| | | | | | | | oncogene homolog (avian)-like 1 | |
| 202880_s_at | 273 | 0.46 | 9.28E−17 | 51.09% | 19.25% | PSCD1 | pleckstrin homology, Sec7 and | Hs.1050 |
| | | | | | | | coiled/coil domains | |
| | | | | | | | 1(cytohesin 1) | |
| 201631_s_at | 223 | 0.46 | 2.35E−04 | 129.87% | 65.59% | IER3 | immediate early response 3 | Hs.76095 |
| 213758_at | 635 | 0.46 | 1.89E−14 | 53.82% | 26.63% | | | Hs.373513 |
| 209616_s_at | 505 | 0.46 | 1.05E−06 | 93.94% | 48.20% | CES1 | carboxylesterase 1 | Hs.76688 |
| | | | | | | | (monocyte/macrophage serine | |
| | | | | | | | esterase 1) | |
| 205281_s_at | 390 | 0.46 | 1.44E−16 | 51.93% | 20.24% | PIGA | phosphatidylinositol glycan, | Hs.51 |
| | | | | | | | class A (paroxysmal nocturnal | |
| | | | | | | | hemoglobinuria) | |
| 204215_at | 337 | 0.46 | 1.33E−13 | 57.29% | 27.83% | MGC4175 | hypothetical protein MGC4175 | Hs.322404 |
| 212812_at | 98 | 0.46 | 6.01E−10 | 72.92% | 35.84% | | | Hs.288232 |
| 207826_s_at | 458 | 0.45 | 2.92E−06 | 63.43% | 63.90% | ID3 | inhibitor of DNA binding 3, | Hs.76884 |
| | | | | | | | dominant negative helix-loop- | |
| | | | | | | | helix protein | |
| 202072_at | 237 | 0.45 | 5.57E−04 | 111.63% | 84.78% | HNRPL | heterogeneous nuclear | Hs.2730 |
| | | | | | | | ribonucleoprotein L | |
| 210439_at | 538 | 0.45 | 2.90E−06 | 112.93% | 44.33% | ICOS | inducible T-cell co-stimulator | Hs.56247 |
| 203320_at | 290 | 0.45 | 3.65E−15 | 55.50% | 24.57% | LNK | lymphocyte adaptor protein | Hs.13131 |
| 204440_at | 349 | 0.45 | 1.79E−10 | 68.74% | 36.26% | CD83 | CD83 antigen (activated B | Hs.79197 |
| | | | | | | | lymphocytes, immunoglobulin | |
| | | | | | | | superfamily) | |
| 211458_s_at | 559 | 0.45 | 1.95E−10 | 69.84% | 35.88% | GABARAPL3 | GABA(A) receptors associated | Hs.334497 |
| | | | | | | | protein like 3 | |
| 212769_at | 608 | 0.45 | 1.48E−10 | 56.88% | 40.54% | TLE3 | transducin-like enhancer of | Hs.287362 |
| | | | | | | | split 3 (E(sp1) homolog, | |
| | | | | | | | Drosophila) | |
| 221841_s_at | 803 | 0.45 | 9.97E−06 | 134.32% | 33.96% | KLF4 | Kruppel-like factor 4 (gut) | Hs.376206 |
| 217784_at | 696 | 0.45 | 1.90E−12 | 60.94% | 31.98% | YKT6 | SNARE protein Ykt6 | Hs.296244 |
| 202782_s_at | 265 | 0.45 | 2.24E−14 | 51.88% | 30.16% | SKIP | skeletal muscle and kidney | Hs.178347 |
| | | | | | | | enriched inositol phosphatase | |
| 220987_s_at | 94 | 0.45 | 9.43E−16 | 56.70% | 21.86% | DKFZP434J037 | hypothetical protein | Hs.172012 |
| | | | | | | | DKFZp434J037 | |
| 218708_at | 732 | 0.45 | 2.34E−14 | 39.15% | 33.34% | NXT1 | NTF2-like export factor 1 | Hs.24563 |
| 215785_s_at | 674 | 0.45 | 6.95E−10 | 68.97% | 40.16% | CYFIP2 | cytoplasmic FMR1 interacting | Hs.258503 |
| | | | | | | | protein 2 | |
| 202969_at | 276 | 0.45 | 2.29E−16 | 49.47% | 26.00% | | | Hs.432856 |
| 207000_s_at | 445 | 0.45 | 1.12E−13 | 66.37% | 20.02% | PPP3CC | protein phosphatase 3 | Hs.75206 |
| | | | | | | | (formerly 2B), catalytic subunit, | |
| | | | | | | | gamma isoform (calcineurin A | |
| | | | | | | | gamma) | |
| 203555_at | 298 | 0.45 | 2.68E−15 | 46.47% | 29.83% | PTPN18 | protein tyrosine phosphatase, | Hs.278597 |
| | | | | | | | non-receptor type 18 (brain- | |
| | | | | | | | derived) | |
| 202928_s_at | 274 | 0.45 | 6.61E−13 | 54.32% | 33.85% | PHF1 | PHD finger protein 1 | Hs.166204 |
| 204627_s_at | 359 | 0.45 | 4.89E−05 | 142.91% | 47.23% | ITGB3 | integrin, beta 3 (platelet | Hs.87149 |
| | | | | | | | glycoprotein IIIa, antigen | |
| | | | | | | | CD61) | |
| 209674_at | 508 | 0.44 | 4.83E−10 | 74.94% | 36.71% | CRY1 | cryptochrome 1 (photolyase- | Hs.151573 |
| | | | | | | | like) | |
| 204158_s_at | 332 | 0.44 | 2.24E−09 | 60.61% | 45.60% | TCIRG1 | T-cell, immune regulator 1, | Hs.46465 |
| | | | | | | | ATPase, H+ transporting, | |
| | | | | | | | lysosomal V0 protein a isoform 3 | |
| 204731_at | 362 | 0.44 | 3.88E−08 | 89.75% | 41.63% | TGFBR3 | transforming growth factor, | Hs.342874 |
| | | | | | | | beta receptor III (betaglycan, | |
| | | | | | | | 300 kD) | |
| 222315_at | 813 | 0.44 | 1.83E−08 | 61.85% | 50.17% | | | Hs.292853 |
| 214617_at | 659 | 0.44 | 3.89E−05 | 132.11% | 54.52% | PRF1 | perforin 1 (pore forming | Hs.411106 |
| | | | | | | | protein) | |
| 211429_s_at | 558 | 0.44 | 1.47E−08 | 99.17% | 28.25% | SERPINA1 | serine (or cysteine) proteinase | Hs.297681 |
| | | | | | | | inhibitor, clade A (alpha-1 | |
| | | | | | | | antiproteinase, antitrypsin), | |
| | | | | | | | member 1 | |
| 211919_s_at | 575 | 0.44 | 1.78E−13 | 66.91% | 23.29% | CXCR4 | chemokine (C—X—C motif), | Hs.89414 |
| | | | | | | | receptor 4 (fusin) | |
| 212508_at | 600 | 0.44 | 2.82E−20 | 45.20% | 19.28% | MAP-1 | modulator of apoptosis 1 | Hs.24719 |
| 213193_x_at | 111 | 0.44 | 7.58E−07 | 118.46% | 35.66% | TRB@ | T cell receptor beta locus | Hs.303157 |
| 215275_at | 108 | 0.44 | 8.07E−11 | 85.22% | 17.38% | | | |
| 205070_at | 381 | 0.44 | 1.03E−13 | 42.45% | 35.11% | ING3 | inhibitor of growth family, | Hs.143198 |
| | | | | | | | member 3 | |
| 220890_s_at | 788 | 0.44 | 6.68E−25 | 36.96% | 16.82% | LOC51202 | hqp0256 protein | Hs.284288 |
| 210606_x_at | 543 | 0.44 | 1.80E−08 | 92.09% | 39.34% | KLRD1 | killer cell lectin-like receptor | Hs.41682 |
| | | | | | | | subfamily D, member 1 | |
| 204491_at | 352 | 0.44 | 9.84E−15 | 57.70% | 27.77% | PDE4D | phosphodiesterase 4D, cAMP- | Hs.172081 |
| | | | | | | | specific (phosphodiesterase | |
| | | | | | | | E3 dunce homolog, | |
| | | | | | | | Drosophila) | |
| 220066_at | 774 | 0.44 | 2.04E−10 | 77.28% | 35.18% | CARD15 | caspase recruitment domain | Hs.135201 |
| | | | | | | | family, member 15 | |
| 218964_at | 748 | 0.44 | 1.85E−15 | 43.77% | 31.13% | DRIL2 | dead ringer (Drosophila)-like 2 | Hs.10431 |
| | | | | | | | (bright and dead ringer) | |
| 204019_s_at | 320 | 0.44 | 2.32E−07 | 96.30% | 47.51% | DKFZP586F1318 | hypothetical protein | Hs.432325 |
| | | | | | | | DKFZP586F1318 | |
| 212400_at | 597 | 0.43 | 1.01E−10 | 83.88% | 27.30% | | | Hs.349755 |
| 219947_at | 771 | 0.43 | 2.91E−09 | 85.16% | 39.01% | CLECSF6 | C-type (calcium dependent, | Hs.115515 |
| | | | | | | | carbohydrate-recognition | |
| | | | | | | | domain) lectin, superfamily | |
| | | | | | | | member 6 | |
| 204912_at | 114 | 0.43 | 2.36E−13 | 71.20% | 22.28% | IL10RA | interleukin 10 receptor, alpha | Hs.327 |
| 204951_at | 377 | 0.43 | 6.62E−13 | 68.70% | 29.59% | ARHH | ras homolog gene family, | Hs.109918 |
| | | | | | | | member H | |
| 214049_x_at | 644 | 0.43 | 7.17E−11 | 78.15% | 33.94% | CD7 | CD7 antigen (p41) | Hs.36972 |
| 218831_s_at | 740 | 0.43 | 7.63E−09 | 101.10% | 30.44% | FCGRT | Fc fragment of IgG, receptor, | Hs.111903 |
| | | | | | | | transporter, alpha | |
| 205992_s_at | 421 | 0.43 | 4.36E−14 | 40.54% | 35.31% | IL15 | interleukin 15 | Hs.168132 |
| 60084_at | 824 | 0.43 | 4.04E−19 | 48.64% | 22.69% | CYLD | cylindromatosis (turban tumor | Hs.18827 |
| | | | | | | | syndrome) | |
| 207460_at | 452 | 0.42 | 3.62E−14 | 59.33% | 30.98% | GZMM | granzyme M (lymphocyte metase | Hs.268531 |
| | | | | | | | 1) | |
| 215666_at | 673 | 0.42 | 2.16E−03 | 118.92% | 106.86% | HLA-DRB4 | major histocompatibility | Hs.318720 |
| | | | | | | | complex, class II, DR beta 4 | |
| 217838_s_at | 699 | 0.42 | 3.55E−09 | 98.35% | 32.55% | RNB6 | RNB6 | Hs.241471 |
| 202833_s_at | 266 | 0.42 | 3.54E−08 | 110.50% | 32.29% | SERPINA1 | serine (or cysteine) proteinase | Hs.297681 |
| | | | | | | | inhibitor, clade A (alpha-1 | |
| | | | | | | | antiproteinase, antitrypsin), | |
| | | | | | | | member 1 | |
| 210915_x_at | 553 | 0.42 | 1.97E−06 | 135.65% | 35.59% | TRB@ | T cell receptor beta locus | Hs.303157 |
| 207339_s_at | 449 | 0.42 | 1.22E−06 | 126.75% | 42.23% | LTB | lymphotoxin beta (TNF | Hs.890 |
| | | | | | | | superfamily, member 3) | |
| 221724_s_at | 117 | 0.42 | 1.32E−10 | 85.44% | 33.28% | CLECSF6 | C-type (calcium dependent, | Hs.115515 |
| | | | | | | | carbohydrate-recognition | |
| | | | | | | | domain) lectin, superfamily | |
| | | | | | | | member 6 | |
| 221059_s_at | 793 | 0.42 | 6.90E−15 | 68.88% | 20.17% | CHST6 | carbohydrate (N- | Hs.157439 |
| | | | | | | | acetylglucosamine 6-O) | |
| | | | | | | | sulfotransferase 6 | |
| 209201_x_at | 488 | 0.42 | 1.63E−15 | 65.60% | 21.71% | CXCR4 | chemokine (C—X—C motif), | Hs.89414 |
| | | | | | | | receptor 4 (fusin) | |
| 212501_at | 599 | 0.42 | 8.81E−12 | 84.93% | 22.86% | CEBPB | CCAAT/enhancer binding | Hs.99029 |
| | | | | | | | protein (C/EBP), beta | |
| 201739_at | 123 | 0.42 | 1.15E−07 | 102.88% | 46.70% | SGK | serum/glucocorticoid regulated | Hs.296323 |
| | | | | | | | kinase | |
| 207072_at | 446 | 0.42 | 9.05E−10 | 77.08% | 43.43% | IL18RAP | interleukin 18 receptor | Hs.158315 |
| | | | | | | | accessory protein | |
| 200920_s_at | 176 | 0.42 | 1.24E−10 | 72.36% | 40.91% | BTG1 | B-cell translocation gene 1, | Hs.77054 |
| | | | | | | | anti-proliferative | |
| 203334_at | 291 | 0.41 | 9.88E−18 | 53.89% | 25.03% | DDX8 | DEAD/H (Asp-Glu-Ala- | Hs.171872 |
| | | | | | | | Asp/His) box polypeptide 8 | |
| | | | | | | | (RNA helicase) | |
| 204622_x_at | 358 | 0.41 | 1.60E−09 | 93.16% | 37.30% | NR4A2 | nuclear receptor subfamily 4, | Hs.82120 |
| | | | | | | | group A, member 2 | |
| 212231_at | 591 | 0.41 | 1.45E−19 | 51.15% | 21.95% | FBXO21 | F-box only protein 21 | Hs.184227 |
| 202637_s_at | 258 | 0.41 | 2.23E−11 | 72.25% | 38.03% | ICAM1 | intercellular adhesion molecule | Hs.168383 |
| | | | | | | | 1 (CD54), human rhinovirus | |
| | | | | | | | receptor | |
| 213539_at | 132 | 0.41 | 2.78E−08 | 106.66% | 39.69% | CD3D | CD3D antigen, delta | Hs.95327 |
| | | | | | | | polypeptide (TiT3 complex) | |
| 205291_at | 391 | 0.41 | 1.22E−11 | 67.18% | 38.85% | IL2RB | interleukin 2 receptor, beta | Hs.75596 |
| 202723_s_at | 261 | 0.41 | 2.90E−12 | 55.21% | 39.67% | FOXO1A | forkhead box O1A | Hs.170133 |
| | | | | | | | (rhabdomyosarcoma) | |
| 206343_s_at | 431 | 0.41 | 5.98E−10 | 55.18% | 48.19% | NRG1 | neuregulin 1 | Hs.172816 |
| 203543_s_at | 296 | 0.41 | 1.87E−10 | 92.09% | 32.00% | BTEB1 | basic transcription element | Hs.150557 |
| | | | | | | | binding protein 1 | |
| 202644_s_at | 259 | 0.41 | 5.67E−12 | 86.22% | 23.66% | TNFAIP3 | tumor necrosis factor, alpha- | Hs.211600 |
| | | | | | | | induced protein 3 | |
| 219622_at | 764 | 0.41 | 1.13E−10 | 85.10% | 35.95% | RAB20 | RAB20, member RAS | Hs.179791 |
| | | | | | | | oncogene family | |
| 219528_s_at | 762 | 0.41 | 2.09E−08 | 118.86% | 24.30% | BCL11B | B-cell CLL/lymphoma 11B | Hs.57987 |
| | | | | | | | (zinc finger protein) | |
| 217591_at | 693 | 0.41 | 2.28E−10 | 51.94% | 47.24% | | | Hs.272108 |
| 204838_s_at | 369 | 0.41 | 2.59E−10 | 38.33% | 48.54% | MLH3 | mutL homolog 3 (E. coli) | Hs.279843 |
| 213915_at | 641 | 0.41 | 4.26E−08 | 113.63% | 38.58% | NKG7 | natural killer cell group 7 | Hs.10306 |
| | | | | | | | sequence | |
| 213142_x_at | 615 | 0.40 | 3.38E−14 | 72.90% | 26.61% | LOC54103 | hypothetical protein | Hs.12969 |
| 203888_at | 312 | 0.40 | 1.09E−05 | 125.03% | 63.75% | THBD | thrombomodulin | Hs.2030 |
| 211841_s_at | 574 | 0.40 | 1.02E−12 | 83.08% | 25.18% | TNFRSF12 | tumor necrosis factor receptor | Hs.180338 |
| | | | | | | | superfamily, member 12 | |
| | | | | | | | (translocating chain- | |
| | | | | | | | association membrane protein) | |
| 204118_at | 330 | 0.40 | 9.75E−15 | 74.10% | 14.40% | CD48 | CD48 antigen (B-cell | Hs.901 |
| | | | | | | | membrane protein) | |
| 212841_s_at | 609 | 0.40 | 1.41E−07 | 48.10% | 62.68% | PPFIBP2 | PTPRF interacting protein, | Hs.12953 |
| | | | | | | | binding protein 2 (liprin beta 2) | |
| 205255_x_at | 389 | 0.40 | 4.07E−10 | 91.84% | 38.82% | TCF7 | transcription factor 7 (T-cell | Hs.169294 |
| | | | | | | | specific, HMG-box) | |
| 209871_s_at | 515 | 0.40 | 4.73E−09 | 98.50% | 42.93% | APBA2 | amyloid beta (A4) precursor | Hs.26468 |
| | | | | | | | protein-binding, family A, | |
| | | | | | | | member 2 (X11-like) | |
| 209536_s_at | 501 | 0.39 | 6.76E−15 | 55.98% | 33.99% | EHD4 | EH-domain containing 4 | Hs.4943 |
| 203708_at | 304 | 0.39 | 3.49E−11 | 95.00% | 30.17% | PDE4B | phosphodiesterase 4B, cAMP- | Hs.188 |
| | | | | | | | specific (phosphodiesterase | |
| | | | | | | | E4 dunce homolog, | |
| | | | | | | | Drosophila) | |
| 202048_s_at | 236 | 0.39 | 5.89E−16 | 63.65% | 28.85% | CBX6 | chromobox homolog 6 | Hs.107374 |
| 218205_s_at | 717 | 0.39 | 4.03E−18 | 34.91% | 30.54% | MKNK2 | MAP kinase-interacting | Hs.261828 |
| | | | | | | | serine/threonine kinase 2 | |
| 209824_s_at | 131 | 0.38 | 2.79E−13 | 73.55% | 35.30% | ARNTL | aryl hydrocarbon receptor | Hs.74515 |
| | | | | | | | nuclear translocator-like | |
| 213958_at | 102 | 0.38 | 4.17E−10 | 111.46% | 28.16% | CD6 | CD6 antigen | Hs.81226 |
| 221558_s_at | 88 | 0.38 | 8.56E−10 | 109.99% | 35.27% | LEF1 | lymphoid enhancer-binding | Hs.44865 |
| | | | | | | | factor 1 | |
| 208622_s_at | 462 | 0.38 | 4.22E−16 | 67.21% | 29.57% | VIL2 | villin 2 (ezrin) | Hs.155191 |
| 218345_at | 723 | 0.38 | 9.04E−07 | 111.02% | 62.99% | HCA112 | hepatocellular carcinoma- | Hs.12126 |
| | | | | | | | associated antigen 112 | |
| 204777_s_at | 363 | 0.38 | 5.40E−10 | 101.33% | 41.03% | MAL | mal, T-cell differentiation | Hs.80395 |
| | | | | | | | protein | |
| 213300_at | 620 | 0.37 | 9.54E−10 | 49.97% | 53.43% | KIAA0404 | KIAA0404 protein | Hs.105850 |
| 210054_at | 524 | 0.37 | 1.89E−18 | 65.35% | 23.26% | MGC4701 | hypothetical protein MGC4701 | Hs.116771 |
| 219117_s_at | 754 | 0.37 | 2.29E−10 | 97.73% | 40.82% | FKBP11 | FK506 binding protein 11 (19 kDa) | Hs.24048 |
| 204244_s_at | 339 | 0.37 | 6.56E−18 | 60.46% | 27.96% | ASK | activator of S phase kinase | Hs.152759 |
| 222142_at | 810 | 0.37 | 2.29E−22 | 50.09% | 22.95% | CYLD | cylindromatosis (turban tumor | Hs.18827 |
| | | | | | | | syndrome) | |
| 205241_at | 387 | 0.37 | 3.84E−12 | 78.99% | 39.96% | SCO2 | SCO cytochrome oxidase | Hs.278431 |
| | | | | | | | deficient homolog 2 (yeast) | |
| 202320_at | 246 | 0.37 | 5.08E−09 | 41.96% | 57.92% | GTF3C1 | general transcription factor | Hs.331 |
| | | | | | | | IIIC, polypeptide 1 (alpha | |
| | | | | | | | subunit, 220 kD) | |
| 204103_at | 328 | 0.37 | 6.82E−04 | 106.80% | 109.56% | SCYA4 | small inducible cytokine A4 | Hs.75703 |
| 211583_x_at | 565 | 0.37 | 3.06E−13 | 50.67% | 41.55% | LY117 | lymphocyte antigen 117 | Hs.88411 |
| 211962_s_at | 580 | 0.37 | 1.52E−16 | 74.42% | 25.97% | ZFP36L1 | zinc finger protein 36, C3H | Hs.85155 |
| | | | | | | | type-like 1 | |
| 204411_at | 346 | 0.37 | 1.46E−12 | 70.01% | 41.24% | KIAA0449 | KIAA0449 protein | Hs.169182 |
| 208657_s_at | 465 | 0.36 | 6.92E−19 | 66.29% | 23.55% | MSF | MLL septin-like fusion | Hs.181002 |
| 219593_at | 79 | 0.36 | 4.65E−11 | 108.68% | 31.98% | PHT2 | peptide transporter 3 | Hs.237856 |
| 222150_s_at | 811 | 0.36 | 6.54E−15 | 71.48% | 34.24% | LOC54103 | hypothetical protein | Hs.12969 |
| 201425_at | 51 | 0.36 | 1.85E−12 | 103.39% | 24.19% | ALDH2 | aldehyde dehydrogenase 2 | Hs.195432 |
| | | | | | | | family (mitochondrial) | |
| 201565_s_at | 219 | 0.36 | 1.22E−16 | 71.93% | 28.77% | ID2 | inhibitor of DNA binding 2, | Hs.180919 |
| | | | | | | | dominant negative helix-loop- | |
| | | | | | | | helix protein | |
| 209501_at | 498 | 0.36 | 1.08E−20 | 57.82% | 25.10% | CDR2 | cerebellar degeneration- | Hs.75124 |
| | | | | | | | related protein (62 kD) | |
| 221890_at | 804 | 0.36 | 6.50E−11 | 58.22% | 49.64% | ZNF335 | zinc finger protein 335 | Hs.165983 |
| 211840_s_at | 573 | 0.35 | 4.46E−15 | 59.93% | 37.12% | PDE4D | phosphodiesterase 4D, cAMP- | Hs.172081 |
| | | | | | | | specific (phosphodiesterase | |
| | | | | | | | E3 dunce homolog, | |
| | | | | | | | Drosophila) | |
| 218486_at | 726 | 0.35 | 5.27E−22 | 58.11% | 23.19% | TIEG2 | TGFB inducible early growth | Hs.12229 |
| | | | | | | | response 2 | |
| 212196_at | 590 | 0.35 | 1.52E−18 | 72.60% | 23.80% | | | Hs.71968 |
| 219359_at | 759 | 0.35 | 1.37E−12 | 82.00% | 41.21% | FLJ22635 | hypothetical protein FLJ22635 | Hs.353181 |
| 204655_at | 361 | 0.34 | 2.21E−09 | 116.09% | 47.89% | SCYA5 | small inducible cytokine A5 | Hs.241392 |
| | | | | | | | (RANTES) | |
| 206366_x_at | 432 | 0.34 | 7.78E−08 | 129.93% | 55.60% | SCYC1, | small inducible cytokine | Hs.3195 |
| | | | | | | SCYC2 | subfamily C, member 1 | |
| | | | | | | | (lymphotactin), small inducible | |
| | | | | | | | cytokine subfamily C, member 2 | |
| 214146_s_at | 646 | 0.34 | 1.46E−10 | 122.42% | 36.27% | PPBP | pro-platelet basic protein | Hs.2164 |
| | | | | | | | (includes platelet basic protein, | |
| | | | | | | | beta-thromboglobulin, | |
| | | | | | | | connective tissue-activating | |
| | | | | | | | peptide III, neutrophil- | |
| | | | | | | | activating peptide-2) | |
| 38037_at | 820 | 0.34 | 1.33E−07 | 135.13% | 56.83% | DTR | diphtheria toxin receptor | Hs.799 |
| | | | | | | | (heparin-binding epidermal | |
| | | | | | | | growth factor-like growth | |
| | | | | | | | factor) | |
| 209062_x_at | 482 | 0.34 | 9.87E−21 | 65.89% | 24.70% | NCOA3 | nuclear receptor coactivator 3 | Hs.225977 |
| 213524_s_at | 630 | 0.33 | 2.99E−10 | 105.05% | 47.78% | G0S2 | putative lymphocyte G0/G1 | Hs.432132 |
| | | | | | | | switch gene | |
| 213135_at | 614 | 0.33 | 1.80E−16 | 89.95% | 22.91% | | | Hs.82141 |
| 210479_s_at | 539 | 0.33 | 1.86E−16 | 83.74% | 29.89% | RORA | RAR-related orphan receptor A | Hs.2156 |
| 210279_at | 531 | 0.33 | 2.25E−08 | 123.27% | 56.47% | GPR18 | G protein-coupled receptor 18 | Hs.88269 |
| 1405_i_at | 155 | 0.33 | 2.64E−09 | 135.74% | 44.48% | SCYA5 | small inducible cytokine A5 | Hs.241392 |
| | | | | | | | (RANTES) | |
| 210321_at | 532 | 0.33 | 3.67E−03 | 326.10% | 90.79% | CTLA1 | similar to granzyme B | Hs.348264 |
| | | | | | | | (granzyme 2, cytotoxic T- | |
| | | | | | | | lymphocyte-associated serine | |
| | | | | | | | esterase 1) (H. sapiens) | |
| 201566_x_at | 220 | 0.33 | 2.67E−14 | 79.78% | 38.73% | ID2 | inhibitor of DNA binding 2, | Hs.180919 |
| | | | | | | | dominant negative helix-loop- | |
| | | | | | | | helix protein | |
| 204198_s_at | 336 | 0.33 | 1.17E−13 | | | RUNX3 | runt-related transcription factor 3 | Hs.170019 |
| 218696_at | 731 | 0.32 | 2.48E−23 | | | EIF2AK3 | eukaryotic translation initiation | Hs.102506 |
| | | | | | | | factor 2-alpha kinase 3 | |
| 213624_at | 152 | 0.32 | 1.74E−09 | | | | acid sphingomyelinase-like | Hs.42945 |
| | | | | | | | phosphodiesterase | |
| 218793_s_at | 736 | 0.32 | 1.17E−18 | | | SCML1 | sex comb on midleg-like 1 | Hs.109655 |
| | | | | | | | (Drosophila) | |
| 204197_s_at | 335 | 0.32 | 3.00E−17 | | | RUNX3 | runt-related transcription factor 3 | Hs.170019 |
| 209728_at | 509 | 0.32 | 2.53E−04 | 163.58% | 101.38% | HLA-DRB4 | major histocompatibility | Hs.318720 |
| | | | | | | | complex, class II, DR beta 4 | |
| 202206_at | 242 | 0.32 | 1.53E−15 | 89.61% | 32.16% | ARL7 | ADP-ribosylation factor-like 7 | Hs.111554 |
| 212195_at | 589 | 0.32 | 3.87E−17 | 90.97% | 24.26% | | | Hs.71968 |
| 206296_x_at | 428 | 0.32 | 1.58E−10 | 59.76% | 54.60% | MAP4K1 | mitogen-activated protein | Hs.95424, |
| | | | | | | | kinase kinase kinase kinase 1 | Hs.86575 |
| 201189_s_at | 193 | 0.32 | 3.76E−16 | 98.75% | 23.89% | ITPR3 | inositol 1,4,5-triphosphate | Hs.77515 |
| | | | | | | | receptor, type 3 | |
| 219099_at | 115 | 0.32 | 1.10E−20 | 66.40% | 27.62% | C12orf5 | chromosome 12 open reading | Hs.24792 |
| | | | | | | | frame 5 | |
| 210113_s_at | 527 | 0.31 | 9.95E−18 | | | NALP1 | death effector filament-forming | Hs.104305 |
| | | | | | | | Ced-4-like apoptosis protein | |
| 212187_x_at | 588 | 0.31 | 1.65E−11 | 72.81% | 50.49% | PTGDS | prostaglandin D2 synthase | Hs.8272 |
| | | | | | | | (21 kD, brain) | |
| 209604_s_at | 504 | 0.31 | 7.32E−17 | 83.69% | 32.25% | GATA3 | GATA binding protein 3 | Hs.169946 |
| 204794_at | 367 | 0.31 | 3.14E−15 | 98.27% | 32.11% | DUSP2 | dual specificity phosphatase 2 | Hs.1183 |
| 204790_at | 365 | 0.31 | 3.37E−12 | 53.77% | 49.07% | MADH7 | MAD, mothers against | Hs.100602 |
| | | | | | | | decapentaplegic homolog 7 | |
| | | | | | | | (Drosophila) | |
| 202208_s_at | 244 | 0.31 | 2.85E−11 | 97.48% | 48.91% | ARL7 | ADP-ribosylation factor-like 7 | Hs.111554 |
| 203821_at | 309 | 0.30 | 2.38E−09 | 132.98% | 52.56% | DTR | diphtheria toxin receptor | Hs.799 |
| | | | | | | | (heparin-binding epidermal | |
| | | | | | | | growth factor-like growth | |
| | | | | | | | factor) | |
| 214567_s_at | 657 | 0.30 | 7.72E−12 | 65.03% | 50.48% | SCYC1, | small inducible cytokine | Hs.174228 |
| | | | | | | SCYC2 | subfamily C, member 1 | |
| | | | | | | | (lymphotactin), small inducible | |
| | | | | | | | cytokine subfamily C, member 2 | |
| 203887_s_at | 311 | 0.30 | 1.57E−07 | 136.61% | 66.32% | THBD | thrombomodulin | Hs.2030 |
| 206655_s_at | 438 | 0.30 | 5.47E−11 | 69.52% | 53.78% | GP1BB | glycoprotein lb (platelet), beta | Hs.283743 |
| | | | | | | | polypeptide | |
| 214219_x_at | 647 | 0.30 | 2.94E−10 | 70.71% | 57.65% | MAP4K1 | mitogen-activated protein | Hs.95424, |
| | | | | | | | kinase kinase kinase kinase 1 | Hs.86575 |
| 211748_x_at | 569 | 0.29 | 6.29E−11 | | | | prostaglandin D2 synthase | Hs.8272 |
| | | | | | | | (21 kD, brain) | |
| 202988_s_at | 278 | 0.29 | 6.99E−06 | | | RGS1 | regulator of G-protein | Hs.75256 |
| | | | | | | | signalling 1 | |
| 202207_at | 243 | 0.29 | 9.60E−22 | | | ARL7 | ADP-ribosylation factor-like 7 | Hs.111554 |
| 204793_at | 366 | 0.29 | 2.70E−18 | 97.58% | 22.06% | KIAA0443 | KIAA0443 gene product | Hs.113082 |
| 214470_at | 654 | 0.29 | 1.86E−17 | 94.96% | 29.59% | KLRB1 | killer cell lectin-like receptor | Hs.169824 |
| | | | | | | | subfamily B, member 1 | |
| 210164_at | 528 | 0.29 | 1.45E−11 | 128.23% | 43.90% | GZMB | granzyme B (granzyme 2, | Hs.1051 |
| | | | | | | | cytotoxic T-lymphocyte- | |
| | | | | | | | associated serine esterase 1) | |
| 221756_at | 800 | 0.29 | 1.38E−20 | 80.93% | 27.52% | MGC17330 | hypothetical protein | Hs.26670 |
| | | | | | | | MGC17330 | |
| 206390_x_at | 433 | 0.28 | 3.02E−11 | | | PF4 | platelet factor 4 | Hs.81564 |
| 208146_s_at | 460 | 0.28 | 1.04E−17 | | | CPVL | carboxypeptidase, vitellogenic- | Hs.95594 |
| | | | | | | | like | |
| 214032_at | 642 | 0.27 | 4.56E−16 | 102.92% | 36.01% | ZAP70 | zeta-chain (TCR) associated | Hs.234569 |
| | | | | | | | protein kinase (70 kD) | |
| 216834_at | 687 | 0.27 | 9.67E−08 | 107.30% | 73.61% | RGS1 | regulator of G-protein | Hs.385701, |
| | | | | | | | signalling 1 | Hs.75256 |
| 210426_x_at | 537 | 0.26 | 4.55E−19 | 95.05% | 31.13% | RORA | RAR-related orphan receptor A | Hs.2156 |
| 220646_s_at | 783 | 0.25 | 4.98E−14 | 136.06% | 39.89% | KLRF1 | killer cell lectin-like receptor | Hs.183125 |
| | | | | | | | subfamily F, member 1 | |
| 203414_at | 294 | 0.25 | 5.84E−28 | 65.64% | 23.41% | MMD | monocyte to macrophage | Hs.79889 |
| | | | | | | | differentiation-associated | |
| 210512_s_at | 541 | 0.25 | 6.16E−11 | 77.66% | 58.76% | VEGF | vascular endothelial growth | Hs.73793 |
| | | | | | | | factor | |
| 203271_s_at | 289 | 0.24 | 1.08E−20 | 57.24% | 33.16% | UNC119 | unc-119 homolog (C. elegans) | Hs.81728 |
| 204081_at | 326 | 0.24 | 1.14E−16 | 60.84% | 40.84% | NRGN | neurogranin (protein kinase C | Hs.26944 |
| | | | | | | | substrate, RC3) | |
| 204115_at | 329 | 0.23 | 8.80E−16 | | | GNG11 | guanine nucleotide binding | Hs.83381 |
| | | | | | | | protein 11 | |
| 37145_at | 818 | 0.23 | 3.86E−12 | 161.44% | 48.15% | GNLY | granulysin | Hs.105806 |
| 205495_s_at | 398 | 0.22 | 1.07E−11 | 153.17% | 52.73% | GNLY | granulysin | Hs.105806 |
| 205237_at | 385 | 0.22 | 1.12E−17 | 131.65% | 33.86% | FCN1 | ficolin (collagen/fibrinogen | Hs.252136 |
| | | | | | | | domain containing) 1 | |
| 210031_at | 520 | 0.22 | 1.72E−21 | 106.54% | 30.59% | CD3Z | CD3Z antigen, zeta | Hs.97087 |
| | | | | | | | polypeptide (TiT3 complex) | |
| 220532_s_at | 781 | 0.21 | 3.51E−07 | 129.47% | 85.67% | LR8 | LR8 protein | Hs.190161 |
| 221211_s_at | 794 | 0.20 | 6.63E−15 | 44.22% | 46.84% | C21orf7 | chromosome 21 open reading | Hs.41267 |
| | | | | | | | frame 7 | |
| 201506_at | 213 | 0.14 | 2.13E−27 | 140.21% | 27.11% | TGFBI | transforming growth factor, | Hs.118787 |
| | | | | | | | beta-induced, 68 kD | |

Each HG-U133A qualifier represents an oligonucleotide probe set on the HG-U133A gene chip. The RNA transcript(s) of a gene that corresponds to a HG-U133A qualifier can hybridize under nucleic acid array hybridization conditions to at least one oligonucleotide probe (PM or perfect match probe) of the qualifier. Preferably, the RNA transcript(s) of the gene does not hybridize under nucleic acid array hybridization conditions to a mismatch probe (MM) of the PM probe. A mismatch probe is identical to the corresponding PM probe except for a single, homomeric substitution at or near the center of the mismatch probe. For a 25-mer PM probe, the MM probe has a homomeric base change at the 13th position.

In many cases, the RNA transcript(s) of a gene that corresponds to a HG-U133A qualifier can hybridize under nucleic acid array hybridization conditions to at least 50%, 60%, 70%, 80%, 90% or 100% of all of the PM probes of the qualifier, but not to the mismatch probes of these PM probes. In many other cases, the discrimination score (R) for each of these PM probes, as measured by the ratio of the hybridization intensity difference of the corresponding probe pair (i.e., PM−MM) over the overall hybridization intensity (i.e., PM+MM), is no less than 0.015, 0.02, 0.05, 0.1, 0.2, 0.3, 0.4, 0.5 or greater. In one example, the RNA transcript(s) of the gene, when hybridized to the HG-U133A gene chip according to the manufacturer's instructions, produces a “present” call under the default settings, i.e., the threshold Tau is 0.015 and the significance level α₁ is 0.4. See GeneChip® Expression Analysis—Data Analysis Fundamentals (Part No. 701190 Rev. 2, Affymetrix, Inc., 2002), the entire content of which is incorporated herein by reference.

The sequences of each PM probe on the HG-U133A gene chip, and the corresponding target sequences from which the PM probes are derived, can be obtained from Affymetrix's sequence databases. See, for example, www.affymetrix.com/support/technical/byproduct.affx?product=hgu133. All of these target and oligonucleotide probe sequences are incorporated herein by reference.

In addition, genes whose expression levels are significantly elevated (p<0.001) in PBMCs of AML patients relative to disease-free subjects are shown in Table 8. Genes whose expression levels are significantly lowered (p<0.001) in PBMCs of AML patients relative to disease-free subjects are shown in Table 9.

Each gene described in Tables 7, 8 and 9 and the corresponding unigene(s) are identified based on HG-U133A genechip annotations. A unigene is composed of a non-redundant set of gene-oriented clusters. Each unigene cluster is believed to include sequences that represent a unique gene. Information for each gene listed in Table 7, 8 and 9 and its corresponding unigene(s) can also be obtained from the Entrez Gene and Unigene databases at National Center for Biotechnology Information (NCBI), Bethesda, Md.

In addition to Affymetrix annotations, gene(s) that corresponds to a HG-U133A qualifier can be identified by BLAST searching the target sequence of the qualifier against a human genome sequence database. Human genome sequence databases suitable for this purpose include, but are not limited to, the NCBI human genome database. NCBI also provides BLAST programs, such as “blastn,” for searching its sequence databases. In one embodiment, the BLAST search of the NCBI human genome database is performed by using an unambiguous segment (e.g., the longest unambiguous segment) of the target sequence of the qualifier. Gene(s) that aligns to the unambiguous segment with significant sequence identity can be identified. In many cases, the identified gene(s) has at least 95%, 96%, 97%, 98%, 99%, or more sequence identity to the unambiguous segment.

As used herein, genes listed in all the Tables encompasse not only the genes that are explicitly depicted, but also genes that are not listed in the table but nonetheless corresponds to a qualifier in the table. All of these genes can be used as biological markers for the diagnosis or monitoring the development, progression or treatment of AML.

TABLE 8
Top 50 transcripts at significantly elevated levels (p < 0.001)
in PBMCs of AML patients relative to disease-free subjects

| | | | | | AML | Normal | | |
|---|---|---|---|---|---|---|---|---|
| | | | | | Average | Average | Fold Diff | p-value |
| Affymetrix ID | SEQ ID NO: | Name | Cyto Band | Unigene ID | (ppm) | (ppm) | AML/Norm | (unequal) |
| 203948_s_at | 316 | myeloperoxidase | 17q23.1 | Hs.1817 | 83.00 | 1.78 | 46.69 | 4.63E−06 |
| 203949_at | 317 | myeloperoxidase | 17q23.1 | Hs.1817 | 74.97 | 2.13 | 35.14 | 1.19E−06 |
| 206310_at | 429 | serine protease inhibitor, Kazal type, | 4q11 | Hs.98243 | 43.47 | 1.91 | 22.75 | 3.86E−06 |
| | | 2 (acrosin-trypsin inhibitor) | | | | | | |
| 209905_at | 518 | homeo box A9 | 7p15-p14 | Hs.127428 | 21.08 | 1.00 | 21.08 | 5.44E−05 |
| 214575_s_at | 658 | azurocidin 1 (cationic antimicrobial | 19p13.3 | Hs.72885 | 36.92 | 1.84 | 20.02 | 3.88E−04 |
| | | protein 37) | | | | | | |
| 206871_at | 444 | elastase 2, neutrophil | 19p13.3 | Hs.99863 | 35.58 | 1.93 | 18.41 | 1.23E−04 |
| 214651_s_at | 660 | homeo box A9 | 7p15-p14 | Hs.127428 | 29.61 | 1.82 | 16.25 | 5.98E−05 |
| 210084_x_at | 525 | tryptase beta 1, tryptase, alpha | 16p13.3 | Hs.347933 | 14.50 | 1.02 | 14.18 | 1.20E−04 |
| 205683_x_at | 411 | tryptase beta 1, tryptase beta 2, | 16p13.3 | Hs.347933 | 20.42 | 1.47 | 13.92 | 4.32E−04 |
| | | tryptase, alpha | | | | | | |
| 204798_at | 368 | v-myb myeloblastosis viral oncogene | 6q22-q23 | Hs.1334 | 35.69 | 2.76 | 12.95 | 7.41E−10 |
| | | homolog (avian) | | | | | | |
| 217023_x_at | 688 | tryptase beta 1, tryptase beta 2 | 16p13.3 | Hs.294158, | 13.08 | 1.09 | 12.02 | 1.41E−04 |
| | | | | Hs.347933 | | | | |
| 216474_x_at | 681 | tryptase beta 1, tryptase beta 2 | 16p13.3 | Hs.347933 | 18.92 | 1.71 | 11.06 | 8.25E−05 |
| 202016_at | 235 | mesoderm specific transcript | 7q32 | Hs.79284 | 34.28 | 3.11 | 11.02 | 3.63E−04 |
| | | homolog (mouse) | | | | | | |
| 207134_x_at | 447 | tryptase beta 1, tryptase beta 2, | 16p13.3 | Hs.294158 | 17.75 | 1.62 | 10.94 | 6.98E−04 |
| | | tryptase, alpha | | | | | | |
| 215382_x_at | 670 | tryptase beta 1, tryptase, alpha | 16p13.3 | Hs.347933 | 15.19 | 1.40 | 10.85 | 5.25E−05 |
| 205950_s_at | 420 | carbonic anhydrase I | 8q13-q22.1 | Hs.23118 | 101.03 | 9.31 | 10.85 | 5.23E−04 |
| 205051_s_at | 380 | v-kit Hardy-Zuckerman 4 feline | 4q11-q12 | Hs.81665 | 16.39 | 1.60 | 10.24 | 2.37E−05 |
| | | sarcoma viral oncogene homolog | | | | | | |
| 211709_s_at | 566 | stem cell growth factor; lymphocyte | 19q13.3 | Hs.105927 | 32.19 | 3.20 | 10.06 | 1.23E−06 |
| | | secreted C-type lectin | | | | | | |
| 205131_x_at | 383 | stem cell growth factor; lymphocyte | 19q13.3 | Hs.105927 | 12.31 | 1.29 | 9.55 | 1.02E−04 |
| | | secreted C-type lectin | | | | | | |
| 219054_at | 753 | hypothetical protein FLJ14054 | 5p13.2 | Hs.13528 | 14.61 | 1.76 | 8.32 | 2.05E−06 |
| 204304_s_at | 340 | prominin-like 1 (mouse) | 4p15.33 | Hs.112360 | 12.47 | 1.62 | 7.69 | 4.74E−07 |
| 206674_at | 440 | fms-related tyrosine kinase 3 | 13q12 | Hs.385 | 15.97 | 2.16 | 7.41 | 2.90E−07 |
| 207741_x_at | 456 | tryptase, alpha | 16p13.3 | Hs.334455 | 14.33 | 1.96 | 7.33 | 5.05E−05 |
| 202589_at | 257 | thymidylate synthetase | 18p11.32 | Hs.82962 | 32.89 | 4.64 | 7.08 | 1.63E−05 |
| 210783_x_at | 549 | stem cell growth factor; lymphocyte | 19q13.3 | Hs.105927 | 7.31 | 1.04 | 6.99 | 5.96E−05 |
| | | secreted C-type lectin | | | | | | |
| 211922_s_at | 576 | catalase | 11p13 | Hs.76359 | 38.47 | 5.73 | 6.71 | 1.13E−07 |
| 201427_s_at | 208 | selenoprotein P, plasma, 1 | 5q31 | Hs.3314 | 6.64 | 1.00 | 6.64 | 7.13E−04 |
| 206111_at | 424 | ribonuclease, RNase A family, 2 | 14q24-q31 | Hs.728 | 63.06 | 9.56 | 6.60 | 2.95E−05 |
| | | (liver, eosinophil-derived neurotoxin) | | | | | | |
| 202503_s_at | 255 | KIAA0101 gene product | 15q22.1 | Hs.81892 | 25.86 | 4.04 | 6.39 | 2.92E−06 |
| 220377_at | 778 | HSPC053 protein | 14q32.33 | Hs.128155 | 6.28 | 1.02 | 6.14 | 1.93E−04 |
| 201310_s_at | 200 | P311 protein | 5q21.3 | Hs.142827 | 29.44 | 4.98 | 5.92 | 2.13E−09 |
| 219672_at | 767 | erythroid associated factor | 16p11.1 | Hs.274309 | 28.78 | 4.91 | 5.86 | 9.81E−04 |
| 205624_at | 409 | carboxypeptidase A3 (mast cell) | 3q21-q25 | Hs.646 | 20.11 | 3.56 | 5.66 | 9.30E−05 |
| 205609_at | 407 | angiopoietin 1 | 8q22.3-q23 | Hs.2463 | 6.83 | 1.22 | 5.59 | 1.49E−06 |
| 206834_at | 442 | hemoglobin, delta | 11p15.5 | Hs.36977 | 183.31 | 33.40 | 5.49 | 5.46E−05 |
| 201162_at | 192 | insulin-like growth factor binding | 4q12 | Hs.119206 | 17.72 | 3.38 | 5.25 | 3.09E−07 |
| | | protein 7 | | | | | | |
| 201432_at | 209 | catalase | 11p13 | Hs.76359 | 121.17 | 23.38 | 5.18 | 1.43E−09 |
| 204430_s_at | 8 | solute carrier family 2 (facilitated | 1p36.2 | Hs.33084 | 5.86 | 1.13 | 5.17 | 6.73E−04 |
| | | glucose/fructose transporter), | | | | | | |
| | | member 5 | | | | | | |
| 220416_at | 780 | KIAA1939 protein | 15q15.2 | Hs.182738 | 9.64 | 1.87 | 5.16 | 1.24E−06 |
| 211743_s_at | 568 | proteoglycan 2, bone marrow | 11q12 | Hs.99962 | 7.58 | 1.53 | 4.95 | 7.28E−04 |
| | | (natural killer cell activator, | | | | | | |
| | | eosinophil granule major basic | | | | | | |
| | | protein) | | | | | | |
| 201416_at | 206 | Meis1, myeloid ecotropic viral | 17p11.2, | Hs.83484 | 30.64 | 6.20 | 4.94 | 1.01E−04 |
| | | integration site 1 homolog 3 | 6p22.3 | | | | | |
| | | (mouse), SRY (sex determining | | | | | | |
| | | region Y)-box 4 | | | | | | |
| 213150_at | 617 | homeo box A10 | 7p15-p14 | Hs.110637 | 8.39 | 1.71 | 4.90 | 3.44E−04 |
| 209543_s_at | 502 | CD34 antigen, FLJ00005 protein | 15, 1q32 | Hs.367690 | 11.39 | 2.33 | 4.88 | 6.90E−07 |
| 213258_at | 65 | unknown | | Hs.288582 | 5.25 | 1.09 | 4.82 | 2.40E−07 |
| 210664_s_at | 546 | tissue factor pathway inhibitor | 2q31-q32.1 | Hs.170279 | 5.89 | 1.24 | 4.73 | 8.77E−06 |
| | | (lipoprotein-associated coagulation | | | | | | |
| | | inhibitor) | | | | | | |
| 206067_s_at | 423 | Wilms tumor 1 | 11p13 | Hs.1145 | 4.72 | 1.00 | 4.72 | 2.81E−04 |
| 209757_s_at | 70 | v-myc myelocytomatosis viral related | 2p24.1 | Hs.25960 | 4.69 | 1.00 | 4.69 | 8.72E−06 |
| | | oncogene, neuroblastoma derived | | | | | | |
| | | (avian) | | | | | | |
| 213515_x_at | 629 | glycyl-tRNA synthetase, hemoglobin, | 11p15.5, 7p15 | Hs.283108 | 345.06 | 73.71 | 4.68 | 2.22E−05 |
| | | gamma A, hemoglobin, gamma G | | | | | | |
| 219837_s_at | 769 | cytokine-like protein C17 | 4p16-p15 | Hs.13872 | 5.72 | 1.24 | 4.60 | 2.68E−04 |
| 218899_s_at | 746 | brain and acute leukemia, | 8q22.3 | Hs.169395 | 6.19 | 1.36 | 4.57 | 9.36E−04 |
| | | cytoplasmic | | | | | | |

TABLE 9
Top 50 transcripts at significantly lower levels (p < 0.001)
in PBMCs of AML patients relative to disease-free subjects

| | | | | | AML | Normal | | |
|---|---|---|---|---|---|---|---|---|
| | | | | | Average | Average | Fold Diff | p-value |
| Affymetrix | SEQ ID NO: | Name | Cyto Band | Unigene ID | (ppm) | (ppm) | Norm/AML | (unequal) |
| 201506_at | 213 | transforming growth factor, beta- | 5q31 | Hs.118787 | 6.56 | 47.31 | 7.22 | 2.13E−27 |
| | | induced, 68 kD | | | | | | |
| 221211_s_at | 794 | chromosome 21 open reading | 21q22.3 | Hs.41267 | 2.44 | 11.93 | 4.88 | 6.63E−15 |
| | | frame 7 | | | | | | |
| 220532_s_at | 781 | LR8 protein | 7q35 | Hs.190161 | 3.00 | 14.02 | 4.67 | 3.51E−07 |
| 210031_at | 520 | CD3Z antigen, zeta polypeptide | 1q22-q23 | Hs.97087 | 11.72 | 53.98 | 4.60 | 1.72E−21 |
| | | (TiT3 complex) | | | | | | |
| 205237_at | 385 | ficolin (collagen/fibrinogen domain | 9q34 | Hs.252136 | 29.56 | 132.64 | 4.49 | 1.12E−17 |
| | | containing) 1 | | | | | | |
| 205495_s_at | 398 | granulysin | 2p12-q11 | Hs.105806 | 12.86 | 57.69 | 4.49 | 1.07E−11 |
| 37145_at | 818 | granulysin | 2p12-q11 | Hs.105806 | 14.22 | 62.47 | 4.39 | 3.86E−12 |
| 204115_at | 329 | guanine nucleotide binding protein | 7q31-q32 | Hs.83381 | 2.75 | 11.80 | 4.29 | 8.80E−16 |
| | | 11 | | | | | | |
| 204081_at | 326 | neurogranin (protein kinase C | 11q24 | Hs.26944 | 7.83 | 32.69 | 4.17 | 1.14E−16 |
| | | substrate, RC3) | | | | | | |
| 203271_s_at | 289 | unc-119 homolog (C. elegans) | 17q11.2 | Hs.81728 | 1.58 | 6.60 | 4.17 | 1.08E−20 |
| 210512_s_at | 541 | vascular endothelial growth factor | 6p12 | Hs.73793 | 3.00 | 12.18 | 4.06 | 6.16E−11 |
| 203414_at | 294 | monocyte to macrophage | 17q | Hs.79889 | 7.78 | 31.47 | 4.05 | 5.84E−28 |
| | | differentiation-associated | | | | | | |
| 220646_s_at | 783 | killer cell lectin-like receptor | 12p12.3-13.2 | Hs.183125 | 4.36 | 17.51 | 4.02 | 4.98E−14 |
| | | subfamily F, member 1 | | | | | | |
| 210426_x_at | 537 | RAR-related orphan receptor A | 15q21-q22 | Hs.2156 | 4.17 | 15.78 | 3.79 | 4.55E−19 |
| 216834_at | 687 | regulator of G-protein signalling 1 | 1q31 | Hs.75256 | 10.50 | 38.56 | 3.67 | 9.67E−08 |
| 214032_at | 642 | zeta-chain (TCR) associated protein | 2q12 | Hs.234569 | 4.78 | 17.49 | 3.66 | 4.56E−16 |
| | | kinase (70 kD) | | | | | | |
| 206390_x_at | 433 | platelet factor 4 | 4q12-q21 | Hs.81564 | 16.11 | 58.53 | 3.63 | 3.02E−11 |
| 208146_s_at | 460 | carboxypeptidase, vitellogenic-like | 7p15-p14 | Hs.95594 | 10.75 | 38.51 | 3.58 | 1.04E−17 |
| 221756_at | 800 | hypothetical protein MGC17330 | 22q11.2-q22 | Hs.26670 | 13.81 | 47.98 | 3.48 | 1.38E−20 |
| 210164_at | 528 | granzyme B (granzyme 2, cytotoxic | 14q11.2 | Hs.1051 | 8.28 | 28.60 | 3.46 | 1.45E−11 |
| | | T-lymphocyte-associated serine | | | | | | |
| | | esterase 1) | | | | | | |
| 211748_x_at | 569 | prostaglandin D2 synthase (21 kD, | 9q34.2-q34.3 | Hs.8272 | 5.36 | 18.47 | 3.44 | 6.29E−11 |
| | | brain) | | | | | | |
| 202988_s_at | 278 | regulator of G-protein signalling 1 | 1q31 | Hs.75256 | 2.58 | 8.89 | 3.44 | 6.99E−06 |
| 202207_at | 243 | ADP-ribosylation factor-like 7 | 2q37.2 | Hs.111554 | 20.22 | 69.47 | 3.44 | 9.60E−22 |
| 214470_at | 654 | killer cell lectin-like receptor | 12p13 | Hs.169824 | 18.14 | 61.67 | 3.40 | 1.86E−17 |
| | | subfamily B, member 1 | | | | | | |
| 204793_at | 366 | KIAA0443 gene product | Xq22.1 | Hs.113082 | 4.81 | 16.31 | 3.39 | 2.70E−18 |
| 214219_x_at | 647 | mitogen-activated protein kinase | 19q13.1-q13.4 | Hs.86575 | 2.00 | 6.78 | 3.39 | 2.94E−10 |
| | | kinase kinase kinase 1 | | | | | | |
| 206655_s_at | 438 | glycoprotein lb (platelet), beta | 22q11.21 | Hs.283743 | 2.36 | 7.82 | 3.31 | 5.47E−11 |
| | | polypeptide | | | | | | |
| 203887_s_at | 311 | thrombomodulin | 20p12-cen | Hs.2030 | 4.28 | 14.13 | 3.30 | 1.57E−07 |
| 214567_s_at | 657 | small inducible cytokine subfamily | 1q23, 1q23-q25 | Hs.174228 | 1.39 | 4.58 | 3.30 | 7.72E−12 |
| | | C, member 1 (lymphotactin), small | | | | | | |
| | | inducible cytokine subfamily C, | | | | | | |
| | | member 2 | | | | | | |
| 203821_at | 309 | diphtheria toxin receptor (heparin- | 5q23 | Hs.799 | 11.81 | 38.84 | 3.29 | 2.38E−09 |
| | | binding epidermal growth factor-like | | | | | | |
| | | growth factor) | | | | | | |
| 202208_s_at | 244 | ADP-ribosylation factor-like 7 | 2q37.2 | Hs.111554 | 8.67 | 28.07 | 3.24 | 2.85E−11 |
| 204790_at | 365 | MAD, mothers against | 18q21.1 | Hs.100602 | 2.81 | 9.07 | 3.23 | 3.37E−12 |
| | | decapentaplegic homolog 7 | | | | | | |
| | | (Drosophila) | | | | | | |
| 210113_s_at | 527 | death effector filament-forming Ced- | 17p13 | Hs.104305 | 3.61 | 11.64 | 3.22 | 9.95E−18 |
| | | 4-like apoptosis protein | | | | | | |
| 204794_at | 367 | dual specificity phosphatase 2 | 2q11 | Hs.1183 | 7.64 | 24.51 | 3.21 | 3.14E−15 |
| 209604_s_at | 504 | GATA binding protein 3 | 10p15 | Hs.169946 | 7.36 | 23.60 | 3.21 | 7.32E−17 |
| 212187_x_at | 588 | prostaglandin D2 synthase (21 kD, | 9q34.2-q34.3 | Hs.8272 | 4.03 | 12.91 | 3.21 | 1.65E−11 |
| | | brain) | | | | | | |
| 219099_at | 115 | chromosome 12 open reading | 12p13.3 | Hs.24792 | 3.78 | 11.96 | 3.16 | 1.10E−20 |
| | | frame 5 | | | | | | |
| 201189_s_at | 193 | inositol 1,4,5-triphosphate receptor, | 6p21 | Hs.77515 | 2.94 | 9.31 | 3.16 | 3.76E−16 |
| | | type 3 | | | | | | |
| 206296_x_at | 428 | mitogen-activated protein kinase | 19q13.1-q13.4 | Hs.86575 | 2.86 | 8.96 | 3.13 | 1.58E−10 |
| | | kinase kinase kinase 1 | | | | | | |
| 212195_at | 589 | Unknown | N/a | Hs.71968 | 8.11 | 25.33 | 3.12 | 3.87E−17 |
| 218696_at | 731 | eukaryotic translation initiation | 2p12 | Hs.102506 | 6.86 | 21.42 | 3.12 | 2.48E−23 |
| | | factor 2-alpha kinase 3 | | | | | | |
| 213624_at | 152 | acid sphingomyelinase-like | 6 | Hs.42945 | 2.19 | 6.82 | 3.11 | 1.74E−09 |
| | | phosphodiesterase | | | | | | |
| 202206_at | 242 | ADP-ribosylation factor-like 7 | 2q37.2 | Hs.111554 | 14.14 | 43.80 | 3.10 | 1.53E−15 |
| 209728_at | 509 | major histocompatibility complex, | 6p21.3 | Hs.318720 | 11.25 | 34.69 | 3.08 | 2.53E−04 |
| | | class II, DR beta 4 | | | | | | |
| 218793_s_at | 736 | sex comb on midleg-like 1 | Xp22.2-p22.1 | Hs.109655 | 2.03 | 6.24 | 3.08 | 1.17E−18 |
| | | (Drosophila) | | | | | | |
| 204197_s_at | 335 | runt-related transcription factor 3 | 1p36 | Hs.170019 | 19.69 | 60.64 | 3.08 | 3.00E−17 |
| 201566_x_at | 220 | inhibitor of DNA binding 2, | 2p25 | Hs.180919 | 5.64 | 17.31 | 3.07 | 2.67E−14 |
| | | dominant negative helix-loop-helix | | | | | | |
| | | protein | | | | | | |
| 204198_s_at | 336 | runt-related transcription factor 3 | 1p36 | Hs.170019 | 12.08 | 37.00 | 3.06 | 1.17E−13 |
| 1405_i_at | 155 | small inducible cytokine A5 | 17q11.2-q12 | Hs.241392 | 11.69 | 35.67 | 3.05 | 2.64E−09 |
| | | (RANTES) | | | | | | |
| 210279_at | 531 | G protein-coupled receptor 18 | 13q32 | Hs.88269 | 4.28 | 13.02 | 3.04 | 2.25E−08 |

### Prognosis, Diagnosis and Selection of Treatment of AML or Other Leukemias

The prognostic genes of the present invention can be used for the prediction of clinical outcome of a leukemia patient of interest. The prediction typically involves comparison of the peripheral blood expression profile of one or more prognostic genes in the leukemia patient of interest to at least one reference expression profile. Each prognostic gene employed in the present invention is differentially expressed in peripheral blood samples of leukemia patients who have different clinical outcomes.

In one embodiment, the prognostic genes employed for the outcome prediction are selected such that the peripheral blood expression profile of each prognostic gene is correlated with a class distinction under a class-based correlation analysis (such as the nearest-neighbor analysis), where the class distinction represents an idealized expression pattern of the selected genes in peripheral blood samples of leukemia patients who have different clinical outcomes. In many cases, the selected prognostic genes are correlated with the class distinction at above the 50%, 25%, 10%, 5%, or 1% significance level under a random permutation test.

The prognostic genes can also be selected such that the average expression profile of each prognostic gene in peripheral blood samples of one class of leukemia patients is statistically different from that in another class of leukemia patients. For instance, the p-value under a Student's t-test for the observed difference can be no more than 0.05, 0.01, 0.005, 0.001, or less. In addition, the prognostic genes can be selected such that the average peripheral blood expression level of each prognostic gene in one class of patients is at least 2-, 3-, 4-, 5-, 10-, or 20-fold different from that in another class of patients.

The expression profile of a patient of interest can be compared to one or more reference expression profiles. The reference expression profiles can be determined concurrently with the expression profile of the patient of interest. The reference expression profiles can also be predetermined or prerecorded in electronic or other types of storage media.

The reference expression profiles can include average expression profiles, or individual profiles representing peripheral blood gene expression patterns in particular patients. In one embodiment, the reference expression profiles include an average expression profile of the prognostic gene(s) in peripheral blood samples of reference leukemia patients who have known or determinable clinical outcome. Any averaging method may be used, such as arithmetic means, harmonic means, average of absolute values, average of log-transformed values, or weighted average. In one example, the reference leukemia patients have the same clinical outcome. In another example, the reference leukemia patients can be divided into at least two classes, each class of patients having a different respective clinical outcome. The average peripheral blood expression profile in each class of patients constitutes a separate reference expression profile, and the expression profile of the patient of interest is compared to each of these reference expression profiles.

In another embodiment, the reference expression profiles includes a plurality of expression profiles, each of which represents the peripheral blood expression pattern of the prognostic gene(s) in a particular leukemia patient whose clinical outcome is known or determinable. Other types of reference expression profiles can also be used in the present invention. In yet another embodiment, the present invention uses a numerical threshold as a control level.

The expression profile of the patient of interest and the reference expression profile(s) can be constructed in any form. In one embodiment, the expression profiles comprise the expression level of each prognostic gene used in outcome prediction. The expression levels can be absolute, normalized, or relative levels. Suitable normalization procedures include, but are not limited to, those used in nucleic acid array gene expression analyses or those described in Hill, et al., GENOME BIOL, 2:research0055.1-0055.13 (2001). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodologies.

In another embodiment, each expression profile being compared comprises one or more ratios between the expression levels of different prognostic genes. An expression profile can also include other measures that are capable of representing gene expression patterns.

The peripheral blood samples used in the present invention can be either whole blood samples, or samples comprising enriched PBMCs. In one example, the peripheral blood samples used for preparing the reference expression profile(s) comprise enriched or purified PBMCs, and the peripheral blood sample used for preparing the expression profile of the patient of interest is a whole blood sample. In another example, all of the peripheral blood samples employed in outcome prediction comprise enriched or purified PBMCs. In many cases, the peripheral blood samples are prepared from the patient of interest and reference patients using the same or comparable procedures.

Other types of blood samples can also be employed in the present invention, and the gene expression profiles in these blood samples are statistically significantly correlated with patient outcome.

The peripheral blood samples used in the present invention can be isolated from respective patients at any disease or treatment stage, and the correlation between the gene expression patterns in these peripheral blood samples and clinical outcome is statistically significant. In many embodiments, clinical outcome is measured by patients' response to a therapeutic treatment, and all of the blood samples used in outcome prediction are isolated prior to the therapeutic treatment. The expression profiles derived from these blood samples are therefore baseline expression profiles for the therapeutic treatment.

Construction of the expression profiles typically involves detection of the expression level of each prognostic gene used in the outcome prediction. Numerous methods are available for this purpose. For instance, the expression level of a gene can be determined by measuring the level of the RNA transcript(s) of the gene. Suitable methods include, but are not limited to, quantitative RT-PCT, Northern Blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a prognostic gene is determined by measuring the RNA transcript level of the gene in a peripheral blood sample. RNA can be isolated from the peripheral blood sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR(RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo (dT) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a prognostic gene of interest. Quantitative RT-PCR involves reverse transcription (RT) of RNA to cDNA followed by relative quantitative PCR(RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a prognostic gene of interest. The nucleic acid arrays can be commercial oligonucleotide or cDNA arrays. They can also be custom arrays comprising concentrated probes for the prognostic genes of the present invention. In many examples, at least 15%, 20%, 25%, 30%, 35%, 40%, 45%, 50%, or more of the total probes on a custom array of the present invention are probes for leukemia prognostic genes. These probes can hybridize under stringent or nucleic acid array hybridization conditions to the RNA transcripts, or the complements thereof, of the corresponding prognostic genes.

As used herein, “stringent conditions” are at least as stringent as, for example, conditions G-L shown in Table 10. “Highly stringent conditions” are at least as stringent as conditions A-F shown in Table 10. Hybridization is carried out under the hybridization conditions (Hybridization Temperature and Buffer) for about four hours, followed by two 20-minute washes under the corresponding wash conditions (Wash Temp. and Buffer).

TABLE 10
Stringency Conditions

| | Poly- | Hybrid | Hybridization | |
|---|---|---|---|---|
| Stringency | nucleotide | Length | Temperature and | Wash Temp. |
| Condition | Hybrid | (bp)¹ | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ (° C.) = 81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M).

In one example, a nucleic acid array of the present invention includes at least 2, 5, 10, or more different probes. Each of these probes is capable of hybridizing under stringent or nucleic acid array hybridization conditions to a different respective prognostic gene of the present invention. Multiple probes for the same prognostic gene can be used on the same nucleic acid array. The probe density on the array can be in any range.

The probes for a prognostic gene of the present invention can be a nucleic acid probe, such as, DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the prognostic genes can be stably attached to discrete regions on a nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the prognostic genes of the present invention can be prepared by using any method known in the art. For prognostic genes whose genomic locations have not been determined or whose identities are solely based on EST or mRNA data, the probes/primers for these genes can be derived from the target sequences of the corresponding qualifiers, or the corresponding EST or mRNA sequences.

In one embodiment, the probes/primers for a prognostic gene significantly diverge from the sequences of other prognostic genes. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another embodiment, the probes for prognostic genes can be polypeptide in nature, such as, antibody probes. The expression levels of the prognostic genes of the present invention are thus determined by measuring the levels of polypeptides encoded by the prognostic genes. Methods suitable for this purpose include, but are not limited to, immunoassays such as ELISA, RIA, FACS, dot blot, Western Blot, immunohistochemistry, and antibody-based radioimaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding prognostic gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the prognostic genes. Methods for making protein arrays or biochips are well known in the art. In many embodiments, a substantial portion of probes on a protein array of the present invention are antibodies specific for the prognostic gene products. For instance, at least 10%, 20%, 30%, 40%, 50%, or more probes on the protein array can be antibodies specific for the prognostic gene products.

In yet another aspect, the expression levels of the prognostic genes are determined by measuring the biological functions or activities of these genes. Where a biological function or activity of a gene is known, suitable in vitro or in vivo assays can be developed to evaluate the function or activity. These assays can be subsequently used to assess the level of expression of the prognostic gene.

After the expression level of each prognostic gene is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a prognostic gene, a ratio between the expression levels of two prognostic genes, or another measure capable of representing gene expression patterns. The expression level of a gene can have an absolute or a normalized or relative value. The difference between two corresponding components can be assessed by fold changes, absolute differences, or other suitable means.

Comparison of the expression profile of a patient of interest to the reference expression profile(s) can also be conducted using pattern recognition or comparison programs, such as the k-nearest-neighbors algorithm as described in Armstrong, et al., NATURE GENETICS, 30:41-47 (2002), or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple prognostic genes can be used in the comparison of expression profiles. For instance, 2, 4, 6, 8, 10, 12, 14, or more prognostic genes can be used. In addition, the prognostic gene(s) used in the comparison can be selected to have relatively small p-values (e.g., two-sided p-values). In many examples, the p-values indicate the statistical significance of the difference between gene expression levels in different classes of patients. In many other examples, the p-values suggest the statistical significance of the correlation between gene expression patterns and clinical outcome. In one embodiment, the prognostic genes used in the comparison have p-values of no greater than 0.05, 0.01, 0.001, 0.0005, 0.0001, or less. Prognostic genes with p-values of greater than 0.05 can also be used. These genes may be identified, for instance, by using a relatively small number of blood samples.

Similarity or difference between the expression profile of a patient of interest and a reference expression profile is indicative of the class membership of the patient of interest. Similarity or difference can be determined by any suitable means. The comparison can be qualitative, quantitative, or both.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The prognostic gene(s) and the similarity criteria can be selected such that the accuracy of outcome prediction (the ratio of correct calls over the total of correct and incorrect calls) is relatively high. For instance, the accuracy of prediction can be at least 50%, 60%, 70%, 80%, 90%, or more.

The effectiveness of outcome prediction can also be assessed by sensitivity and specificity. The prognostic genes and the comparison criteria can be selected such that both the sensitivity and specificity of outcome prediction are relatively high. For instance, the sensitivity and specificity can be at least 50%, 60%, 70%, 80%, 90%, 95%, or more. As used herein, “sensitivity” refers to the ratio of correct positive calls over the total of true positive calls plus false negative calls, and “specificity” refers to the ratio of correct negative calls over the total of true negative calls plus false positive calls.

Moreover, peripheral blood expression profile-based outcome prediction can be combined with other clinical evidence or prognostic methods to improve the effectiveness or accuracy of outcome prediction.

In many embodiments, the expression profile of a patient of interest is compared to at least two reference expression profiles. Each reference expression profile can include an average expression profile, or a set of individual expression profiles each of which represents the peripheral blood gene expression pattern in a particular AML patient or disease-free human. Suitable methods for comparing one expression profile to two or more reference expression profiles include, but are not limited to, the weighted voting algorithm or the k-nearest-neighbors algorithm. Softwares capable of performing these algorithms include, but are not limited to, GeneCluster 2 software. GeneCluster 2 software is available from MIT Center for Genome Research at Whitehead Institute (e.g., wwwgenome.wi.mit.edu/cancer/software/genecluster2/gc2.html).

Both the weighted voting and k-nearest-neighbors algorithms employ gene classifiers that can effectively assign a patient of interest to an outcome class. By “effectively,” it means that the class assignment is statistically significant. In one example, the effectiveness of class assignment is evaluated by leave-one-out cross validation or k-fold cross validation. The prediction accuracy under these cross validation methods can be, for instance, at least 50%, 60%, 70%, 80%, 90%, 95%, or more. The prediction sensitivity or specificity under these cross validation methods can also be at least 50%, 60%, 70%, 80%, 90%, 95%, or more. Prognostic genes or class predictors with low assignment sensitivity/specificity or low cross validation accuracy, such as less than 50%, can also be used in the present invention.

Under one version of the weighted voting algorithm, each gene in a class predictor casts a weighted vote for one of the two classes (class 0 and class 1). The vote of gene “g” can be defined as v_{g}=a_{g} (x_{g}−b_{g}), wherein a_{g} equals to P(g,c) and reflects the correlation between the expression level of gene “g” and the class distinction between the two classes, b_{g} is calculated as b_{g}=[x0(g)+x1(g)]/2 and represents the average of the mean logs of the expression levels of gene “g” in class 0 and class 1, and x_{g} is the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1). Thus, the prediction strength varies between −1 and 1 and can indicate the support for one class (e.g., positive PS) or the other (e.g., negative PS). A prediction strength near “0” suggests narrow margin of victory, and a prediction strength close to “1” or “−1” indicates wide margin of victory. See Slonim, et al., PROCS. OF THE FOURTH ANNUAL INTERNATIONAL CONFERENCE ON COMPUTATIONAL MOLECULAR BIOLOGY, Tokyo, Japan, April 8-11, p 263-272 (2000); and Golub, et al., SCIENCE, 286: 531-537 (1999).

Suitable prediction strength (PS) thresholds can be assessed by plotting the cumulative cross-validation error rate against the prediction strength. In one embodiment, a positive predication is made if the absolute value of PS for the sample of interest is no less than 0.3. Other PS thresholds, such as no less than 0.1, 0.2, 0.4 or 0.5, can also be selected for class prediction. In many embodiments, a threshold is selected such that the accuracy of prediction is optimized and the incidence of both false positive and false negative results is minimized.

Any class predictor constructed according to the present invention can be used for the class assignment of a leukemia patient of interest. In many examples, a class predictor employed in the present invention includes n prognostic genes identified by the neighborhood analysis, where n is an integer greater than 1. A half of these prognostic genes has the largest P(g,c) scores, and the other half has the largest −P(g,c) scores. The number n therefore is the only free parameter in defining the class predictor.

The expression profile of a patient of interest can also be compared to two or more reference expression profiles by other means. For instance, the reference expression profiles can include an average peripheral blood expression profile for each class of patients. The fact that the expression profile of a patient of interest is more similar to one reference profile than to another suggests that the patient of interest is more likely to have the clinical outcome associated with the former reference profile than that associated with the latter reference profile.

In one particular embodiment, the present invention features prediction of clinical outcome of an AML patient of interest. AML patients can be divided into at least two classes based on their responses to a specified treatment regime. One class of patients (responders) has complete remission in response to the treatment, and the other class of patients (non-responders) has non-remission or partial remission in response to the treatment. AML prognostic genes that are correlated with a class distinction between these two classes of patients can be identified and then used to assign the patient of interest to one of these two outcome classes. Examples of AML prognostic genes suitable for this purpose are depicted in Tables 1 and 2.

In one example, the treatment regime includes administration of at least one chemotherapy agent (e.g., daunorubicin or cytarabine) and an anti-CD33 antibody conjugated with a cytotoxic agent (e.g., gemtuzumab ozogamicin), and the expression profile of an AML patient of interest is compared to two or more reference expression profiles by using a weighted voting or k-nearest-neighbors algorithm. All of these expression profiles are baseline profiles representing peripheral blood gene expression patterns prior to the treatment regime. A classifier including at least one gene selected from Table 1 and at least one gene selected from Table 2 can be employed for the outcome prediction. For instance, a classifier can include at least 1, 2, 3, 4, 5, 10, 15, 20, 25, 30, 35, 40, 45, 50, 55, 60, 65, 70, 75 or more genes selected from Table 1, and at least 1, 2, 3, 4, 5, 10, 15, 20, 25, 30, 35, 40, 45, 50, 55, 60, 65, 70, 75 or more genes selected from Table 2. The total number of genes selected from Table 1 can be equal to, or different from, that selected from Table 2.

Prognostic genes or class predictors capable of distinguishing three or more outcome classes can also be employed in the present invention. These prognostic genes can be identified using multi-class correlation metrics. Suitable programs for carrying out multi-class correlation analysis include, but are not limited to, GeneCluster 2 software (MIT Center for Genome Research at Whitehead Institute, Cambridge, Mass.). Under the analysis, patients having a specified type of leukemia are divided into at least three classes, and each class of patients has a different respective clinical outcome. The prognostic genes identified under multi-class correlation analysis are differentially expressed in PBMCs of one class of patients relative to PBMCs of other classes of patients. In one embodiment, the identified prognostic genes are correlated with a class distinction at above the 1%, 5%, 10%, 25%, or 50% significance level under a permutation test. The class distinction represents an idealized expression pattern of the identified genes in peripheral blood samples of patients who have different clinical outcomes.

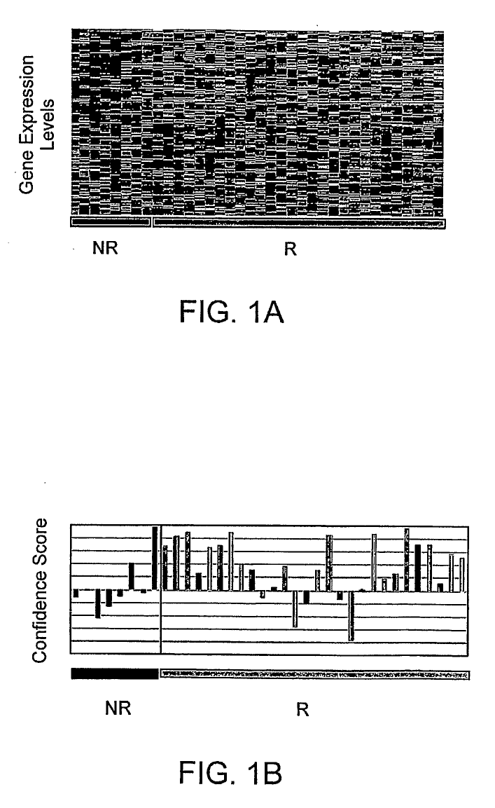

For example, FIGS. 1A and 1B illustrate the identification and cross validation of gene classifiers for distinction of PBMCs from patients who did or did not respond to Mylotarg combination therapy. FIG. 1A shows the relative expression levels of 98 class-correlated genes. As graphically presented, 49 genes were elevated in responding patient PBMCs relative to non-responding patient PBMCs and the other 49 genes were elevated in non-responding patient PBMCs relative to responding patient PBMCs. FIG. 1B demonstrates cross validation results for each sample using a class predictor consisting of the 154 genes depicted in Tables 1 and 2. A leave-one out cross validation was performed and the prediction strengths were calculated for each sample. Samples are ordered in the same order as the nearest neighbor analysis in FIG. 1A.

The 154-gene classifier exhibited a sensitivity of 82%, correctly identifying 24 of the 28 true responders in the study. The gene classifier also exhibited a specificity of 75%, correctly identifying 6 of the 8 true non-responders in the study. Similar sensitivities, specificities and overall accuracies were observed with optimal gene classifiers identified by 10-fold and leave-one-out cross validation approaches.

The above investigation evaluated expression patterns in peripheral blood samples of AML patients prior to therapy and identified transcriptional signatures correlated with initial response to therapy. The result of this study demonstrates that pharmacogenomic peripheral blood profiling strategies enable identification of patients with high likelihoods of positive or negative outcomes in response to GO combination therapy.

### Diagnosis or Monitoring the Development, Progression or Treatment of AML

The above described methods, including preparation of blood samples, assembly of class predictors, and construction and comparison of expression profiles, can be readily adapted for the diagnosis or monitoring the development, progression or treatment of AML. This can be achieved by comparing the expression profile of one or more AML disease genes in a subject of interest to at least one reference expression profile of the AML disease gene(s). The reference expression profile(s) can include an average expression profile, or a set of individual expression profiles each of which represents the peripheral blood gene expression of the AML disease gene(s) in a particular AML patient or disease-free human. Similarity between the expression profile of the subject of interest and the reference expression profile(s) is indicative of the presence or absence or the disease state of AML. In many embodiments, the disease genes employed for AML diagnosis are selected from Table 7.

One or more AML disease genes selected from Table 7 can be used for AML diagnosis or disease monitoring. In one embodiment, each AML disease gene has a p-value of less than 0.01, 0.005, 0.001, 0.0005, 0.0001, or less. In another embodiment, the AML disease genes comprise at least one gene having an “AML/Disease-Free” ratio of no less than 2 and at least one gene having an “AML/Disease-Free” ratio of no more than 0.5.

The leukemia disease genes of the present invention can be used alone, or in combination with other clinical tests, for leukemia diagnosis or disease monitoring. Conventional methods for detecting or diagnosing leukemia include, but are not limited to, bone marrow aspiration, bone marrow biopsy, blood tests for abnormal levels of white blood cells, platelets or hemoglobin, cytogenetics, spinal tap, chest X-ray, or physical exam for swelling of the lymph nodes, spleen and liver. Any of these methods, as well as any other conventional or nonconventional method, can be used, in addition to the methods of the present invention, to improve the accuracy of leukemia diagnosis.

The present invention also features electronic systems useful for the prognosis, diagnosis or selection of treatment of AML or other leukemias. These systems include an input or communication device for receiving the expression profile of a patient of interest or the reference expression profile(s). The reference expression profile(s) can be stored in a database or other media. The comparison between expression profiles can be conducted electronically, such as through a processor or a computer. The processor or computer can execute one or more programs which compare the expression profile of the patient of interest to the reference expression profile(s). The programs can be stored in a memory or downloaded from another source, such as an internet server. In one example, the programs include a k-nearest-neighbors or weighted voting algorithm. In another example, the electronic system is coupled to a nucleic acid array and can receive or process expression data generated by the nucleic acid array.

### Kits for Prognosis, Diagnosis or Selection of Treatment of Leukemia

In addition, the present invention features kits useful for the prognosis, diagnosis or selection of treatment of AML or other leukemias. Each kit includes or consists essentially of at least one probe for a leukemia prognosis or disease gene (e.g., a gene selected from Tables 1, 2, 3, 4, 5, 6, 7, 8 or 9). Reagents or buffers that facilitate the use of the kit can also be included. Any type of probe can be using in the present invention, such as hybridization probes, amplification primers, or antibodies.

In one embodiment, a kit of the present invention includes or consists essentially of at least 1, 2, 3, 4, 5, 6, 7, 8, 9, 10, or more polynucleotide probes or primers. Each probe/primer can hybridize under stringent conditions or nucleic acid array hybridization conditions to a different respective leukemia prognosis or disease gene. As used herein, a polynucleotide can hybridize to a gene if the polynucleotide can hybridize to an RNA transcript, or the complement thereof, of the gene. In another embodiment, a kit of the present invention includes one or more antibodies, each of which is capable of binding to a polypeptide encoded by a different respective leukemia prognosis or disease gene.

In one example, a kit of the present invention includes or consists essentially of probes (e.g., hybridization or PCR amplification probes or antibodies) for at least 1, 2, 3, 4, 5, 10, 15, 20, 25, 30, 35, 40, 45, 50, 55, 60, 65, 70, 75 or more genes selected from Table 2a, and probes for at least 1, 2, 3, 4, 5, 10, 15, 20, 25, 30, 35, 40, 45, 50, 55, 60, 65, 70, 75 or more genes selected from Table 2b. The total number of probes for the genes selected from Table 2a can be identical to, or different from, that for the genes selected from Table 2b.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The kits of the present invention can also have containers containing buffer(s) or reporter means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells. The kits of the present invention may also contain one or more controls, each representing a reference expression level of a prognostic or diagnostic gene detectable by one or more probes contained in the kits.

The present invention also allows for personalized treatment of AML or other leukemias. Numerous treatment options or regimes can be analyzed according to the present invention to identify prognostic genes for each treatment regime. The peripheral blood expression profiles of these prognostic genes in a patient of interest are indicative of the clinical outcome of the patient and, therefore, can be used for the selection of treatments that have favorable prognoses for the patient. As used herein, a “favorable” prognosis is a prognosis that is better than the prognoses of the majority of all other available treatments for the patient of interest. The treatment regime with the best prognosis can also be identified.

Treatment selection can be conducted manually or electronically. Reference expression profiles or gene classifiers can be stored in a database. Programs capable of performing algorithms such as the k-nearest-neighbors or weighted voting algorithms can be used to compare the peripheral blood expression profile of a patient of interest to the database to determine which treatment should be used for the patient.

It should be understood that the above-described embodiments and the following examples are given by way of illustration, not limitation. Various changes and modifications within the scope of the present invention will become apparent to those skilled in the art from the present description.

### EXAMPLES

### Example 1

### Clinical Trial and Data Collection

### Experimental Design

AML patients (13 females and 23 males) were exclusively of Caucasian descent and had a median age of 45 years (range of 19-66 years). Inclusion criteria for AML patients included blasts in excess of 20% in the bone marrow, morphologic diagnosis of AML according to the FAB classification system and flow cytometry analysis indicating positive CD33+ status. Participation in the clinical trial required concordant pathological diagnosis of AML by both an onsite pathologist following histological evaluation of bone marrow aspirates. A summary of the cytogenetic characteristics of the patients is presented in Table 11.

TABLE 11
Cytogenetic characteristics of PG consented AML patients
contributing baseline samples in 0903B1-206-US.

| | PG Consented |
|---|---|
| Cytogenetic Characteristic(s) | (n = 36)* |
| Normal karyotype | 12 (33%) |
| Complex karyotype (>3 abnormalities) | 6 (17%) |
| Other | 6 (17%) |
| +8 | 4 (11%) |
| not determined | 3 (8%) |
| −7 | 3 (8%) |
| inv (16) | 3 (8%) |
| −5q | 2 (6%) |
| −7q | 1 (3%) |
| −5q | 1 (3%) |
| t (11; 17) | 1 (3%) |
| +11 | 1 (3%) |
| 11q23 aberration | 1 (3%) |

All patients received the following standard course of induction chemotherapy and were then evaluated at 36 days. On Days 1 through 7, patients received continuous infusion cytarabine at 100 mg/m²/day. Daunorubicin was given intravenously (IV bolus) on Days 1 through 3 at 45 mg/m². On Day 4, gemtuzumab ozogamicin (6 mg/m²) was administered over approximately 2 hours as an IV infusion.

### Purification and Storage of PBMCs

All disease-free and AML peripheral blood samples were shipped overnight and processed to PBMCs by a Ficoll-gradient purification. Cell counts in whole blood and in the isolated PBMC pellets were measured by hematology analyzers and isolated PBMCs were stored at −80° C. until the RNA was extracted from these samples.

### RNA Extraction

RNA extraction was performed according to a modified RNeasy mini kit method (Qiagen, Valencia, Calif., USA). Briefly, PBMC pellets were digested in RLT lysis buffer containing 0.1% beta-mercaptoethanol and processed for total RNA isolation using the RNeasy mini kit. A phenol:chloroform extraction was then performed, and the RNA was repurified using the Rneasy mini kit reagents. Eluted RNA was quantified using a Spectramax 96 well plate UV reader (Molecular Devices, Sunnyvale, Calif., USA) monitoring A260/280 OD values. The quality of each RNA sample was assessed by gel electrophoresis.

### RNA Amplification and Generation of GeneChip Hybridization Probe

Labeled targets for oligonucleotide arrays were prepared according to a standard laboratory method. In brief, two micrograms of total RNA were converted to cDNA using an oligo-(dT)24 primer containing a T7 DNA polymerase promoter at the 5′ end. The cDNA was used as the template for in vitro transcription using a T7 DNA polymerase kit (Ambion, Woodlands, Tex., USA) and biotinylated CTP and UTP (Enzo, Farmingdale, N.Y., USA). Labeled cRNA was fragmented in 40 mM Tris-acetate pH 8.0, 100 mM KOAc, 30 mM MgOAc for 35 min at 94° C. in a final volume of 40 mL. Ten micrograms of labeled target were diluted in 1×MES buffer with 100 mg/mL herring sperm DNA and 50 mg/mL acetylated BSA. In vitro synthesized transcripts of 11 bacterial genes were included in each hybridization reaction. The abundance of these transcripts ranged from 1:300000 (3 ppm) to 1:1000 (1000 ppm) stated in terms of the number of control transcripts per total transcripts. Labeled probes were denatured at 99° C. for 5 min and then 45° C. for 5 min and hybridized to HG_U133A oligonucleotide arrays comprised of over 22000 human genes (Affymetrix, Santa Clara, Calif., USA) according to the Affymetrix GeneChip Analysis Suite User Guide (Affymetrix). Arrays were hybridized for 16 h at 45° C. with rotation at 60 rpm. After hybridization, the hybridization mixtures were removed and stored, and the arrays were washed and stained with streptavidin R-phycoerythrin (Molecular Probes) using the GeneChip Fluidics Station 400 (Affymetrix) and scanned with an HP GeneArray Scanner (Hewlett Packard, Palo Alto, Calif., USA) following the manufacturer's instructions. These hybridization and wash conditions are collectively referred to as “nucleic acid array hybridization conditions.”

### Generation of Affymetrix Signals

Array images were processed using the Affymetrix MicroArray Suite (MAS5) software such that raw array image data (.dat) files produced by the array scanner were reduced to probe feature-level intensity summaries (.cel files) using the desktop version of MAS5. Using the Gene Expression Data System (GEDS) as a graphical user interface, users provided a sample description to the Expression Profiling Information and Knowledge System (EPIKS) Oracle database and associated the correct .cel file with the description. The database processes then invoked the MAS5 software to create probeset summary values; probe intensities were summarized for each sequence using the Affymetrix Affy Signal algorithm and the Affymetrix Absolute Detection metric (Absent, Present, or Marginal) for each probeset. MAS5 was also used for the first pass normalization by scaling the trimmed mean to a value of 100. The “average difference” values for each transcript were normalized to “frequency” values using the scaled frequency normalization method (Hill, et al., Genome Biol., 2(12):research0055.1-0055.13 (2001)) in which the average differences for 11 control cRNAs with known abundance spiked into each hybridization solution were used to generate a global calibration curve. This calibration was then used to convert average difference values for all transcripts to frequency estimates, stated in units of parts per million ranging from 1:300,000 (3 parts per million (ppm)) to 1:1000 (1000 ppm) The database processes also calculated a series of chip quality control metrics and stored all the raw data and quality control calculations in the database. Only hybridized samples passing QC criteria were included in the analysis.

### Example 2

### Disease-Associated Transcripts in AML PBMCs

U133A-derived transcriptional profiles of the 36 AML PBMC samples were co-normalized using the scaled frequency normalization method with 20 MDS PBMC and 45 healthy volunteer PBMC. A total of 7879 transcripts were detected in one or more profiles with a maximal frequency greater than or equal to 10 ppm (denoted as 1 P, 1≧10 ppm) across the profiles.

To identify AML-associated transcripts, average fold differences between AML and normal PBMCs were calculated by dividing the mean level of expression in the AML profiles by the mean level of expression in normal profiles. A Student's t-test (two-sample, unequal variance) was used to assess the significance of the difference in expression between the groups.

For unsupervised hierarchical clustering, the 7879 transcripts meeting the expression filter 1P, 1≧10 ppm were used. Data were log transformed and gene expression values were median centered, and profiles were clustered using an average linkage clustering approach with an uncentered correlation similarity metric.

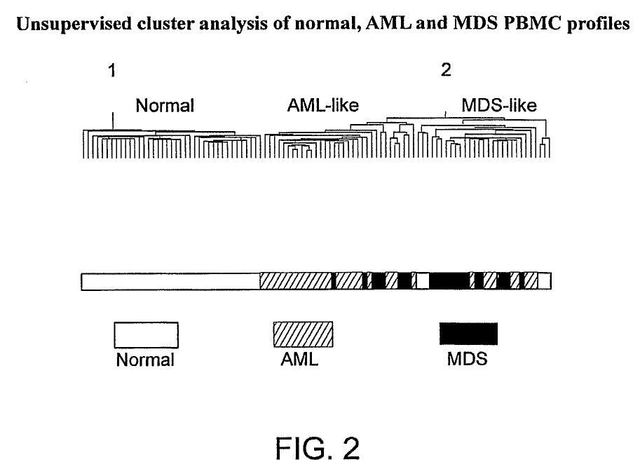

Unsupervised analysis using hierarchical clustering demonstrated that PBMCs from AML, MDS and normal healthy individuals clustered into two main clusters, with the first subgroup composed exclusively of normal PBMCs and a second subgroup composed of AML, MDS and normal PBMCs (FIG. 2). The second subgroup broke further into two distinguishable subclusters composed of an AML-like cluster populated mainly with AML PBMC profiles, an MDS-like cluster populated mainly with MDS PBMC profiles.

AML-associated transcripts in peripheral blood were identified by comparing mean levels of expression in PBMCs from the group of healthy volunteers (n=45) with mean levels of expression in PBMCs from the AML patients (n=36). The numbers of transcripts exhibiting at least a 2-fold average difference between normal and AML PBMCs at increasing levels of significance are presented in Table 12. A total of 660 transcripts possessed at least an average 2-fold difference between the AML profiles and normal PBMC profiles and a significance in an unpaired Student's t-test less than 0.001. These transcripts are presented in Table 7, above. Of these, 382 transcripts exhibited a mean elevated level of expression 2 fold or higher in AML and the fifty genes with the greatest fold elevation are presented in Table 8. A total of 278 transcripts exhibited a mean reduced level of expression 2-fold or lower in AML and the fifty genes with the greatest fold reduction in AML are presented in Table 9.

TABLE 12
Numbers of two-fold changed genes between AML and
disease-free PBMCs meeting increasing levels of significance

| | No. of transcripts with average 2-fold |
|---|---|
| Significance Level | change in AML PBMCs |
| p < 1 × 10-3 | 660 |
| p < 1 × 10-4 | 575 |
| p < 1 × 10-5 | 491 |
| p < 1 × 10-6 | 407 |
| p < 1 × 10-7 | 319 |
| p < 1 × 10-8 | 264 |
| p < 1 × 10-9 | 218 |

In these studies a total of 382 transcripts possessed significantly higher levels of expression in AML PBMCs. Elevated levels of expression may be due to 1) increased transcriptional activation in circulating PBMCs or 2) elevated levels of certain subtypes of cells in circulating PBMCs. Many of the transcripts that are elevated in AML PBMCs in this study appear to be contributed by leukemic blasts present in the peripheral circulation of these patients. Many of the transcripts are known to be specifically expressed and/or linked to disease-processes in immature or leukemic blasts (myeloperoxidase, v-myb myeloblastosis proto-oncogene, v-kit proto-oncogene, fms-related tyrosine kinase 3, CD34). In addition, many of the transcripts with the highest level of expression in AML PBMCs are at undetectable or extremely low levels in purified populations of monocytes, B-cells, T-cells, and neutrophils (data not shown) and were classified as low expressors in a healthy volunteer observational study. Thus the majority of transcripts observed to present in higher quantitites in AML PBMCs do not appear to be mainly due to transcriptional activation but rather due to the presence of leukemic blasts in the circulation of AML patients.

Conversely, disease-associated transcripts at significantly lower levels in AML PBMCs appear to be transcripts exhibiting high levels of expression in one or more of the normal types of cells typically isolated by cell-purification tubes (monocytes, B-cells, T-cells, and copurifying neutrophils). For instance, eight of the top ten transcripts at lower levels in AML PBMCs possess average levels of expression in their respective purified cell type of greater than 50 ppm, and were classified as high expressors in a healthy volunteer observational study. Thus the majority of transcripts observed to be present in lower quantities in AML PBMCs do not appear to be mainly due to transcriptional repression but rather due to the decreased presence of normal mononuclear cells in the blast-rich circulation of patients with AML.

### Example 3

### Transcriptional Effects of Therapy

A total of 27 AML patients provided evaluable baseline and Day 36 post-treatment PBMC samples. The U133A-derived transcriptional profiles of the 27 paired AML PBMC samples were co-normalized using the scaled frequency normalization method. A total of 8809 transcripts were detected in one or more profiles with a maximal frequency greater than or equal to 10 ppm (denoted as 1P, 1≧10 ppm) across the profiles.

To identify transcripts altered during the course of therapy, average fold differences between Day 0 and Day 36 PBMC profiles were calculated by dividing the mean level of expression in the baseline Day 0 profiles by the mean level of expression in the post-treatment Day 36 profiles. A Student's t-test (two-sample, unequal variance) was used to assess the significance of the difference in expression between the groups.

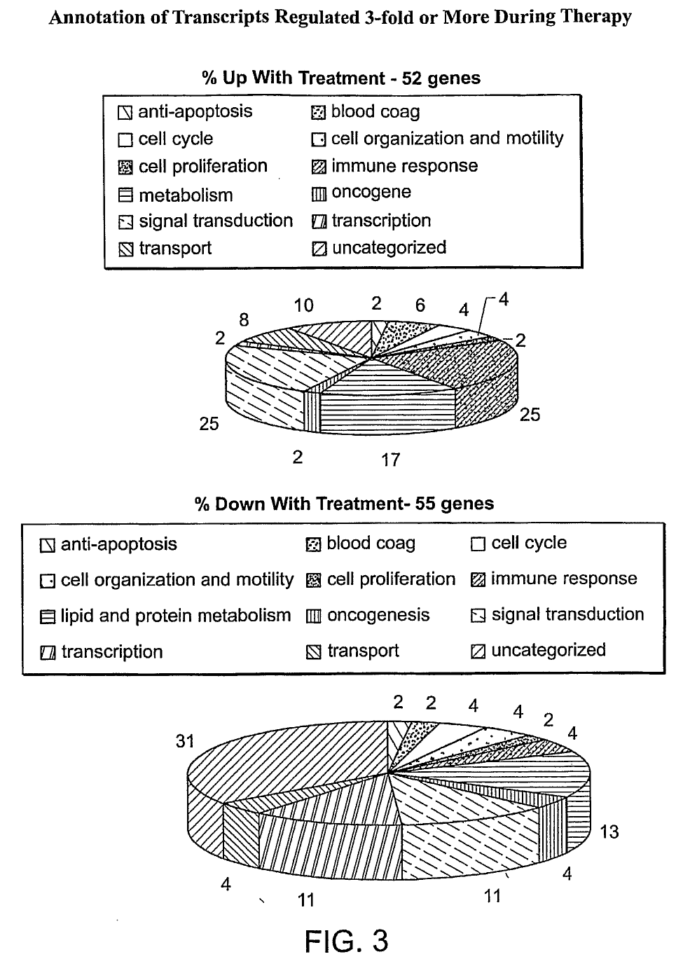

GO-based therapy-associated transcripts in peripheral blood were identified by comparing mean levels of expression in PMBCs from baseline samples (n=27) with mean levels of expression in PBMCs from the paired post-treatment samples (n=27) from the same AML patients. The numbers of transcripts exhibiting at least a 2-fold average difference between baseline and post-treatment PBMCs with increasing levels of significance are presented in Table 13. A total of 607 transcripts possessed at least an average 2-fold difference between the baseline and post-treatment samples, and significance in a paired Student's t-test of less than 0.001. Of these, 348 transcripts exhibited a mean reduced level of expression 2-fold or greater over the course of therapy and the fifty genes with the greatest fold reduction following GO therapy are presented in Table 14. A total of 259 transcripts exhibited a mean elevated level of expression 2-fold or greater over the course of therapy and the fifty genes with the greatest fold elevation following GO therapy are presented in Table 15. The genes most strongly altered over the course of therapy (mean induction or repression of 3-fold or greater) were annotated with respect to their cellular functions according to their Gene Ontology annotation and the percent of transcripts in each category are presented in FIG. 3.

TABLE 13
Numbers of two-fold changed genes between Day 0 (baseline) and
Day 36 (final visit) meeting increasing levels of significance

| | No. of transcripts with average |
|---|---|
| | 2-fold change between |
| Significance Level | baseline (Day 0) and final visit (Day 36) |
| p < 1 × 10-3 | 607 |
| p < 1 × 10-4 | 451 |
| p < 1 × 10-5 | 272 |
| p < 1 × 10-6 | 122 |
| p < 1 × 10-7 | 38 |
| p < 1 × 10-8 | 16 |
| p < 1 × 10-9 | 5 |

TABLE 14
Top 50 transcripts significantly repressed (p < 0.001)
in AML PBMCs following 36-day therapy regimen

| | | | | Fold Diff | |
|---|---|---|---|---|---|
| | | | | (Final/ | p-value |
| Affymetrix ID | Name | Cyto Band | Unigene ID | Baseline) | (unequal) |
| 205051_s_at | v-kit Hardy-Zuckerman 4 | 4q11-q12 | Hs.81665 | 0.13 | 3.02E−06 |
| | feline sarcoma viral | | | | |
| | oncogene homolog | | | | |
| 206310_at | serine protease inhibitor, | 4q11 | Hs.98243 | 0.14 | 1.06E−04 |
| | Kazal type, 2 (acrosin- | | | | |
| | trypsin inhibitor) | | | | |
| 209905_at | homeo box A9 | 7p15-p14 | Hs.127428 | 0.14 | 6.28E−04 |
| 209160_at | aldo-keto reductase | 10p15-p14 | Hs.78183 | 0.15 | 1.71E−04 |
| | family 1, member C3 (3- | | | | |
| | alpha hydroxysteroid | | | | |
| | dehydrogenase, type II) | | | | |
| 215382_x_at | tryptase beta 1, tryptase, | 16p13.3 | Hs.347933 | 0.15 | 8.80E−04 |
| | alpha | | | | |
| 204798_at | v-myb myeloblastosis | 6q22-q23 | Hs.1334 | 0.16 | 4.65E−07 |
| | viral oncogene homolog | | | | |
| | (avian) | | | | |
| 207741_x_at | tryptase, alpha | 16p13.3 | Hs.334455 | 0.16 | 7.19E−04 |
| 214651_s_at | homeo box A9 | 7p15-p14 | Hs.127428 | 0.16 | 2.12E−04 |
| 205131_x_at | stem cell growth factor; | 19q13.3 | Hs.105927 | 0.16 | 3.08E−05 |
| | lymphocyte secreted C- | | | | |
| | type lectin | | | | |
| 211709_s_at | stem cell growth factor; | 19q13.3 | Hs.105927 | 0.16 | 3.85E−06 |
| | lymphocyte secreted C- | | | | |
| | type lectin | | | | |
| 219054_at | hypothetical protein | 5p13.2 | Hs.13528 | 0.17 | 1.19E−05 |
| | FLJ14054 | | | | |
| 203948_s_at | myeloperoxidase | 17q23.1 | Hs.1817 | 0.17 | 1.36E−04 |
| 203949_at | myeloperoxidase | 17q23.1 | Hs.1817 | 0.17 | 2.81E−05 |
| 204304_s_at | prominin-like 1 (mouse) | 4p15.33 | Hs.112360 | 0.17 | 3.79E−05 |
| 201892_s_at | IMP (inosine | 3p21.2 | Hs.75432 | 0.18 | 8.66E−07 |
| | monophosphate) | | | | |
| | dehydrogenase 2 | | | | |
| 219837_s_at | cytokine-like protein C17 | 4p16-p15 | Hs.13872 | 0.18 | 5.00E−04 |
| 206674_at | fms-related tyrosine | 13q12 | Hs.385 | 0.18 | 1.01E−06 |
| | kinase 3 | | | | |
| 201416_at | Meis1, myeloid ecotropic | 17p11.2, | Hs.83484 | 0.18 | 8.38E−04 |
| | viral integration site 1 | 6p22.3 | | | |
| | homolog 3 (mouse), SRY | | | | |
| | (sex determining region | | | | |
| | Y)-box 4 | | | | |
| 221004_s_at | integral membrane | 2q37 | Hs.111577 | 0.20 | 6.77E−05 |
| | protein 3 | | | | |
| 211743_s_at | proteoglycan 2, bone | 11q12 | Hs.99962 | 0.20 | 9.21E−04 |
| | marrow (natural killer cell | | | | |
| | activator, eosinophil | | | | |
| | granule major basic | | | | |
| | protein) | | | | |
| 205609_at | angiopoietin 1 | 8q22.3-q23 | Hs.2463 | 0.21 | 3.50E−05 |
| 210783_x_at | stem cell growth factor; | 19q13.3 | Hs.105927 | 0.22 | 8.73E−05 |
| | lymphocyte secreted C- | | | | |
| | type lectin | | | | |
| 218788_s_at | hypothetical protein | 1q44 | Hs.8109 | 0.22 | 3.92E−06 |
| | FLJ21080 | | | | |
| 209790_s_at | caspase 6, apoptosis- | 4q25 | Hs.3280 | 0.23 | 2.24E−04 |
| | related cysteine protease | | | | |
| 202589_at | thymidylate synthetase | 18p11.32 | Hs.82962 | 0.24 | 3.96E−04 |
| 201418_s_at | Meis1, myeloid ecotropic | 17p11.2, | Hs.83484 | 0.24 | 7.62E−05 |
| | viral integration site 1 | 6p22.3 | | | |
| | homolog 3 (mouse), SRY | | | | |
| | (sex determining region | | | | |
| | Y)-box 4 | | | | |
| 201459_at | RuvB-like 2 (E. coli) | 19q13.3 | Hs.6455 | 0.24 | 8.40E−06 |
| 209757_s_at | v-myc myelocytomatosis | 2p24.1 | Hs.25960 | 0.25 | 1.59E−04 |
| | viral related oncogene, | | | | |
| | neuroblastoma derived | | | | |
| | (avian) | | | | |
| 213258_at | unknown | N/A | Hs.288582 | 0.25 | 1.55E−05 |
| 212115_at | hypothetical protein | 16p13.11 | Hs.172035 | 0.25 | 3.00E−04 |
| | FLJ13092 | | | | |
| 204040_at | KIAA0161 gene product | 2p25.3 | Hs.78894 | 0.26 | 4.12E−07 |
| 218858_at | hypothetical protein | 8q12.2 | Hs.87729 | 0.26 | 5.84E−04 |
| | FLJ12428 | | | | |
| 205899_at | cyclin A1 | 13q12.3-q13 | Hs.79378 | 0.26 | 4.58E−04 |
| 201310_s_at | P311 protein | 5q21.3 | Hs.142827 | 0.26 | 2.90E−06 |
| 206589_at | growth factor | 1p22 | Hs.73172 | 0.27 | 1.28E−05 |
| | independent 1 | | | | |
| 222036_s_at | MCM4 minichromosome | 8q12-q13 | Hs.154443 | 0.28 | 4.13E−04 |
| | maintenance deficient 4 | | | | |
| | (S. cerevisiae) | | | | |
| 201596_x_at | keratin 18 | 12q13 | Hs.65114 | 0.28 | 5.76E−04 |
| 201162_at | insulin-like growth factor | 4q12 | Hs.119206 | 0.28 | 2.51E−06 |
| | binding protein 7 | | | | |
| 203787_at | single-stranded DNA | 5q14.1 | Hs.169833 | 0.29 | 7.97E−05 |
| | binding protein 2 | | | | |
| 219218_at | hypothetical protein | 17q25.3 | Hs.98968 | 0.29 | 1.32E−04 |
| | FLJ23058 | | | | |
| 220416_at | KIAA1939 protein | 15q15.2 | Hs.182738 | 0.29 | 5.92E−05 |
| 201307_at | hypothetical protein | 4q13.3 | Hs.8768 | 0.29 | 1.17E−05 |
| | FLJ10849 | | | | |
| 201841_s_at | heat shock 27 kD protein 1 | 7p12.3 | Hs.76067 | 0.30 | 7.13E−04 |
| 209360_s_at | runt-related transcription | 21q22.3 | Hs.129914 | 0.30 | 1.79E−05 |
| | factor 1 (acute myeloid | | | | |
| | leukemia 1; aml1 | | | | |
| | oncogene) | | | | |
| 202502_at | acyl-Coenzyme A | 1p31 | Hs.79158 | 0.31 | 1.62E−06 |
| | dehydrogenase, C-4 to | | | | |
| | C-12 straight chain | | | | |
| 202503_s_at | KIAA0101 gene product | 15q22.1 | Hs.81892 | 0.31 | 3.51E−04 |
| 201930_at | MCM6 minichromosome | 2q21 | Hs.155462 | 0.31 | 1.36E−05 |
| | maintenance deficient 6 | | | | |
| | (MIS5 homolog, S. pombe) | | | | |
| | (S. cerevisiae) | | | | |
| 201417_at | unknown | N/A | N/A | 0.31 | 1.07E−04 |
| 202746_at | unknown | N/A | N/A | 0.32 | 6.07E−04 |
| 212009_s_at | stress-induced- | 11q13 | Hs.75612 | 0.32 | 4.03E−06 |
| | phosphoprotein 1 | | | | |
| | (Hsp70/Hsp90- | | | | |
| | organizing protein) | | | | |

TABLE 15
Top 50 transcripts significantly elevated (p < 0.001) in AML PBMCs following
36-day therapy regimen

| | | | | Fold Diff | |
|---|---|---|---|---|---|
| | | Cyto | | (Final/ | p-value |
| Affymetrix ID | Name | Band | Unigene ID | Baseline) | (unequal) |
| 201506_at | transforming growth | 5q31 | Hs.118787 | 7.89 | 9.88E−09 |
| | factor, beta-induced, | | | | |
| | 68 kD | | | | |
| 210244_at | cathelicidin antimicrobial | 3p21.3 | Hs.51120 | 7.53 | 2.43E−05 |
| | peptide | | | | |
| 203887_s_at | thrombomodulin | 20p12-cen | Hs.2030 | 6.84 | 3.15E−07 |
| 202437_s_at | cytochrome P450, | 2p21 | Hs.154654 | 6.25 | 1.56E−04 |
| | subfamily I (dioxin- | | | | |
| | inducible), polypeptide 1 | | | | |
| | (glaucoma 3, primary | | | | |
| | infantile) | | | | |
| 212531_at | lipocalin 2 (oncogene | 9q34 | Hs.204238 | 6.05 | 6.81E−05 |
| | 24p3) | | | | |
| 206343_s_at | neuregulin 1 | 8p21-p12 | Hs.172816 | 5.25 | 1.02E−06 |
| 203888_at | thrombomodulin | 20p12-cen | Hs.2030 | 5.12 | 1.46E−06 |
| 210512_s_at | vascular endothelial | 6p12 | Hs.73793 | 5.05 | 3.55E−07 |
| | growth factor | | | | |
| 202436_s_at | cytochrome P450, | 2p21 | Hs.154654 | 4.93 | 2.11E−04 |
| | subfamily I (dioxin- | | | | |
| | inducible), polypeptide 1 | | | | |
| | (glaucoma 3, primary | | | | |
| | infantile) | | | | |
| 203821_at | diphtheria toxin receptor | 5q23 | Hs.799 | 4.89 | 2.64E−07 |
| | (heparin-binding | | | | |
| | epidermal growth factor- | | | | |
| | like growth factor) | | | | |
| 206881_s_at | leukocyte | 19q13.4 | Hs.113277 | 4.76 | 2.08E−06 |
| | immunoglobulin-like | | | | |
| | receptor, subfamily A | | | | |
| | (without TM domain), | | | | |
| | member 3 | | | | |
| 205237_at | ficolin | 9q34 | Hs.252136 | 4.64 | 1.21E−08 |
| | (collagen/fibrinogen | | | | |
| | domain containing) 1 | | | | |
| 208146_s_at | carboxypeptidase, | 7p15-p14 | Hs.95594 | 4.53 | 9.53E−09 |
| | vitellogenic-like | | | | |
| 220532_s_at | LR8 protein | 7q35 | Hs.190161 | 4.51 | 6.60E−04 |
| 38037_at | diphtheria toxin receptor | 5q23 | Hs.799 | 4.36 | 1.13E−06 |
| | (heparin-binding | | | | |
| | epidermal growth factor- | | | | |
| | like growth factor) | | | | |
| 201566_x_at | inhibitor of DNA binding | 2p25 | Hs.180919 | 4.31 | 1.15E−08 |
| | 2, dominant negative | | | | |
| | helix-loop-helix protein | | | | |
| 203435_s_at | membrane metallo- | 3q25.1-q25.2 | Hs.1298 | 4.20 | 9.64E−04 |
| | endopeptidase (neutral | | | | |
| | endopeptidase, | | | | |
| | enkephalinase, CALLA, | | | | |
| | CD10) | | | | |
| 213524_s_at | putative lymphocyte | 1q32.2-q41 | Hs.95910 | 4.17 | 7.96E−08 |
| | G0/G1 switch gene | | | | |
| 205174_s_at | glutaminyl-peptide | 2p22.3 | Hs.79033 | 4.11 | 2.91E−10 |
| | cyclotransferase | | | | |
| | (glutaminyl cyclase) | | | | |
| 204115_at | guanine nucleotide | 7q31-q32 | Hs.83381 | 4.10 | 1.06E−05 |
| | binding protein 11 | | | | |
| 221211_s_at | chromosome 21 open | 21q22.3 | Hs.41267 | 3.99 | 7.25E−06 |
| | reading frame 7 | | | | |
| 202018_s_at | lactotransferrin | 3q21-q23 | Hs.105938 | 3.98 | 2.62E−04 |
| 211924_s_at | plasminogen activator, | 19q13 | Hs.179657 | 3.86 | 2.20E−07 |
| | urokinase receptor | | | | |
| 204006_s_at | Fc fragment of IgG, low | 1q23 | Hs.372679 | 3.75 | 1.62E−04 |
| | affinity IIIa, receptor for | | | | |
| | (CD16), Fc fragment of | | | | |
| | IgG, low affinity IIIb, | | | | |
| | receptor for (CD16) | | | | |
| 201565_s_at | inhibitor of DNA binding | 2p25 | Hs.180919 | 3.68 | 4.06E−10 |
| | 2, dominant negative | | | | |
| | helix-loop-helix protein | | | | |
| 206130_s_at | asialoglycoprotein | 17p | Hs.1259 | 3.65 | 1.56E−05 |
| | receptor 2 | | | | |
| 203979_at | cytochrome P450, | 2q33-qter | Hs.82568 | 3.57 | 3.78E−04 |
| | subfamily XXVIIA (steroid | | | | |
| | 27-hydroxylase, | | | | |
| | cerebrotendinous | | | | |
| | xanthomatosis), | | | | |
| | polypeptide 1 | | | | |
| 206390_x_at | platelet factor 4 | 4q12-q21 | Hs.81564 | 3.57 | 9.97E−06 |
| 210146_x_at | leukocyte | 19q13.4 | Hs.22405 | 3.49 | 5.04E−08 |
| | immunoglobulin-like | | | | |
| | receptor, subfamily B | | | | |
| | (with TM and ITIM | | | | |
| | domains), member 2 | | | | |
| 204112_s_at | histamine N- | 2q21.1 | Hs.81182 | 3.49 | 1.30E−06 |
| | methyltransferase | | | | |
| 211135_x_at | leukocyte | 19q13.4 | Hs.105928 | 3.49 | 4.18E−07 |
| | immunoglobulin-like | | | | |
| | receptor, subfamily B | | | | |
| | (with TM and ITIM | | | | |
| | domains), member 3 | | | | |
| 208601_s_at | tubulin, beta 1 | 20q13.32 | Hs.303023 | 3.45 | 3.68E−04 |
| 210845_s_at | plasminogen activator, | 19q13 | Hs.179657 | 3.42 | 1.72E−09 |
| | urokinase receptor | | | | |
| 211527_x_at | vascular endothelial | 6p12 | Hs.73793 | 3.40 | 1.08E−05 |
| | growth factor | | | | |
| 221210_s_at | chromosome 1 open | 1q25 | Hs.23756 | 3.40 | 2.18E−07 |
| | reading frame 13 | | | | |
| 201393_s_at | insulin-like growth factor | 6q26 | Hs.76473 | 3.40 | 1.75E−06 |
| | 2 receptor | | | | |
| 205568_at | aquaporin 9 | 15q22.1-22.2 | Hs.104624 | 3.33 | 3.73E−05 |
| 221698_s_at | C-type (calcium | 12p13.2-p12.3 | Hs.161786 | 3.33 | 1.08E−06 |
| | dependent, | | | | |
| | carbohydrate-recognition | | | | |
| | domain) lectin, | | | | |
| | superfamily member 12 | | | | |
| 204081_at | neurogranin (protein | 11q24 | Hs.26944 | 3.31 | 2.29E−05 |
| | kinase C substrate, RC3) | | | | |
| 206359_at | suppressor of cytokine | 17q25.3 | Hs.345728 | 3.28 | 1.70E−07 |
| | signaling 3 | | | | |
| 219593_at | peptide transporter 3 | 11q13.1 | Hs.237856 | 3.27 | 6.44E−07 |
| 204007_at | Fc fragment of IgG, low | 1q23 | Hs.176663 | 3.26 | 3.24E−04 |
| | affinity IIIa, receptor for | | | | |
| | (CD16) | | | | |
| 201739_at | serum/glucocorticoid | 6q23 | Hs.296323 | 3.21 | 9.28E−08 |
| | regulated kinase | | | | |
| 203645_s_at | CD163 antigen | 12p13.3 | Hs.74076 | 3.20 | 3.41E−04 |
| 203414_at | monocyte to macrophage | 17q | Hs.79889 | 3.16 | 5.41E−09 |
| | differentiation-associated | | | | |
| 214696_at | hypothetical protein | 17p13.3 | Hs.29206 | 3.16 | 4.12E−08 |
| | MGC14376 | | | | |
| 210225_x_at | leukocyte | 19q13.4 | Hs.105928 | 3.13 | 1.37E−06 |
| | immunoglobulin-like | | | | |
| | receptor, subfamily B | | | | |
| | (with TM and ITIM | | | | |
| | domains), member 3 | | | | |
| 203561_at | Fc fragment of IgG, low | 1q23 | Hs.78864 | 3.11 | 1.83E−06 |
| | affinity IIa, receptor for | | | | |
| | (CD32) | | | | |
| 218454_at | hypothetical protein | 12p13.31 | Hs.178470 | 3.10 | 1.67E−07 |
| | FLJ22662 | | | | |
| 221724_s_at | C-type (calcium | 12p13 | Hs.115515 | 3.08 | 1.10E−08 |
| | dependent, | | | | |
| | carbohydrate-recognition | | | | |
| | domain) lectin, | | | | |
| | superfamily member 6 | | | | |

Comparison of pre- and post-treatment PBMC profiles from AML patients revealed a large number of differences in transcript levels over the course of therapy. Annotation of the genes apparently repressed over the course of therapy using Gene Ontology annotation (see FIG. 3) demonstrated that many of the transcripts at lower levels following therapy fell into an uncharacterized category. Further evaluation revealed that the vast majority of these transcripts were disease associated and were present at lower quantities in post-treatment samples due to the disappearance of leukemic blasts in these patients following therapy. Consistent with this observation, forty-five of the top 50 transcripts down-regulated following the GO regimen were disease (blast)-associated genes. Thus the down-regulation of v-kit, tryptase, aldo-keto reductase 1C3, homeobox A9, meis1, myeloperoxidase, and the majority of other transcripts exhibiting the greatest fold reduction appear to be due to the disappearance of leukemic blasts in the circulation, rather than direct transcriptional effects of the chemotherapy regimen.

Evaluation of the transcripts in PBMCs at higher levels following therapy revealed the opposite trend and showed that the vast majority of these transcripts were associated with normal PBMC expression and were present at higher quantities in post-treatment samples due to the reappearance of normal mononuclear cells in the majority of treated patients. A total of thirty-one of the top 50 transcripts up-regulated following the GO regimen were transcripts associated with normal mononuclear cell expression. Thus the up-regulation of the TGF-beta induced protein (68 kDa), thrombomodulin, putative lymphocyte G0/G1 switch gene, and the majority of other transcripts are likely due to the disappearance of leukemic blasts and repopulation of normal cells in the circulation, rather than direct transcriptional effects of the chemotherapy regimen.

For a smaller number of genes, transcriptional activation or repression may be the cause for differences in transcript levels. For instance, cytochrome P4501A1 (CYP1A1) is induced following therapy but is not significantly associated with normal mononuclear cell expression (i.e., CYP1A1 was not significantly repressed in AML PBMCs compared to normal PBMCs). CYP1A1 is involved in the metabolism of daunorubicin, and daunorubicin is a mechanism-based inactivator of CYP1A1 activity. Thus the elevation of CYP1A1mRNA may represent a feedback transcriptional response to the present therapeutic regimen. Interferon-inducible proteins were also elevated during the course of therapy (interferon-inducible protein 30, interferon-induced transmembrane protein 2), and these effects may also represent transcriptional inductions of interferon-dependent signaling pathways activated during the course of therapy.

Whether due to disappearance of blasts, elevations in normal cell counts or actual transcriptional activation or repression, alterations in several of the PBMC transcripts may have functional consequences on the progression of AML. TGF-beta induces cell cycle arrest and antagonizes FLT3-induced proliferation of leukemic cells, and a TGF-beta induced protein was the most strongly upregulated transcript (>7 fold elevated) in PBMCs during the course of therapy.

### Example 4

### Pretreatment Expression Patterns Associated with Veno-Occlusive Disease

U133A-derived transcriptional profiles of the 36 AML PBMC samples were co-normalized using the scaled frequency normalization method. A total of 7405 transcripts were detected in one or more profiles with a maximal frequency greater than or equal to 10 ppm (denoted as 1P, 1≧10 ppm) across the profiles.

Veno-occlusive disease (VOD) is one of the most serious complications following hematopoietic stem cell transplantation and is associated with a very high mortality in its severe form. To identify transcripts with significant differences in expression at baseline between the four patients who eventually experienced VOD and the thirty-two non-VOD patients, average fold differences between VOD and non-VOD patient profiles were calculated by dividing the mean level of expression in the four baseline VOD profiles by the mean level of expression in the 32 baseline non-VOD profiles. A Student's t-test (two-sample, unequal variance) was used to assess the significance of the difference in expression between the groups.

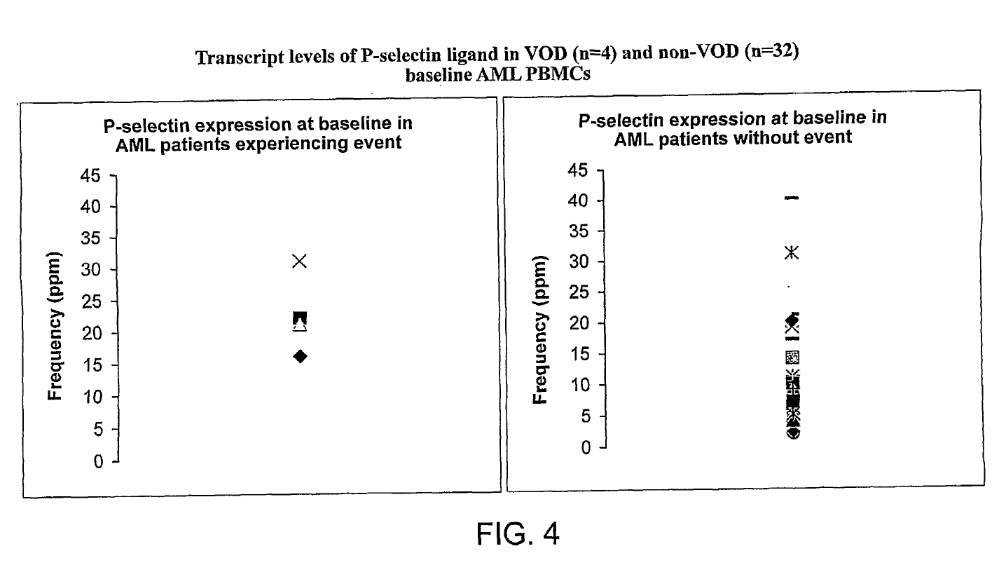

Transcripts in baseline PBMCs significantly associated with the onset of VOD were identified by comparing mean levels of expression in PMBCs from the VOD baseline samples (n=4) with mean levels of expression in PBMCs from the non-VOD baseline samples (n=32). The numbers of transcripts exhibiting at least a 2-fold average difference between VOD and non-VOD baseline PBMCs with increasing levels of significance are presented in Table 16. A total of 161 transcripts possessed at least an average 2-fold difference between the baseline VOD and non-VOD samples, and significance in a paired Student's t-test of less than 0.05. Of the 161 transcripts, only 3 transcripts exhibited a mean elevated level of expression 2-fold or greater in VOD PBMCs at baseline. These and forty-seven other transcripts showing less than 2-fold but exhibiting the greatest fold elevation in VOD patients at baseline are presented in Table 5. The levels of p-selectin ligand, a potentially biologically relevant transcript that appeared to be significantly elevated in PBMCs of patients who eventually experienced VOD, are presented in FIG. 4.

TABLE 16
Numbers of two-fold changed genes between baseline samples
of VOD patients (n = 4) and non-VOD patients (n = 32)
meeting increasing levels of significance

| | No. of transcripts with average 2-fold change |
|---|---|
| Significance Level | between baseline (Day 0) and final visit (Day 36) |
| p < 0.05 | 161 |
| p < 0.01 | 98 |
| p < 1 × 10-3 | 42 |
| p < 1 × 10-4 | 10 |
| p < 1 × 10-5 | 4 |
| p < 1 × 10-6 | 2 |

The remaining 158 transcripts exhibited a mean reduced level of expression 2-fold or greater in VOD PBMCs at baseline, and the fifty genes with the greatest fold reduction in VOD patient PBMCs at baseline are presented in Table 6. Evaluation of this set of transcripts revealed a majority of leukemic blast-associated markers. This unanticipated finding by microarray analysis actually suggests that patients with lower peripheral blast counts may be more susceptible to VOD in the context of GO-based therapy.

### Example 5

### Pretreatment Transcriptional Patterns Associated with Clinical Response

As in the preceding Example, 7405 transcripts detected with a maximal frequency greater than or equal to 10 ppm in one or more profiles were selected for further evaluation.

To identify transcripts with significant differences in expression at baseline between the 8 patients who were non-responders (NR) and the 28 patients who were responders (R), average fold differences between NR and R patient profiles were calculated by dividing the mean level of expression in the eight baseline NR profiles by the mean level of expression in the 28 baseline R profiles. A Student's t-test (two-sample, unequal variance) was used to assess the significance of the difference in expression between the groups. The numbers of transcripts exhibiting at least a 2-fold average difference between R and NR baseline PBMCs with increasing levels of significance are presented in Table 17. A total of 113 transcripts possessed at least an average 2-fold difference between the baseline R and NR samples, and significance in a paired Student's t-test of less than 0.05. Of the 113 transcripts, 6 transcripts exhibited a mean elevated level of expression 2-fold or higher in non-responder PBMCs at baseline. These and forty-four other transcripts showing less than 2-fold but exhibiting the greatest fold elevation in responding patients at baseline are presented in Table 3. A total of 107 transcripts exhibited a mean reduced level of expression 2-fold or greater in non-responder PBMCs at baseline, and the fifty genes with the greatest fold reduction are presented in Table 4.

TABLE 17
Numbers of two-fold changed genes between baseline
samples of non-responding patients (n = 8) and responding
patients (n = 28) meeting increasing levels of significance

| | No. of transcripts with average 2-fold |
|---|---|
| | change between NR and R at |
| Significance Level | baseline |
| p < 0.05 | 113 |
| p < 0.01 | 45 |
| p < 1 × 10-3 | 7 |
| p < 1 × 10-4 | 1 |

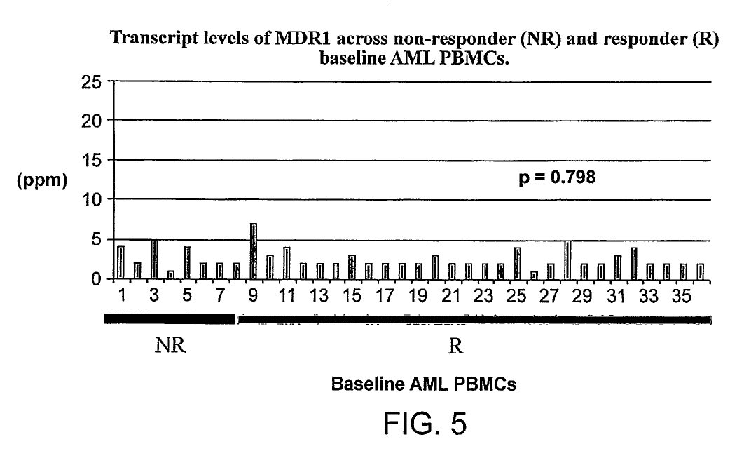

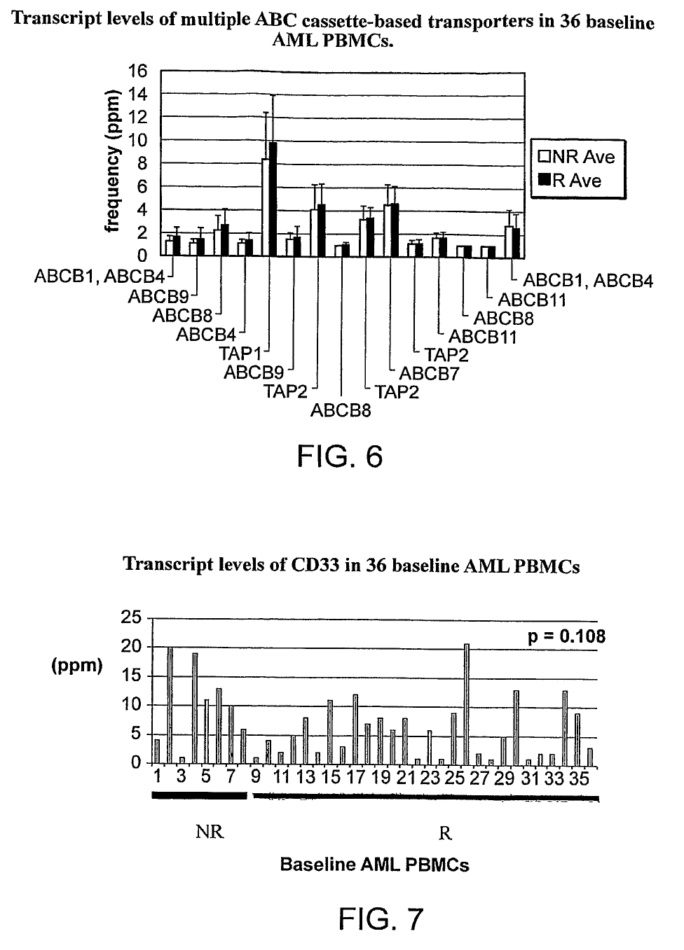

Pretreatment levels of transcripts encoded by genes with potential roles in the metabolism or mechanism of action of GO were specifically interrogated as well. Levels of the MDR1 drug efflux transporter were low in all PBMC samples and were not significantly distinct between responders and non-responders at baseline (FIG. 5). The remaining members of the ABC transporter family contained on the Affymetrix U133A gene chip were also interrogated in the event that another ABC transporter might be differentially expressed, but none of the ABC transporters were significantly distinct between responder and non-responder PBMCs at baseline (FIG. 6). Levels of transcripts encoding the CD33 cell surface receptor were detected at generally higher levels in the AML PBMCs, but like MDR1, the CD33 transcript was also not significantly distinct between R and NR PBMCs at baseline (FIG. 7).

To identify a gene classifier capable of classifying responder and non-responders on the basis of baseline gene expression patterns, gene selection and supervised class prediction were performed using Genecluster version 2.0 previously described and available at (http://www.genome.wi.mit.edu/cancer/software/genecluster2.html). For nearest neighbor analysis, expression profiles for 36 baseline AML PMBCs from were co-normalized using the scale frequency method with 14 baseline AML PBMCs from an independent clinical trial of GO in combination with daunorubicin. All expression data were z-score normalized prior to analysis. A total of 11382 sequences were used in this analysis, based on inclusion of all transcripts with frequencies possessing at least one value of greater than or equal to 5 ppm across the baseline profiles. The 36 PBMC baseline profiles from were treated as a training set, and models containing increasing numbers of features (transcript sequences) were built using a one versus all approach with a S2N similarity metric that used median values for the class estimate. All comparisons were binary distinctions, and each model (with increasing numbers of features) was evaluated in the 36 PBMC profiles by 10-fold cross validation. The optimally predictive model arising from the 10-fold cross validation of the 36 PBMC profiles was then applied to the 14 co-normalized profiles from the other clinical trial to evaluate the gene classifiers accuracy in an independent set of clinical samples taken from AML patients prior to therapy.

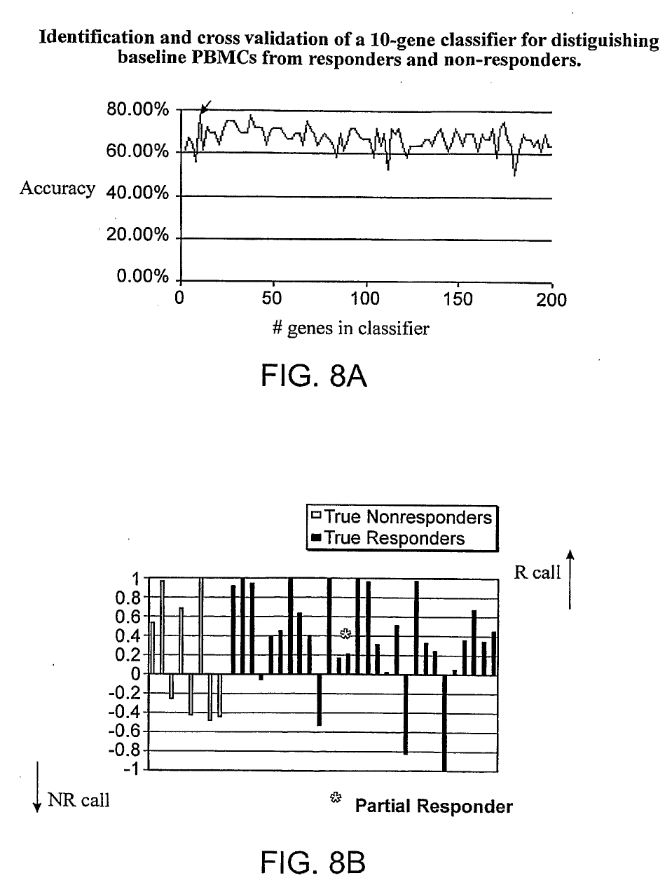

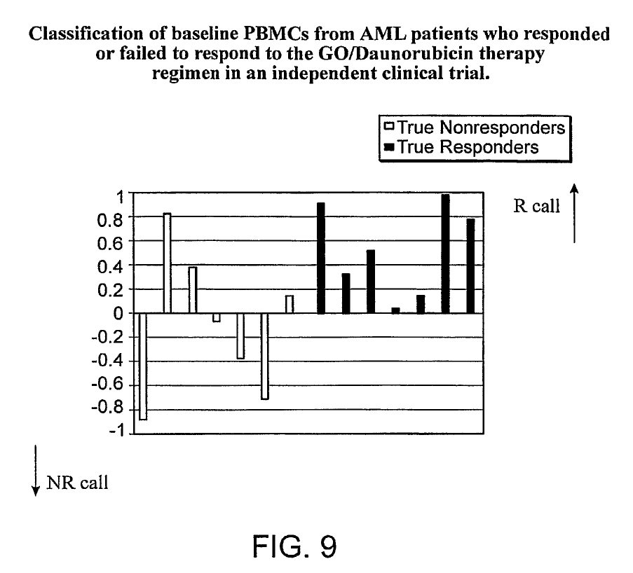

A 10-gene classifier was found to yield the highest overall prediction accuracy (78%) by 10-fold cross validation on the peripheral blood AML profiles in the present study (FIG. 8 and Table 18). This gene classifier exhibited a sensitivity of 86%, a specificity of 50%, a positive predictive value of 86% and a negative predictive value of 50%. This classifier was also applied to the 14 untested profiles from the independent study in which GO plus daunorubicin composed the therapy regimen; the results are presented in FIG. 9. For those 14 profiles, the ten gene classifier demonstrated an overall prediction accuracy of 78%, a sensitivity of 100%, a specificity of 57%, a positive predictive value of 70% and a negative predictive value of 100%.

TABLE 18
Transcripts in the 10-gene classifier associated with elevated PBMC levels in
responders (top panel) or non-responders (bottom panel) prior to therapy.

| Top S2N | | | | | |
|---|---|---|---|---|---|
| Transcripts | | Affymetrix | | | |
| Elevated in: | Rank | ID | Name | Cyto Band | Unigene ID |
| R | 1 | 203739_at | zinc finger protein 217 | 20q13.2 | Hs.155040 |
| R | 2 | 219593_at | peptide transporter 3 | 11q13.1 | Hs.237856 |
| R | 3 | 204132_s_at | forkhead box O3A | 6q21 | Hs.14845 |
| R | 4 | 210972_x_at | T cell receptor alpha | 14q11.2 | Hs.74647 |
| | | | locus | | |
| R | 5 | 205220_at | putative chemokine | 12q24.31 | Hs.137555 |
| | | | receptor; GTP-binding | | |
| | | | protein | | |
| NR | 1 | 208581_x_at | metallothionein 1L, | 16q13 | Hs.278462 |
| | | | metallothionein 1X | | |
| NR | 2 | 208963_x_at | fatty acid desaturase 1 | 11q12.2-q13.1 | Hs.132898 |
| NR | 3 | 216336_x_at | uncharacterized | n/a | n/a |
| NR | 4 | 209407_s_at | deformed epidermal | 11p15.5 | Hs.6574 |
| | | | autoregulatory factor 1 | | |
| | | | (Drosophila) | | |
| NR | 5 | 203725_at | growth arrest and DNA- | 1p31.2-p31.1 | Hs.80409 |
| | | | damage-inducible, alpha | | |

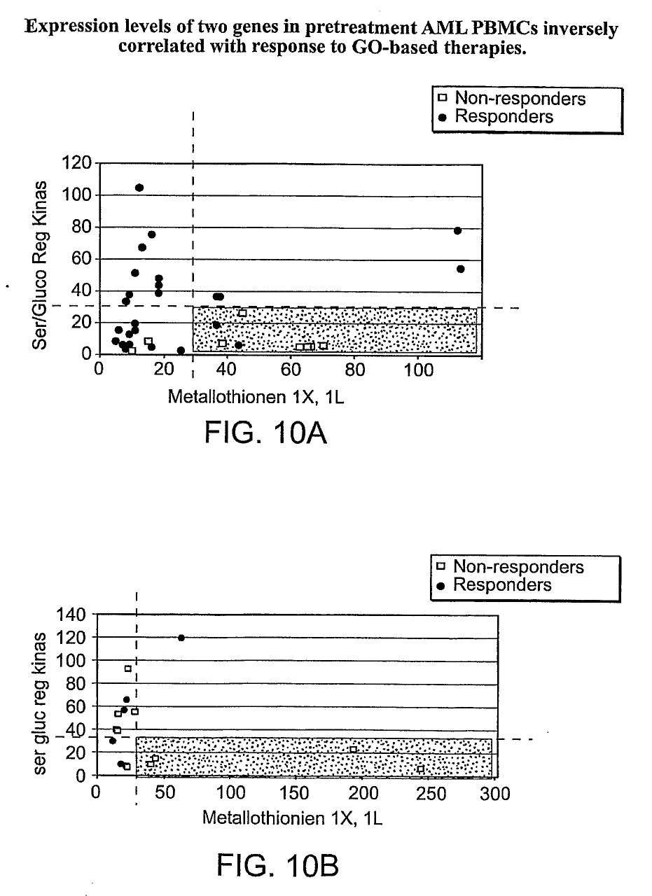

Some pharmacogenomic co-diagnostics developed in the future will likely rely on qRT-PCR based assays that can utilize small (pair-wise or greater) combinations of genes that enable accurate classification. To identify a smaller classifier the Affymetrix-based expression levels of two genes (Table 19), metallothionein 1X/1L and serum glucocorticoid regulated kinase, which were overexpressed in AML PBMCs from non-responders and responders respectively, were plotted to determine whether a pair-wise combination of transcripts could enable classification (FIG. 10, panel A). The two gene classifier employing metallothionein 1X/1L and serum glucocorticoid regulated kinase was selected on the basis of their 1) significantly elevated or repressed fold differences between responder and non-responder categories, respectively; and 2) known annotation. The individual expression values (in terms of ppm) of each transcript in each baseline AML sample were plotted to identify cutoffs for expression that gave the highest sensitivity and specificity for class assignment. From the original 36 patients, six of the eight non-responders had serum glucocorticoid regulated kinase levels <30 ppm and metallothionein 1X/1L levels>30 ppm. Only 2 of the 28 responders possessed similar levels of gene expression. For these 36 sample, the 2-gene classifier therefore exhibited an apparent 88% overall accuracy, a sensitivity of 93%, a specificity of 75%, a positive predictive value of 93% and a negative predictive value of 75%.

### Table 19. Transcripts in the 2-Gene Classifier Associated with Elevated Levels in Responders (Serum/Gluclocorticoid Regulated Kinase) or Non-Responders (metallothionein 1L,1X) Prior to Therapy

TABLE 19
Transcripts in the 2-gene classifier associated with elevated levels
in responders (serum/gluclocorticoid regulated kinase) or non-
responders (metallothionein 1L, 1X) prior to therapy.

| | | Cyto | Unigene |
|---|---|---|---|
| Affymetrix ID | Name | Band | ID |
| 201739_at | serum/glucocorticoid | 6q23 | Hs•296323 |
| | regulated kinase | | |
| 208581_x_at | metallothionein 1L, | 16q13 | Hs•278462 |
| | metallothionein 1X | | |

This 2-gene classifier (serum glucocorticoid regulated kinase <30 ppm, metallothionein 1X,1L>30 ppm) was also applied to the 14 untested profiles from the independent clinical trial in which GO plus daunorubicin composed the therapy regimen (FIG. 10, panel B). In that study, the 2-gene classifier demonstrated identical overall performance as the 10-gene classifier, with an overall prediction accuracy of 78%, a sensitivity of 100%, a specificity of 57%, a positive predictive value of 70% and a negative predictive value of 100%.

Apparent performance characteristics of both the 10-gene and 2-gene classifiers for the first dataset of 36 samples and actual performance characteristics of both classifiers in the evaluation of the 14 independent samples are listed in Table 20.

TABLE 20
Performance characteristics of the 2-gene and 10-gene classifiers
by cross-validation and in a test set.

| | |
|---|---|
| 10 gene classifier | 2 gene classifier |

Cross-validation

| | | |
|---|---|---|
| Accuracy | 78% | 88% |
| Sensitivity | 86% | 93% |
| Specificity | 50% | 75% |
| Positive predictive value | 86% | 93% |
| Negative predictive value | 50% | 75% |

Test set

| | | |
|---|---|---|
| Accuracy | 78% | 78% |
| Sensitivity | 100% | 100% |
| Specificity | 57% | 57% |
| Positive predictive value | 70% | 70% |
| Negative predictive value | 100% | 100% |

In this analysis transcriptional profiling was applied to baseline peripheral blood samples to characterize transcriptional patterns that might provide insights into, or biomarkers for, AML patients' abilities to respond or fail to respond to a GO combination chemotherapy regimen. The largest percentage of patients in this study possessed a normal karyotype (33%), while other chromosomal abnormalities were relatively evenly distributed among the remaining patients. This heterogeneity of cytogenetic backgrounds allowed us to analyze the entire group of AML profiles without segregating them into karyotype-based groups, which in turn enabled us to search for transcriptional patterns that might be correlated with response to the GO combination regimen regardless of the molecular abnormalities involved in this complex disease. Despite the recent description of expression signatures associated with various chromosomal abnormalities in AML, it is clear that expression of many of the individual transcripts in the hallmark signatures are not unique to specific karyotypes. In addition, Bullinger et al. (2004) N. Engl. J. Med. 350:1605-16, importantly demonstrated in their recent study that relatively homogeneous transcriptional patterns correlated with overall survival were detectable in AML samples from patients despite their diverse cytogenetic backgrounds, and these prognostic profiles segregated samples from a test set of patients into good and poor outcome categories that possessed significant differences in overall survival.

An objective of the present study was not necessarily to identify generally prognostic profiles associated with overall survival, but rather to identify a transcriptional pattern in peripheral blood that, if validated, could allow identification of patients who would or would not benefit (i.e., achieve initial remission) from a GO combination chemotherapy regimen. Comparison of responder (i.e. remission) and non-responder profiles at baseline identified a number of transcripts significantly altered between the groups.

Transcripts present at higher levels in responding patients prior to therapy included T-cell receptor alpha locus, serum/glucocorticoid regulated kinase, aquaporin 9, forkhead box 03, IL8, TOSO (regulator of fas-induced apoptosis), IL1 receptor antagonist, p21/cip1, a specific subset of IFN-inducible transcripts, and other regulatory molecules. The list of transcripts elevated in responder peripheral blood appears to contain markers of both normal peripheral blood cells (lymphocytes, monocytes and neutrophils) and blast-specific transcripts alike. A higher percentage of pro-apoptotic related molecules were elevated in peripheral blood of patients who ultimately responded to therapy. FOX03 is a critical pro-apoptotic molecule that is inactivated during IL2-mediated T-cell survival and has recently been shown to be inactivated during FLT3-induced, PI3Kinase dependent stimulation of proliferation in myeloid cells. The finding that FOX03 is elevated in peripheral blood of AML patients that ultimately responded to GO combination therapy supports the theory that apoptotically “primed” cells will be more sensitive to the effects of GO based therapy regimens and possibly other chemotherapies as well. Levels of FOX01A are positively correlated with survival in AML patients receiving two different regimens.

A number of transcripts were also elevated in blood samples of AML patients who failed to respond to therapy. A comparison was made between transcripts associated with failure to respond to the current GO combination regimen and transcripts recently reported as predictive of poor outcome with respect to overall survival. Elevation in homeobox B6 levels in peripheral blood samples of non-responders in this study was consistent with the overexpression of multiple homeobox genes in patients with poor outcomes related to survival. Homeobox B6 is elevated during normal granulocytopoiesis and monocytopoiesis, but is normally turned off following cell maturation. Homeobox B6 was found to be dysregulated in a substantial percentage of AML samples and has been proposed to play a role in leukemogenesis.

The present analyses also identified several families of transcripts where overexpression appears to be correlated with failure to respond to the GO combination regimen and do not appear to be correlated with overall survival. Several metallothionein isoforms were elevated in peripheral blood samples of patients who failed to respond to the GO combination regimen. Based on the mechanism of action of GO, elevated antioxidant defenses would be expected to adversely impact the efficacy of the chalechiamicin-directed cytotoxic conjugate. These findings however contrast with those reported by Goasguen et al. (1996) Leuk. Lymphoma. 23(5-6):567-76, who identified metallothionein overexpression as strongly associated with complete remission in the context of the absence or presence of other drug-resistance phenotypes in patients with leukemias. Metallothionein isoform overexpression has recently been characterized as a hallmark of the t(15;17) chromosomal translocation in AML but none of the patients in the present study were characterized as possessing this cytogenetic abnormality. However, in that study metallothionein isoform overexpression was not specific to the t(15;17) translocation, occurring in several other karyotypes as well.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

We claim:

1. A method for predicting a clinical outcome in response to a treatment of a leukemia, the method comprising the steps of:
(1) measuring expression levels of one or more prognostic genes of the leukemia in a peripheral blood mononuclear cell sample derived from a patient prior to the treatment; and
(2) comparing each of the expression levels to a corresponding control level,
wherein the result of the comparison is predictive of a clinical outcome.

2. The method of claim 1, wherein the one or more prognostic genes comprise at least a first gene selected from a first class and a second gene selected from a second class, wherein the first class comprises genes having higher expression levels in peripheral blood mononuclear cells in patients predicted to have a less desirable clinical outcome in response to the treatment and the second class comprises genes having higher expression levels in peripheral blood mononuclear cells in patients predicted to have a more desirable clinical outcome in response to the treatment.

3. The method of claim 2, wherein the first gene is selected from Table 3 and the second gene is selected from Table 4.

4. The method of claim 2, wherein the first gene is selected from the group consisting of zinc finger protein 217, peptide transporter 3, forkhead box O3A, T cell receptor alpha locus and putative chemokine receptor/GTP-binding protein, and the second gene is selected from the group consisting of metallothionein, fatty acid desaturase 1, uncharacterized gene corresponding to Affymetrix ID 216336, deformed epidermal autoregulatory factor 1 and growth arrest and DNA-damage-inducible alpha.

5. The method of claim 2, wherein the first gene is serum glucocorticoid regulated kinase and the second gene is metallothionein 1X/1L.

6. The method of claim 1, wherein the clinical outcome is development of an adverse event.

7. The method of claim 6, wherein the adverse event is veno-occlusive disease.

8. The method of claim 7, wherein the one or more prognostic genes comprise one or more genes selected from Table 5 or Table 6.

9. The method of claim 8, wherein the one or more prognostic genes comprise p-selectin ligand.

10. The method of any one of the preceding claims, wherein the treatment comprises a gemtuzumab ozogamicin (GO) combination therapy.

11. The method of any one of the preceding claims, wherein the corresponding control level is a numerical threshold.

12. A method for predicting a clinical outcome of a leukemia, the method comprising the steps of:
(1) generating a gene expression profile from a peripheral blood sample of a patient having the leukemia; and
(2) comparing the gene expression profile to one or more reference expression profiles,
wherein the gene expression profile and the one or more reference expression profiles comprise expression patterns of one or more prognostic genes of the leukemia in peripheral blood mononuclear cells, and wherein the difference or similarity between the gene expression profile and the one or more reference expression profiles is indicative of the clinical outcome for the patient.

13. The method of claim 12, wherein the leukemia is acute leukemia, chronic leukemia, lymphocytic leukemia or nonlymphocytic leukemia.

14. The method of claim 13, wherein the leukemia is acute myeloid leukemia (AML).

15. The method of any one of claims 12-14, wherein the clinical outcome is measured by a response to an anti-cancer therapy.

16. The method of claim 15, wherein the anti-cancer therapy comprises administering one or more compounds selected from the group consisting of an anti-CD33 antibody, a daunorubicin, a cytarabine, a gemtuzumab ozogamicin, an anthracycline, and a pyrimidine or purine nucleotide analog.

17. The method of any one of claims 12-16, wherein the one or more prognostic genes comprise one or more genes selected from Table 3 or Table 4.

18. The method of claim 17, wherein the one or more prognostic genes comprise ten or more genes selected from Table 3 or Table 4.

19. The method of claim 18, wherein the one or more prognostic genes comprise twenty or more genes selected from Table 3 or Table 4.

20. The method of any one of claims 12-19, wherein step (2) comprises comparing the gene expression profile to the one or more reference expression profiles by a k-nearest neighbor analysis or a weighted voting algorithm.

21. The method of any one of claims 12-19, wherein the one or more reference expression profiles represent known or determinable clinical outcomes.

22. The method of any one of claims 12-19, wherein step (2) comprises comparing the gene expression profile to at least two reference expression profiles, each of which represents a different clinical outcome.

23. The method of claim 22, wherein each reference expression profile represents a different clinical outcome selected from the group consisting of remission to less than 5% blasts in response to the anti-cancer therapy; remission to no less than 5% blasts in response to the anti-cancer therapy; and non-remission in response to the anti-cancer therapy.

24. The method of any one of claims 12-19, wherein the one or more reference expression profiles comprise a reference expression profile representing a leukemia-free human.

25. The method of any one claims 12-19, wherein step (1) comprises generating the gene expression profile using a nucleic acid array.

26. The method of claim 15, wherein step (1) comprises generating the gene expression profile from the peripheral blood sample of the patient prior to the anti-cancer therapy.

27. A method for selecting a treatment for a leukemia patient, the method comprising the steps of:
(1) generating a gene expression profile from a peripheral blood sample derived from the leukemia patient;
(2) comparing the gene expression profile to a plurality of reference expression profiles, each representing a clinical outcome in response to one of a plurality of treatments; and
(3) selecting from the plurality of treatments a treatment which has a favorable clinical outcome for the leukemia patient based on the comparison in step (2),
wherein the gene expression profile and the one or more reference expression profiles comprise expression patterns of one or more prognostic genes of the leukemia in peripheral blood mononuclear cells.

28. The method of claim 27, wherein the one or more prognostic genes comprise one or more genes selected from Table 3 or Table 4.

29. The method of claim 28, wherein the one or more prognostic genes comprise ten or more genes selected from Table 3 or Table 4.

30. The method of claim 29, wherein the one or more prognostic genes comprise twenty or more genes selected from Table 3 or Table 4.

31. The method of any one of claims 27-30, wherein step (2) comprises comparing the gene expression profile to the plurality of reference expression profiles by a k-nearest neighbor analysis or a weighted voting algorithm.

32. A method for diagnosis, or monitoring the occurrence, development, progression or treatment, of a leukemia, the method comprising the steps of:
(1) generating a gene expression profile from a peripheral blood sample of a patient having the leukemia; and
(2) comparing the gene expression profile to one or more reference expression profiles,
wherein the gene expression profile and the one or more reference expression profiles comprise the expression patterns of one or more diagnostic genes of the leukemia in peripheral blood mononuclear cells, and wherein the difference or similarity between the gene expression profile and the one or more reference expression profiles is indicative of the presence, absence, occurrence, development, progression, or effectiveness of treatment of the leukemia in the patient.

33. The method of claim 32, wherein the leukemia is AML.

34. The method of claim 33, wherein the one or more diagnostic genes comprise one or more genes selected from Table 7.

35. The method of claim 33, wherein the one or more diagnostic genes comprise one or more genes selected from Table 8 or Table 9.

36. The method of claim 33, wherein the one or more diagnostic genes comprise ten or more genes selected from Table 7.

37. The method of claim 33, wherein the one or more diagnostic genes comprise ten or more genes selected from Table 8 or Table 9.

38. The method of claim 32, wherein the one or more reference expression profiles comprise a reference expression profile representing a disease-free human.

39. An array for use in a method for predicting a clinical outcome for an AML patient comprising a substrate having a plurality of addresses, each address comprising a distinct probe disposed thereon, wherein at least 15% of the plurality of addresses have disposed thereon probes that can specifically detect prognostic genes of AML in peripheral blood mononuclear cells.

40. The array of claim 39, wherein at least 30% of the plurality of addresses have disposed thereon probes that can specifically detect prognostic genes of AML in peripheral blood mononuclear cells.

41. The array of claim 39, wherein at least 50% of the plurality of addresses have disposed thereon probes that can specifically detect prognostic genes of AML in peripheral blood mononuclear cells.

42. The array of any one of claims 39-41, wherein the prognostic genes are selected from Tables 3, 4, 5 or 6.

43. The array of any one of claims 39-41, wherein the probe is a nucleic acid probe.

44. The array of any one of claims 39-41, wherein the probe is an antibody probe.

45. An array for use in a method for diagnosis of AML comprising a substrate having a plurality of addresses, each address comprising a distinct probe disposed thereon, wherein at least 15% of the plurality of addresses have disposed thereon probes that can specifically detect diagnostic genes of AML in peripheral blood mononuclear cells.

46. The array of claim 45, wherein at least 30% of the plurality of addresses have disposed thereon probes that can specifically detect diagnostic genes of AML in peripheral blood mononuclear cells.

47. The array of claim 45, wherein at least 50% of the plurality of addresses have disposed thereon probes that can specifically detect diagnostic genes of AML in peripheral blood mononuclear cells.

48. The array of any one of claims 45-47, wherein the diagnostic genes are selected from Table 7.

49. The array of any one of claims 45-47, wherein the probe is a nucleic acid probe.

50. The array of any one of claims 45-47, wherein the probe is an antibody probe.

51. A computer-readable medium comprising a digitally-encoded expression profile comprising a plurality of digitally-encoded expression signals, wherein each of the plurality of digitally-encoded expression signals comprises a value representing the expression of a prognostic gene of AML in a peripheral blood mononuclear cell.

52. The computer-readable medium of claim 51, wherein the prognostic gene is selected from Tables 3, 4, 5 or 6.

53. The computer-readable medium of claim 51, wherein the value represents the expression of the prognostic gene of AML in a peripheral blood mononuclear cell of a patient with a known or determinable clinical outcome.

54. The computer-readable medium of claim 51, wherein the digitally-encoded expression profile comprises at least ten digitally-encoded expression signals.

55. A computer-readable medium comprising a digitally-encoded expression profile comprising a plurality of digitally-encoded expression signals, wherein each of the plurality of digitally-encoded expression signals comprises a value representing the expression of a diagnostic gene of AML in a peripheral blood mononuclear cell.

56. The computer-readable medium of claim 55, wherein the diagnostic gene is selected from Table 7.

57. The computer-readable medium of claim 55, wherein the value represents the expression of the diagnostic gene of AML in a peripheral blood mononuclear cell of an AML-free human.

58. The computer-readable medium of claim 55, wherein the digitally-encoded expression profile comprises at least ten digitally-encoded expression signals.

59. A kit for prognosis of AML, the kit comprising: a) one or more probes that can specifically detect prognostic genes of AML in peripheral blood mononuclear cells; and b) one or more controls, each representing a reference expression level of a prognostic gene detectable by the one or more probes.

60. The kit of claim 59, wherein the prognostic genes are selected from Tables 3, 4, 5 or 6.

61. A kit for diagnosis of AML, the kit comprising: a) one or more probes that can specifically detect diagnostic genes of AML in peripheral blood mononuclear cells; and b) one or more controls, each representing a reference expression level of a prognostic gene detectable by the one or more probes.

62. The kit of claim 61, wherein the diagnostic genes are selected from Table 7.
